(19) United States
(12) Patent Application Publication
(10) Pub. No.: US 2024/0423792 A1
(43) Pub. Date: Dec. 26, 2024

(54) Devices, Systems, and Methods for a Valve Replacement

(71) Applicant: ReValve Solutions, Inc., Irvine, CA (US)

(72) Inventors: Julie Logan Sands, Irvine, CA (US); Kenneth Eugene Perry, Irvine, CA (US); Anthony Zoltan Zador, Irvine, CA (US); Samantha Opalski, Irvine, CA (US)

(73) Assignee: ReValve Solutions, Inc., Irvine, CA (US)

(21) Appl. No.: 18/826,111

(22) Filed: Sep. 5, 2024

Related U.S. Application Data
(63) Continuation of application No. 18/628,624, filed on Apr. 5, 2024.
Application No. 18/628,624 is a continuation of application No. 18/694,897, filed on Mar. 22, 2024.
Application No. 18/628,624 is a continuation of application No. 18/275,988, filed on Aug. 4, 2023.
Application No. 18/628,624 is a continuation of application No. 18/028,212, filed on Mar. 23, 2023.
Application No. 18/628,624 is a continuation of application No. 17/925,590, filed on Nov. 15, 2022.
Application No. 18/628,624 is a continuation of application No. 17/921,070, filed on Oct. 24, 2022.
(63) Continuation of application No. 17/240,914, filed on Apr. 26, 2021.
(60) Provisional application No. 63/407,624, filed on Sep. 17, 2022.
(60) Provisional application No. 63/145,878, filed on Feb. 4, 2021.
(60) Provisional application No. 63/082,035, filed on Sep. 23, 2020.
(60) Provisional application No. 63/025,881, filed on May 15, 2020.
(60) Provisional application No. 63/025,881, filed on May 15, 2020.
(60) Provisional application No. 63/015,353, filed on Apr. 24, 2020.
(60) Provisional application No. 63/025,881, filed on May 15, 2020.
(60) Provisional application No. 63/015,353, filed on Apr. 24, 2020.

(51) Int. Cl. A61F 2/24

(52) U.S. Cl. CPC A61F 2/2418; A61F 2/2439; A61F 2/2409; A61F 2210/0014; A61F 2220/0016; A61F 2220/0075; A61F 2230/0069; A61F 2250/001; A61F 2250/0039; A61F 2250/006

(57) ABSTRACT

Disclosed are valve replacement devices, systems, and methods. Valve replacement devices may comprise one- or two-piece systems comprising an adapter body and a valve assembly with leaflets positioned within the adapter body. In two-piece systems, the valve assembly may be removable from the adapter body such that both can be delivered together or separately, and the adapter body may remain implanted while the valve assembly may be removed and replaced (i.e., “revalved”). Also described are devices (such as a delivery catheter device), systems, and methods related to such delivering and revalving the valve replacement. Such delivery methods may include transseptal insertion of a new minimum leaflet structure, and securement of the valve replacement using several securement type (e.g., supra-annular, sub-annular, radial, leaflet securement, etc.). Also described is a braided helical design that mimics the heart's natural movement, and a flange structure for assisting the functioning of the valve replacement.

### CROSS-REFERENCE TO RELATED APPLICATION(S)

This application is a continuation of U.S. application Ser. No. 18/628,624, filed Apr. 5, 2024. U.S. application Ser. No. 18/628,624 is a continuation of U.S. application Ser. No. 18/694,897, filed on Mar. 22, 2024, which is a National Stage Entry of International App. No. PCT/US22/48304, filed Oct. 28, 2022, which claims priority and benefit to U.S. App. No. 63/407,624, filed Sep. 17, 2022. U.S. application Ser. No. 18/628,624 is a continuation of U.S. application Ser. No. 18/275,988, filed on Aug. 4, 2023, which is a National Stage Entry of International App No. PCT/US22/15360, filed Feb. 4, 2022, which claims priority and benefit to U.S. App. No. 63/145,878, filed Feb. 4, 2021. U.S. application Ser. No. 18/628,624 is a continuation of U.S. application Ser. No. 18/028,212, filed Mar. 23, 2023, which is a National Stage Entry of International App. No. PCT/US21/51828, filed Sep. 23, 2021, which claims priority and benefit to U.S. App. No. 63/082,035, filed Sep. 23, 2020. U.S. application Ser. No. 18/628,624 is a continuation of U.S. application Ser. No. 17/925,590, filed Nov. 15, 2022, which is a National Stage Entry of International App. No. PCT/US21/32817, filed May 17, 2021, which claims priority and benefit to U.S. App. No. 63/025,881, filed May 15, 2020. U.S. application Ser. No. 18/628,624 is a continuation of U.S. application Ser. No. 17/921,070, filed Oct. 24, 2022, which is a National Stage Entry of International App. No. PCT/US21/38886, filed Jun. 24, 2021, which claims priority and benefit to U.S. App. No. 63/015,353, filed Apr. 24, 2020, and U.S. App. No. 63/025,881. This application is a continuation of U.S. application Ser. No. 17/240,914, filed on Apr. 26, 2021, which claims priority and benefit to U.S. App. No. 63/015,353 and U.S. App No. 63/025,881. The contents of the above-referenced applications are incorporated herein by this reference as though set forth in their entirety.

### FIELD OF USE

The present disclosure relates generally to replacement heart-valve technology, and more specifically to devices, systems, and methods for delivering a valve replacement or replacing a valve replacement comprising a one-piece system and a two-piece system. Aspects of the disclosure also relate to unique features of the innovative replacement heart valve technology, including a helical braided wire design of the replacement heart valve frame and a multipoint anchoring system that utilizes a combination of supra-annular anchoring that anchors to the top of the annulus of the native heart valve, sub-annular anchoring that anchors to the bottom of the annulus of the native heart valve, and selectable and customizable radial force within the replacement heart valve that anchors within the annulus of the native heart valve.

### BACKGROUND

Heart valve intervention, such as full open-heart surgery, is often required to treat diseases of one or more of the four heart valves (which work together to keep blood properly flowing through the heart). Replacement and/or repair of a heart valve is often required when a valve is “leaky” (e.g., there is valve regurgitation) or when a valve is narrowed and does not open properly (e.g., valve stenosis). Heart valve replacement, such as mitral valve or tricuspid valve replacement, typically involves replacement of the heart's original (native) valve with a replacement mechanical and/or tissue (bioprosthetic) valve. Common problems with the replacement of valves and/or the frames carrying them include degradation of the leaflets (valve-like structure); breaking or failing frames, particularly with laser-cut nitinol frames; and undesirable changing in size of the native valve annulus. Replacement heart valves pose additional problems after they are implanted. For example, the replacement valve may move or migrate after it is placed in a desired location in the heart, or its location may not permit proper directional flow of blood through other parts of the organ, such as the outflow tract of the left ventricle.

Replacement valves are also not readily retrievable, most often because such removal can damage the surrounding heart tissue. This can be particularly problematic, for example, if the replacement valve is not properly and accurately placed into position when it is implanted in the native heart, as well as when the replacement valve starts failing, which may occur soon or years after initial implantation. An additional problem is that typical replacement valves, especially laser-cut valve frames, are relatively stiff and inflexible, resulting in a valve that does not flex with the dynamic movements of the pumping heart. Such inflexible valves do not conform to such dynamic movements, which can cause trauma to the heart surfaces, cause breaks in the frame itself, and otherwise cause or exacerbate problems during or after implantation. Thus, what is needed are treatment solutions for structural heart disease (e.g., mitral valve disease) that allow for ongoing treatment options and improving the long-term health of patients. Relatedly, there is a need for an effective Transcatheter Mitral valve replacement (TMVR) that can be simply and securely delivered while providing a platform for future intervention.

Also needed are devices, systems, and methods for a valve replacement that enables compact and secure delivery into the heart and convenient control of both the valve replacement during implantation as well as the expansion and retraction of the valve replacement when being implanted or removed/replaced, preferably entirely via a catheter. Also needed are devices, systems, and methods for ensuring proper directional flow of blood through the heart during and after a valve replacement procedure. Also needed are devices, systems, and methods for ensuring that the replacement valve is placed into the proper position when being implanted in the native heart and prior to removing the current/prior valve.

Such devices, systems, and methods should provide the functionality of a one-piece system comprising both an adapter body with engaging mechanisms that secure to the heart and a valve assembly with leaflets that is positioned within the adapter body. Such devices, systems, and methods should also provide the functionality of a two-piece system comprising an adapter body and valve assembly that are compatible with each other yet wherein the valve assembly may be removable from the adapter body such that both can be delivered together or separately and such that the adapter body may remain implanted while the valve assembly may be removed and replaced. Some such devices, systems, and methods should also relate to delivering transcatheter therapies.

### SUMMARY OF THE DISCLOSURE

The following presents a simplified overview of the example embodiments in order to provide a basic understanding of some embodiments of the present disclosure. This overview is not an extensive overview of the example embodiments. It is intended to neither identify key or critical elements of the example embodiments nor delineate the scope of the appended claims. Its sole purpose is to present some concepts of the example embodiments in a simplified form as a prelude to the more detailed description that is presented herein below. It is to be understood that both the following general description and the following detailed description are exemplary and explanatory only and are not restrictive.

The present disclosure is directed to devices, systems, and methods for a valve replacement that serves the purpose of anchoring, sealing, and controlling the position of the leaflets and sub-valvular structure. The valve replacement may be highly flexible, resilient, fatigue resistant, and securable to the native valve tissue. And it is self-adapting, meaning it adapts to—and, in addition, supports—the natural movement of the heart. In a preferred embodiment, the valve replacement comprises a collapsible adapter body that attaches to the native valve tissue and provides a scaling portion. The valve replacement comprises a frame optimized for effective sealing and fixation to the valve, wherein the design of the adapting frame is anatomically inspired and designed to maximize ventricular filling and minimize outflow tract obstruction.

In some examples, the valve replacement may be a device for assisting the functioning of a heart valve. A device embodiment may include a tubular frame with an inflow end and an outflow end. In some examples, the tubular frame may include at least one braided wire wound in a helical spiral direction. The helical spiral direction may begin at the inflow end and end at the outflow end. The tubular frame may be configured to lengthen and compress in relation to a heart contraction.

The valve replacement—whether as a one- or two-piece system—may further comprise a valve assembly, wherein the valve assembly comprises leaflets and is compatible to reside within the adapting frame. In some examples, a valve assembly may have a tubular braided frame and have an inflow end and an outflow end, as well as at least one commissure post at the outflow end. In some examples, the valve assembly may also include a leaflet assembly connected to the at least one commissure post. The leaflet assembly may also be configured to provide a seal between the inflow end and the outflow end.

The present disclosure also provides for a one- or two-piece valve replacement system that—due to its braided-wire frame design—is compressible to a smaller profile when compared to the prior art, wherein the smaller compressed profile allows for delivery via not only transapical approaches but also transfemoral and transseptal approaches. In embodiments, the valve replacement is constructed using a braided wire that is wrapped in an over-under fashion permitting the apices and crossing points of the braided wire structure to have a cylindrical helical movement, wherein the structure is free to move within a helical spiral form. In embodiments, shape set fabric and sewn nodes using sutures to sew the fabric to the frame provide upper and lower constraints within which the braided wire frame structure is still able to move with the helical movement of the heart. In embodiments, anchor features of the valve replacement can be braided with a cylindrical longitudinal flexing symmetry on a helical axis. In other embodiments, the anchor features or commissure posts of the valve replacement can be welded onto the braided frame separately using different types of welding techniques, such as hypotubes or wire to wire welding. In other embodiment, the anchors can be welded onto the replacement valve by replacing sections of the braided wire frame. Embodiments can also utilize techniques to optimize the valve replacement by, for example, optimizing the profile of the valve replacement by electropolishing the anchor features or braided wire frame structure.

The two-piece system disclosed herein allows for a further lower profile because the adapting frame and the valve assembly may be delivered as two separate devices. In one embodiment, a device for assisting the functioning of a heart valve may include a tubular braided frame with an inflow end and an outflow end, and a flange structure at the inflow end of the tubular braided frame. The device embodiment may also have at least one feature at the outflow end of the tubular braided frame configured to anchor to a native leaflet, and at least one feature at the outflow end of the tubular braided frame configured to anchor to an area adjacent to the native annulus. The device embodiment may also have at least one commissure post at the outflow end of the tubular braided frame (and the commissure post may extend out from the tubular braided frame). The device embodiment may also have a leaflet assembly connected to the at least one commissure post. In some examples, the connection between the leaflet assembly and the commissure post may extend out from the tubular braided frame. In some examples, the leaflet assembly may be configured to provide a seal between the inflow end and the outflow end of the tubular braided frame.

In another embodiment, a device for assisting the functioning of a heart valve may include an adapter having a tubular braided adapter frame with an inflow end and an outflow end, a flange structure at the inflow end of the tubular braided adapter frame, and at least one anchor at the outflow end of the tubular braided adapter frame. In some examples, the device embodiment may further include a valve assembly. In some examples, the valve assembly may have a tubular assembly frame comprising a second inflow end and a second outflow end.

In some examples, the valve assembly may further include a leaflet assembly. In some examples, the leaflet assembly may be configured to provide a seal between the second inflow end and the second outflow end. In some examples, the tubular assembly frame of the device embodiment may be braided. In some examples, the valve assembly may include at least one commissure post at the second outflow end, and the leaflet assembly may be connected to the at least one commissure post. The valve assembly of the device embodiment may be configured to removably engage with the adapter. In some examples, the inflow end of the adapter may be proximal in location to the second inflow end and the outflow end of the adapter may be proximal in location to the second outflow end.

Relatedly, devices, systems, and methods for delivering a valve replacement are also described herein. One method embodiment of delivering a heart valve may include the step of advancing a catheter device for carrying a heart valve toward a mitral annulus. The method embodiment may also include the step of pushing the catheter device through the mitral annulus.

The method embodiment may also include the step of deploying at least one engagement attachment from the catheter device embodiment in a ventricle. The method embodiment may also include the step of securing, in the ventricle, the at least one engagement attachment clip to at least one native leaflet. The method embodiment may also include the step of deploying at least one anchor from the catheter device in the ventricle. The method embodiment may also include the step of securing, in the ventricle, the at least one anchor to native heart tissue. The method embodiment may also include the step of releasing, in the atrium, a flange to fit over the mitral annulus. In some examples, the at least one engagement attachment may include or be at least one clip.

An embodiment of a delivery catheter device may include a steerable distal end comprising a nose cone, which may be configured to approach a mitral valve upon transeptal entry into the atrium. The device embodiment may also include a proximal end separated a length from and connected to the distal end. The device embodiment may also include a deployable adapter and a sheath spanning at least some of the length between the distal end and the proximal end. The device embodiment may also include at least one protactable anchor over at least part of the adapter. The device embodiment may also include a protractable flange located toward the proximal end from the adapter.

The present disclosure also provides for a “re-valvable” system, method, and device, where the leaflet structure of the replacement heart valve can be removed and replaced by another leaflet structure. One method embodiment may include the step of replacing a heart valve may include the step of in the atrium, transeptally advancing a first catheter device towards the mitral annulus. In some examples, the first catheter device may have a separable new minimum leaflet structure (MLS).

The method embodiment may also include the step of positioning the first catheter device so that the new MLS is aligned with and proximate to the mitral annulus. In some examples, the method embodiment may also include the step of, in the ventricle, transapically pushing a second catheter device towards the mitral annulus. In some examples, the second catheter device may be configured to remove an old MLS. In some examples, the method embodiment may also include the step of positioning at least part of the second catheter device to transapically grasp the old MLS from the mitral annulus. In some examples, the method embodiment may also include the step of securing and pulling, using at least part of the second catheter device, the old MLS away from the mitral annulus for transapical removal.

In some examples, the method embodiment may also include the step of transeptally inserting, using the first catheter device, the new MLS into the mitral annulus. In some examples of the method embodiment, the transeptally inserting further comprises inserting the new MLS into the adapter. Also, in some examples of the method embodiment, the old MLS may be within an adapter inside the mitral annulus, and the second catheter device may be configured to remove an old MLS from within the adapter.

Also described herein is a device for assisting the functioning of a heart valve. The device may include a flange structure for placement at an inflow end of a heart valve adapter frame. In some embodiments, the flange structure may include a top plate having a D-shaped perimeter ledge having a first underside surface configured for placement over at least some native tissue. The flange structure may also include, interior to and topographically below the top plate, a first contoured ring having a second underside surface configured for placement over at least some of the native tissue. The flange structure may further include interior to and topographically below the first contoured ring, a second contoured ring having a third underside surface configured for placement over at least some of the native tissue.

Still other advantages, embodiments, and features of the subject disclosure will become readily apparent to those of ordinary skill in the art from the following description wherein there is shown and described a preferred embodiment of the present disclosure, simply by way of illustration of one of the best modes best suited to carry out the subject disclosure. As will be realized, the present disclosure is capable of other different embodiments and its several details are capable of modifications in various obvious embodiments all without departing from, or limiting, the scope herein. Accordingly, the drawings and descriptions will be regarded as illustrative in nature and not as restrictive.

### BRIEF DESCRIPTION OF THE DRAWINGS

The accompanying drawings, which are incorporated in and constitute a part of this specification, illustrate embodiments of the disclosure and together with the general description of the disclosure given above and the detailed description of the drawings given below, serve to explain the principles of the disclosure. In certain instances, details that are not necessary for an understanding of the disclosure or that render other details difficult to perceive may have been omitted.

FIG. 1 generally illustrates an embodiment of a valve replacement as disclosed herein.

FIGS. 2A-2C generally illustrate an embodiment of a valve replacement as disclosed herein.

FIGS. 2D-2E generally illustrate an embodiment of a valve replacement as disclosed herein.

FIG. 3 generally illustrates an embodiment of a valve replacement as disclosed herein.

FIG. 4 generally illustrates an embodiment of a valve replacement as disclosed herein.

FIG. 5 generally illustrates an embodiment of a valve replacement as disclosed herein.

FIGS. 6A and 6B generally illustrate embodiments of a valve replacement as disclosed herein.

FIGS. 7A-7D generally illustrate embodiments of a valve replacement as disclosed herein.

FIG. 8A generally illustrates the helical functionality of the human heart.

FIG. 8B generally illustrates an embodiment of a valve replacement as disclosed herein.

FIGS. 8C-F generally illustrate embodiments of a valve replacement as disclosed herein.

FIG. 8G generally illustrates areas of a heart as disclosed herein.

FIGS. 9A and 9B generally illustrate an embodiment of a valve replacement as disclosed herein.

FIGS. 9C and 9D generally illustrate an embodiment of a valve replacement as disclosed herein.

FIGS. 10A and 10B generally illustrate embodiments of a valve replacement as disclosed herein.

FIGS. 11A and 11B generally illustrate embodiments of a valve replacement as disclosed herein.

FIGS. 12A-12E generally illustrate embodiments of a valve replacement as disclosed herein.

FIGS. 13A-13D generally illustrate embodiments of a valve replacement as disclosed herein.

FIGS. 13E-13H generally illustrate embodiments of a valve replacement as disclosed herein.

FIGS. 14A-14E generally illustrate embodiments of a valve replacement as disclosed herein.

FIGS. 15A and 15B generally illustrate embodiments of a valve replacement as disclosed herein.

FIGS. 16A-16D generally illustrate embodiments of a valve replacement as disclosed herein.

FIGS. 17A-17D generally illustrate embodiments of a valve replacement as disclosed herein.

FIGS. 18A and 18B generally illustrate embodiments of a valve replacement as disclosed herein.

FIGS. 19A and 19B generally illustrate embodiments of a valve replacement as disclosed herein.

FIG. 20 generally illustrates an embodiment of a valve replacement as disclosed herein.

FIGS. 21A-I generally illustrate an embodiment of a valve replacement as disclosed herein.

FIGS. 21J-M generally illustrate an embodiment of a valve replacement as disclosed herein.

FIGS. 21N-R generally illustrate an embodiment of a valve replacement as disclosed herein.

FIGS. 22A and 22B generally illustrate an embodiment of a valve replacement as disclosed herein.

FIGS. 23A and 23B generally illustrate an embodiment of a valve replacement as disclosed herein.

FIG. 24A generally illustrates an embodiment of a valve replacement as disclosed herein.

FIG. 24B generally illustrates an embodiment of a valve replacement as disclosed herein.

FIGS. 25A and 25B generally illustrate an embodiment of a valve replacement as disclosed herein.

FIGS. 25C-25E generally illustrate embodiments of the valve replacements as disclosed herein.

FIGS. 25F-25H generally illustrate an embodiment of a wire frame for valve replacements as disclosed herein.

FIG. 26 generally illustrates an embodiment of a valve replacement as disclosed herein.

FIG. 27 generally illustrates an embodiment of a valve replacement as disclosed herein.

FIGS. 28A-F generally illustrate an embodiment of a valve replacement as disclosed herein.

FIGS. 29A and 29B generally illustrate an embodiment of a valve replacement as disclosed herein.

FIGS. 30A-D generally illustrate an embodiment of a valve replacement as disclosed herein.

FIGS. 31A-F generally illustrate an embodiment of a valve replacement as disclosed herein.

FIGS. 32A-F generally illustrate an embodiment of a valve replacement as disclosed herein.

FIGS. 33A-F generally illustrate an embodiment of a valve replacement as disclosed herein.

FIGS. 34A-C generally illustrate an embodiment of a valve replacement as disclosed herein.

FIG. 35 generally illustrates an embodiment of a delivery system as disclosed herein.

FIG. 36 generally illustrates an embodiment of a delivery system as disclosed herein.

FIG. 37 generally illustrates an embodiment of a delivery system as disclosed herein.

FIG. 38 generally illustrates an embodiment of a delivery system as disclosed herein.

FIG. 39 generally illustrates an embodiment of a delivery system as disclosed herein.

FIG. 40 generally illustrates an embodiment of a delivery system as disclosed herein.

FIG. 41A generally illustrates a step for an assembly embodiment as disclosed herein.

FIG. 41B generally illustrates a step for an assembly embodiment as disclosed herein.

FIG. 42 generally illustrates a step for an assembly embodiment as disclosed herein.

FIG. 43 generally illustrates a step for an assembly embodiment as disclosed herein.

FIG. 44 generally illustrates a step for an assembly embodiment as disclosed herein.

FIG. 45 generally illustrates a step for an assembly embodiment as disclosed herein.

FIG. 46 generally illustrates a step for an assembly embodiment as disclosed herein.

FIG. 47 generally illustrates a step for an assembly embodiment as disclosed herein.

FIG. 48 generally illustrates a step for an assembly embodiment as disclosed herein.

FIG. 49 generally illustrates a step for an assembly embodiment as disclosed herein.

FIG. 50 is a flow diagram generally illustrating a method of delivering a heart valve as disclosed herein.

FIG. 51 is a flow diagram generally illustrating a method of replacing a heart valve as disclosed herein.

### DETAILED DESCRIPTION OF EMBODIMENTS

Before the present systems and methods are disclosed and described, it is to be understood that the systems and methods are not limited to specific methods, specific components, or to particular implementations. It is also to be understood that the terminology used herein is for the purpose of describing particular embodiments only and is not intended to be limiting. Various embodiments are described with reference to the drawings. In the following description, for purposes of explanation, numerous specific details are set forth in order to provide a thorough understanding of one or more embodiments. It may be evident, however, that the various embodiments may be practiced without these specific details. In other instances, well-known structures and devices are shown in block diagram form to facilitate describing these embodiments.

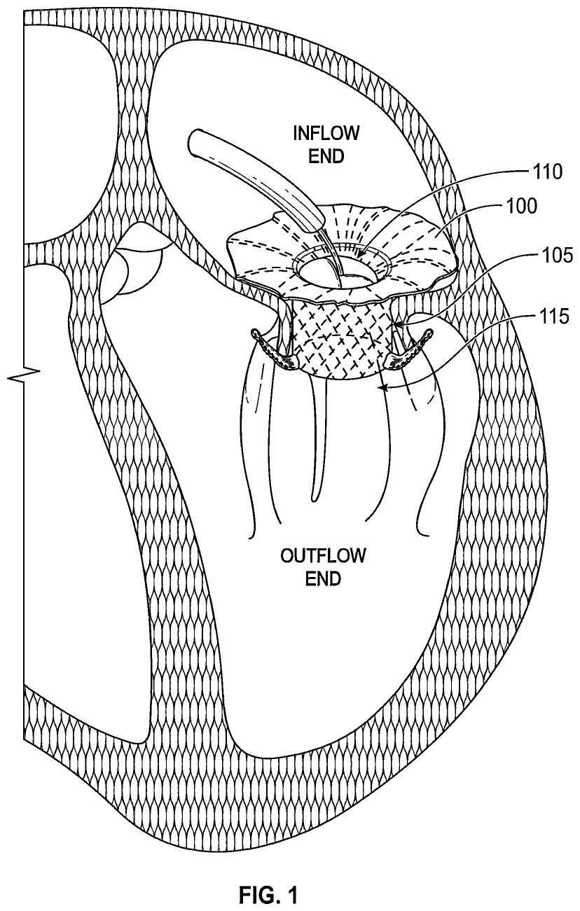

FIG. 1 generally illustrates an embodiment of a valve replacement as disclosed herein. FIG. 1 discloses an embodiment of a valve replacement (“valve replacement”) 100 implanted in a malfunctioning mitral valve 105. The valve replacement 100, however, is not limited to compatibility with only the mitral valve 105 and may be also implanted in the tricuspid, aortic, or pulmonary valves (not shown). In a preferred embodiment, the valve replacement 100 comprises a braided, collapsible frame and a braided valve-and-leaflet assembly that together serve to provide a scaling portion. The valve replacement may have an inflow end 110 (shown as facing the top side of the mitral valve 105) and an outflow end 115 (shown as facing the bottom side of the mitral valve 105).

As set forth herein, the compatibility of the collapsible frame and leaflet assembly may be performed in various embodiments. In one embodiment, the valve replacement 100 may comprise the frame and valve assembly as a two-piece apparatus (referred to herein for case of reference as the “two-piece system”). In another embodiment, the valve replacement 100 may comprise the frame and valve assembly as a one-piece apparatus (referred to herein for case of reference as the “one-piece system”). Regardless of the embodiments, the valve replacement 100 may further comprise attachments and additional features for catheter delivery, positioning, partial deployment, and retrieval.

### Two-Piece Valve Replacement Overview

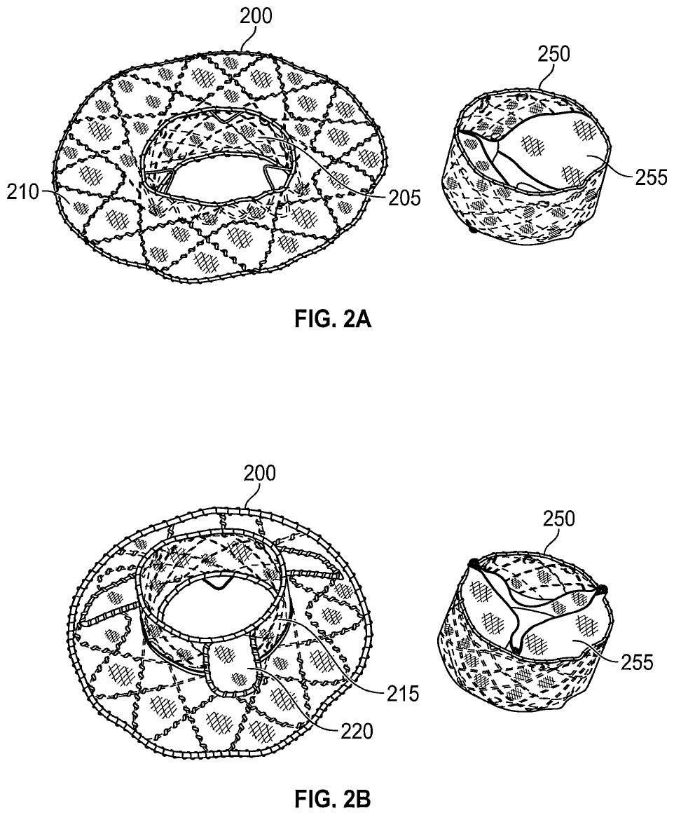

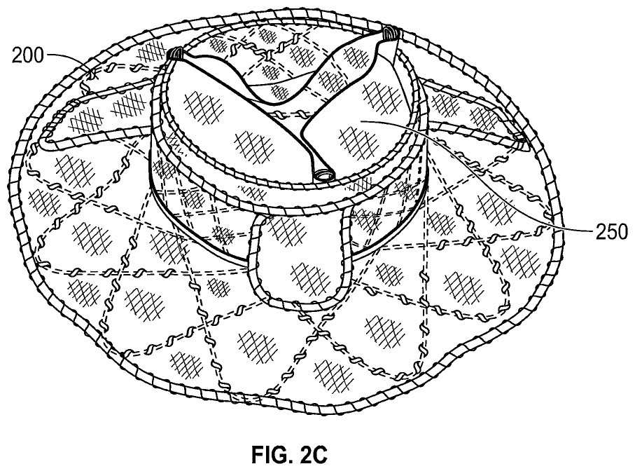

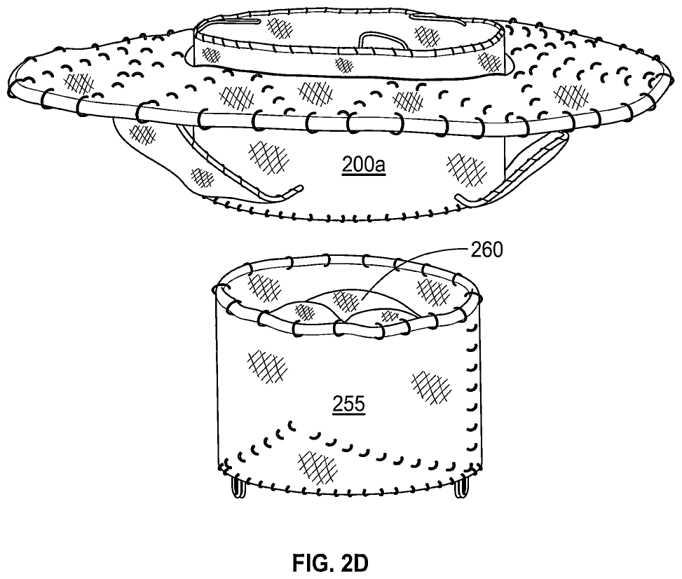

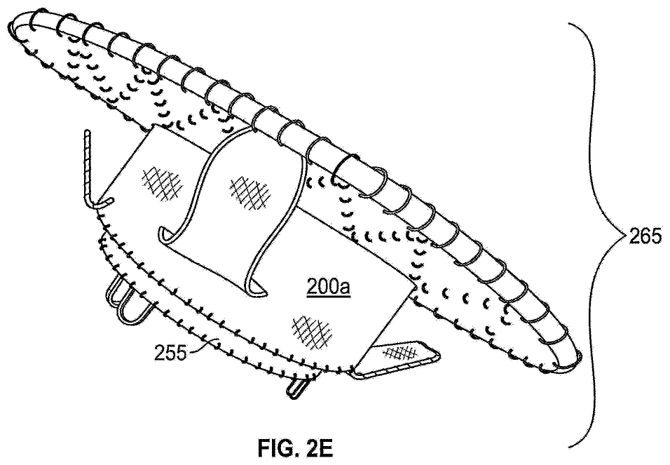

FIGS. 2A-2C generally illustrate an embodiment of the valve replacement 100a as disclosed herein. FIGS. 2A-2C disclose embodiments of the two-piece system 100a. As shown in FIG. 2A, the two-piece system 100a comprises a heart valve frame 200 (referred to herein for ease of reference as the “adapter”) and a heart valve assembly 250 (referred to herein for case of reference as the “valve assembly”). In one embodiment, the adapter 200 comprises an opening 205 that is compatible with the valve assembly 250. The adapter 200 further comprises a sealing skirt 210 at the top, a body portion 215, and one or more anchors 220 extending out from the bottom of the body portion 215. In an embodiment, the valve assembly 250 comprises a leaflet-structure component 255 that enables blood flow through the valve assembly 250.

FIG. 2C discloses the valve replacement as a two-piece system 100a wherein the adapter 200 and the valve assembly 250 are cooperatively sized and configured together. The adapter 200 and the valve assembly 250 may fit as a single unit and be compressed to be inserted into a heart catheter for delivery to a target valve, i.e., in an as-connected form where the two portions are mechanically linked together. This configuration advantageously allows the delivery and control of both portions of the valve replacement 100a.

The adapter 200 and the valve assembly 250 may also be carried in a delivery catheter in an unconnected form where the two portions are not mechanically linked together. This configuration advantageously allows the delivery catheter to independently control each of the portions and can also increase the flexibility and torsion characteristics of the delivery catheter containing the two portions, which can be advantageous both while conveying the delivery catheter to through the patient's body, the vasculature, the desired target, and while delivering the replacement valve at/to the target. In such embodiments, the adapter 200 and the valve assembly 250, as separately delivered portions, may both be further compressed, enabling a low profile that is conducive to delivery via blood vessels that may not be sufficiently healthy or wide in size so as to allow delivery of both portions as a single unit.

FIGS. 2D-2E generally illustrate an embodiment of the valve replacement as disclosed herein. FIG. 2D shows another embodiment of the valve replacement as a two-piece system that includes two independent devices deliverable as one system, such as the adapter 200a and the MLS″) 255. The adapter 200a may be configured to conform and fix to a native valve (as explained in more detail below). In some embodiments, the adapter 200a may be designed to be conformable to the human anatomy and provide fixation to the native valve and interact with native heart tissue. In some embodiments, the MLS may house the leaflets of the replacement heart valve. In some examples, the leaflets 260 of the MLS 255 may be made from materials that include bovine pericardium. The MLS 255, in some embodiments, may be configured for secure placement within the adapter 200a. In some embodiments, the adapter 200a and the MLS 255 are cooperatively sized and may be configured together for proper placement, and delivered as one system to the patient through a delivery systems and methods described herein.

FIG. 2E shows how the adapter 200a and the MLS 255 may fit as a single unit 265 and be compressed. Such compression may facilitate insertion into a heart catheter for delivery to a target valve, i.e., in an as-connected form where the two portions are mechanically linked together.

Once installed and delivered (as explained in more detail below), the two separate devices of the adapter 200a and the MLS 255 may also work together as a single unit 265 or system.

As with the adapter 200a and the valve assembly 250 shown in FIG. 2D, the adapter 200a and the MLS 255 of FIG. 2E, may also be carried in a delivery catheter in an unconnected form where the two portions are not mechanically linked together, and both portions may also be delivered as a single unit 265.

### One-Piece Valve Replacement Overview

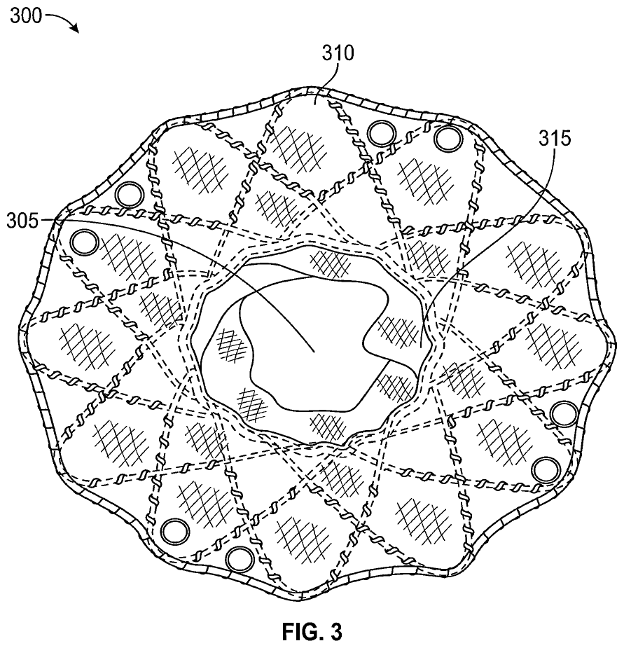

FIG. 3 generally illustrates an embodiment of the valve replacement as disclosed herein. FIG. 3 discloses an embodiment of the one-piece system 300, comprising an opening 305 for blood flow, a sealing skirt 310, and a leaflet structure 315. Though not shown in FIG. 3, the one-piece system 300 further comprises a body below the sealing skirt 310 that is similar to the body of the adapter 200 in FIGS. 2A-2C and may further comprise anchors similar to anchors of the adapter 200 in FIGS. 2A-2C. In one embodiment, the one-piece system 300 may function as a permanent implant.

Whether as a one- or two-piece system, the valve replacement allows for valve-in-valve placement, wherein embodiments of the valve-in-valve placement comprise replacing existing leaflets and valve assemblies without a reduction in area (such as by placing new material over existing material), and without compromising the functionality of the implanted valve replacement.

### Braided Structures

The braided structures disclosed herein are applicable to the one-piece system and to the adapter and the valve assembly of the two-piece system. Thus, though various embodiments of braided structures may be shown in relation to the adapter and the valve assembly, it should be understood that such embodiments are also in relation to the one-piece system.

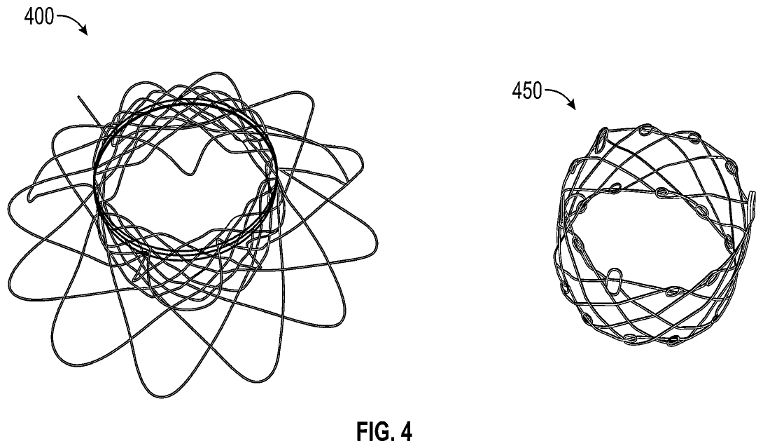

FIG. 4 generally illustrates an embodiment of a valve replacement as disclosed herein. FIG. 4 discloses the braided wire frame of an adapter 400 and the braided wire frame of a valve assembly 450. The braided wire frame allows the adapter 400 and the valve assembly 450 to be compressed, which when released may expand in size. Similarly, the one-piece system may also be compressed and expanded. The braided wire frame design thus enables the valve replacement to be compressed to a small diameter—such as 4 mm to 6 mm—such that it may be delivered in a catheter. The braiding of the wire and overlapping with other wires also reduces or eliminates fracturing of the wire because of the decreased stress on the frame. The braiding also enables various-sized wires to be used.

The braided wire frame of the one-piece system, the adapter 400, and valve assembly 450 may comprise various wire embodiments, such as a single wire, two or more wires (for example, grafted or welded together), and a wire spliced of multiple wires. The wire(s) making up the one-piece system and the two-piece system may be constructed of varying material, such as nitinol, which has shape-memory characteristics and varies in dimensions, such as in diameter size.

By integrating diverse wire thicknesses and braiding designs, the valve replacement conforms with various densities and characteristics (i.e., radial force and expansion) of the heart's anatomy. In this, the braided frame enables the valve replacement to have a flexible and conformable performance, wherein the valve replacement self-adapts and moves with the heart while being forgiving to anatomical anomalies—similar to the heart's helical structure, as will be disclosed herein. The braided frame also facilitates placement of the valve replacement, maximizes its seal, and prevents migration with an integrated and optimized anchoring system. The braided frame geometry of the valve replacement allows for diverse application, such as being customizable to mitral and tricuspid anatomies; allows for fewer sizes to be needed to treat most disease states; promotes rapid prototyping; allows incorporation of various design features; promotes quicker design advancement with rapid evaluation and optimization of features; and is scalable using conventional processes. The braiding structure also allows for more degrees of freedom and opportunities for the wires to be in various positions.

An embodiment of fabricating the braided wire frame comprises oversizing the braided wire frame in relation to heart valve, which allows for more radial force for the same amount of material and geometry, thus allowing the frame to open up more fully and function better. Furthermore, it decreases the manufacturing tolerances involved in manufacturing the valve replacement. Oversizing the braided frame biases the wire frame structure so that there is less motion between the wires as they are predisposed with elastic strain energy to conform and adapt with greater radial force. As a result, the valves have higher degrees of consistency and the manufacturing tolerances associated with attaching the leaflets, for example, are greatly improved.

In one embodiment, the braided frame is wrapped and shape-set such that it has enough radial force to self-expand and be opened up to desired radial capacity while still being configured to fit within a catheter.

Embodiments of the valve replacement may range in diameter from 25.0 mm to more than 55 mm. In another embodiment, the wire frame is oversized, which comprises braiding the wire frame on a mandrel that is 25.4 mm in diameter (or 28.0 mm or 32.0 mm, depending on the desired valve size) and shape-setting it by treating it in 505° C. salt/sand bath. The frame is then removed from the initial mandrel and stretched over a 29.0 mm mandrel (or 31.0 mm or 33.0 mm, e.g., for larger valves) and shape-set again. Temporary strings (or other similar methods known to one skilled in the art) are then run through the loops and tied using a 25.4 mm mandrel as a reference diameter for the valve frame. This compresses the frame by spring loading the loops (though other embodiments may comprise other structures beyond loops, such as simple apices). The braided valve replacement may thus be shape-set at a larger diameter and then constrained to a smaller diameter and held with string until fabric is sewn onto the frame. In another manufacturing embodiment, the wire frame repeats a braid pattern over its length three times while wrapping five times around a circle.

The braided wire architecture of embodiments of the valve replacement provides significant advantages over valve architectures that rely on laser cut frames or that have cell structures with fixed nodes along the replacement valve frame instead of a helical over-under braid pattern that permits the replacement valve frame to move with the natural helical movement of the native heart. Braided structures, such as those described herein in certain embodiments, provide collapsible scaffolding with a greater range and ability to contour to the native heart structure because the “nodes,” where wires are wrapped in an over-under braiding style, may in some examples not be fixed and may be slid across each other to accommodate anatomical contouring. Such unfixed, sliding nodes having an over-under braiding style may allow greater flexibility and mobility than a pattern of fixed immovable nodes at intersection points of wires. Relatedly, in manufacturing, the flange embodiments deliberately position the most outer ring of braided nodes outward to minimize leakage between the braided wires and enhance the stiffness of the “D” perimeter.

Embodiments of the valve replacement may comprise compatibility with various-size catheters, such as 26F, 28F, 30F, 32F, and 34F.

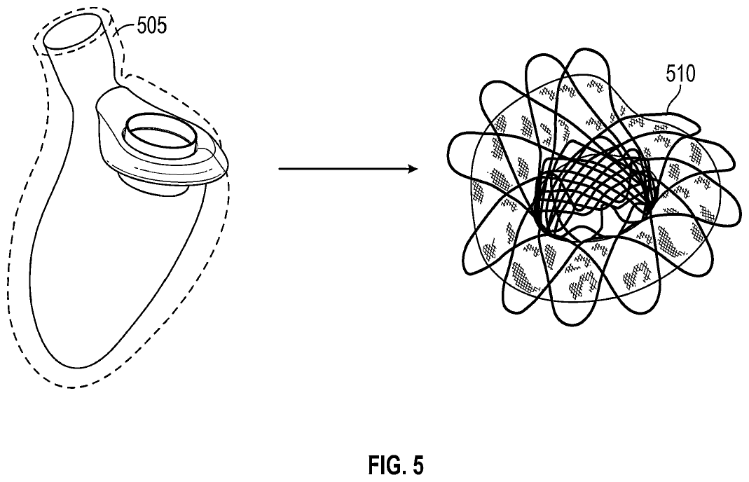

FIG. 5 generally illustrates an embodiment of a valve replacement as disclosed herein. As shown in FIG. 5, an adapter 510 may be compatible with a human heart 505, wherein embodiments of achieving coaptation comprise sealing and anchoring by adjustment of the over- and -under pattern of the braid to realize separable sections of the braid that can behave independently. The adapter 510 may be constructed of varying material and vary in dimensions. In one embodiment, the adapter 510 may be made up of a nitinol wire braid of one or more wires with different diameters. When released the adapter may expand in size (e.g., the body expanding to 25 mm or greater in diameter and the sealing skirt expanding anywhere from 40 mm to 70 mm in diameter).

The valve replacement may comprise other types of wire, such as stainless steel, cobalt chrome, and other types of implant metals. In other embodiments, the valve replacement may comprise polymer materials, such as biocompatible plastics and fiber-reinforced polymer. Some embodiments may comprise drawn-filled tubing (outside material NiTi and inside material some higher radiopaque material) for the valve replacement or portions of the valve replacement (e.g., anchors, or features desired to be seen under fluoroscopy). The valve replacement or portions of it may be made of hollow tubing. Additionally, flat wire or other cross-sections of wire may be chosen for portions of the valve replacement, such as to provide tailored/increased stiffness for anchors.

### Flanges and Anchors of the Braided Wire Frame

The adapter is designed to preserve native ventricular filling by orienting flow into the ventricle in such a way as to limit turbulence and maximize efficient flow, such as towards the ventricle wall, between the papillary muscles, or otherwise oriented towards the apex of the ventricle (“virtual apex”).

The adapter is also designed to be anatomically customized with patient and disease state-specific sizing. Sizing may be based on anatomical data; for example, using a sizing tool to determine adapter diameter and flange length, while also optimizing valve orientation for both ventricle outflow consideration and ventricular efficiency. In the example, parameters of the sizing tool are fed to the parametric device model, which automatically creates the pattern for the shape-set tooling.

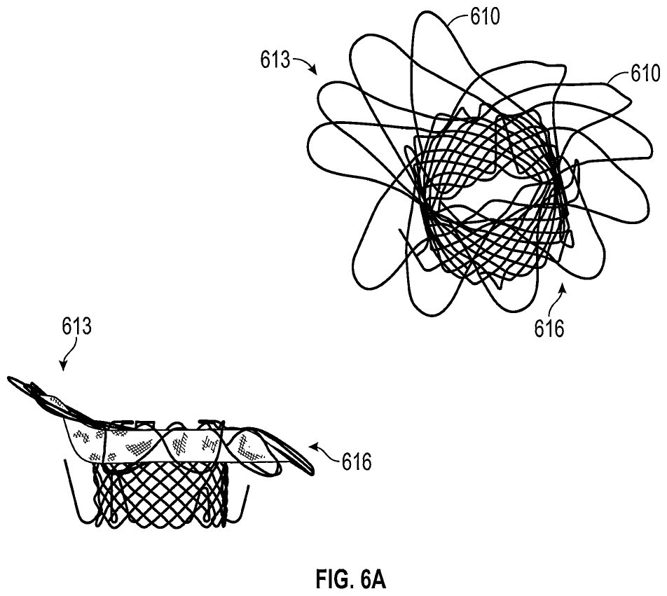

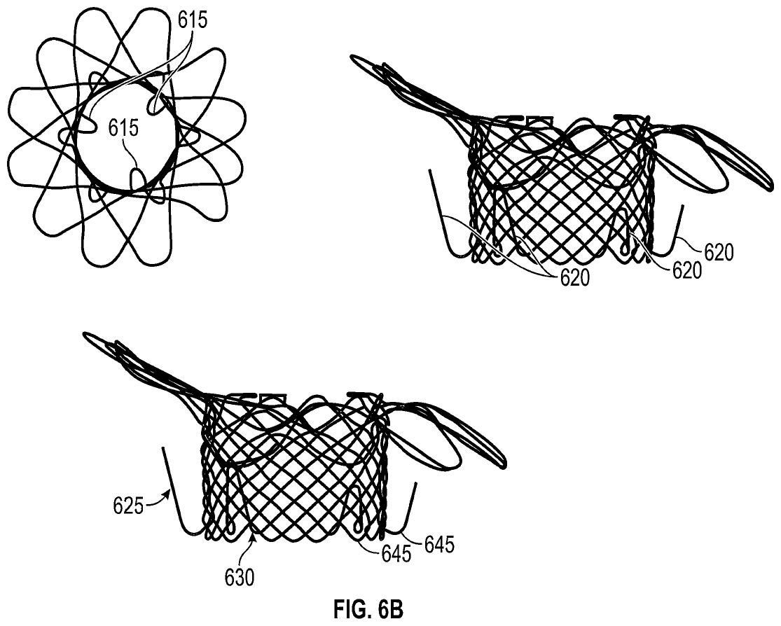

FIG. 6A generally illustrates an embodiment of the valve replacement as disclosed herein. As shown in FIG. 6A, the adapter may comprise an adapter body 605 and one or more atrial flanges 610. In one embodiment wherein the adapter is applied to a valve, such as a mitral valve, the circumference of the atrial flange 610 is separated into one-third 613 and two-thirds 616. The one-third portion 613 of the atrial flange 620 engages with the fibrous aorta-mitral curtain and is formed at an angle that prevents the valve being pulled into the LVOT. This feature also maximizes sealing during systole. The two-thirds portion 616 of the atrial flange 620 engages the muscular wall and is formed at an angle that pulls the valve away from the LVOT and directs flow towards the apex of the ventricle, between the papillary muscles, or towards the ventricle wall. In other embodiments, the adapter body and atrial flange function similarly or identically when applied to the tricuspid valve.

FIG. 6B generally illustrates an embodiment of the valve replacement as disclosed herein. As shown in FIG. 6B, the adapter may comprise valve and retainers 615 within the inner frame of the adapter body. The adapter may also comprise sub-valvular anchors 620 for leaflet management. In one embodiment, the sub-valvular anchors 620 are made up of one or more of the following: anterior leaflet anchor 625, anchor strut 630, and posterior leaflet anchors 645. For example, the adapter may comprise a single anterior leaflet anchor clip 625, two anchor struts 630, and three posterior leaflet anchor clips 645. The anchors may be configured to be biased in an upward direction so as to be radially overlapping in relation to the adapter body. In embodiments, the anchor struts are configured to land, or anchor next to, fibrous landing zones within the native heart tissue and near the native heart valve being replaced. For example, in embodiments, the anchor struts are configured to be positioned posterior to the trigones in fibrous landing zones of the native heart. In embodiments, the anchor struts are also configured to be positioned posterior to fibrous or muscular landing zones near the anterior or posterior leaflets of the native heart.

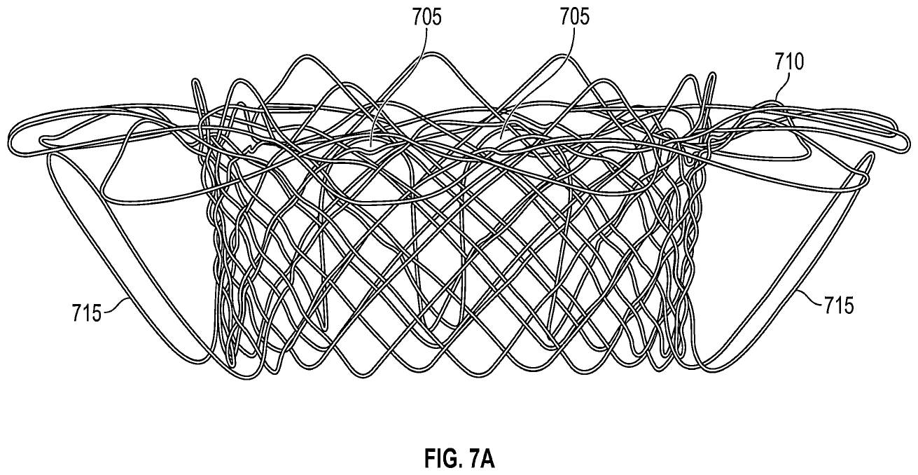

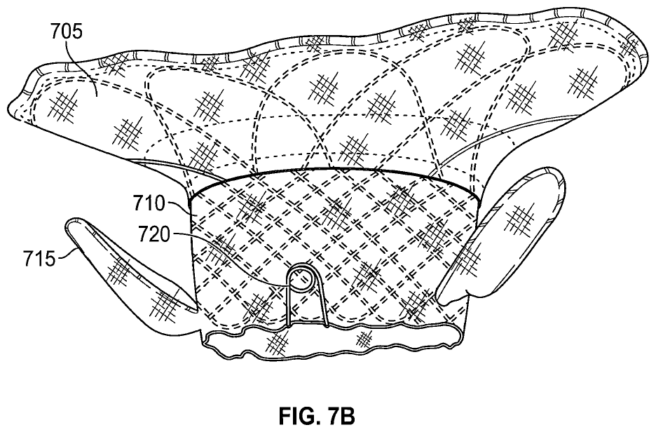

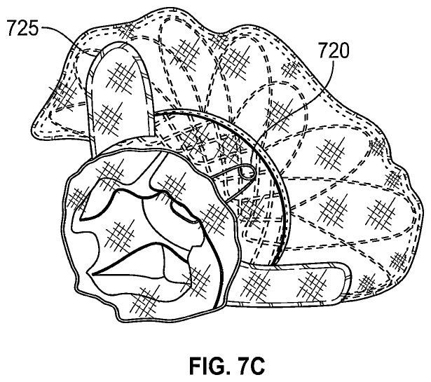

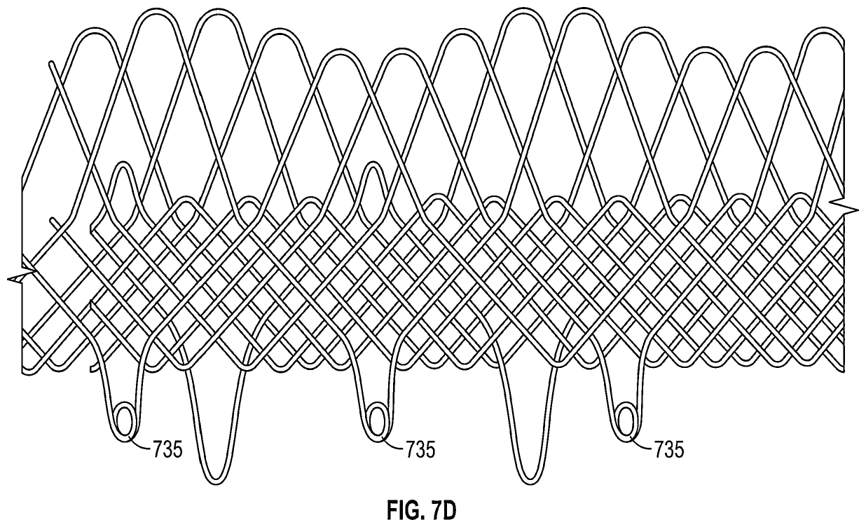

FIG. 7A generally illustrates an embodiment of the valve replacement as disclosed herein. FIG. 7A shows an embodiment of a wire braid frame that the adapter is comprised of. The wire braid frame may comprise a 24-point braid pattern, with double posterior leaflet anchors 705, wherein the double posterior leaflet anchors 705 are used to maintain symmetry and additionally provide twice the structural anchoring. The wire braid frame may also comprise dual stabilization anchors 710. Also shown is that the wire braid frame may have the anchor locations available in 15-degree increments.

The anchors may be, in some embodiments, an extension of the tubular braided frame and extend out from the outflow end to function as an engagement attachment. In other embodiments, the wire braid frame of an adapter may have anchors that are grafted, welded, or fused on. For example, FIG. 7A shows the combination of a larger gage wire (0.0175″-0.02″) (represented by the stabilization anchors 710) and smaller gage wire (0.012-0.0175″) (represented by the posterior leaflet anchors 705 and further represented by additional wires 715) by means of a joining operation at the interface between the varying-size wires. The connection interface may be a weld or a weld with a support tube.

Embodiments of welding used may be in relation to the material that the valve replacement is comprised of. In an embodiment of the anchors comprising a hollow tubing (hypotube) material, the inside diameter of the hypotube mates perfectly with the diameter of the wire so that a simple weld or other helical weld pattern may be used to join the anchor to the frame. There, the ends of the hypotube may be chamfered so as to present a smooth transition with the attached wire. Radiopaque wire may be inserted inside the hypotube and positioned to be at the peaks of the anchors (such embodiment provides optimal fluoroscopic visualization) or anywhere along the hypotube for clinical visualization. In embodiments, hypotube anchors are also preferable because the hypotube material can be chosen to have a greater stiffness or strength than the other wires used for the helical braided architecture of the replacement valve. Moreover, the hypotube material can be shaped to provide a longer surface area along a distal tip of the hypotube anchor that presses against the native heart anatomy to prevent migration of the replacement heart valve. A combination of greater stiffness and a longer surface area along a distal tip of the hypotube anchor distributes the anchor force of the replacement heart valve along a wider or greater area of the native heart anatomy, thereby decreasing the chances of damage to the native heart anatomy. Moreover, hypotube anchors provide opportunities for greater customization of the anchoring system because the hypotube anchor material can be sized and selected based on desired stiffness and contact area at the distal end of the anchor that anchors to the native heart anatomy.

FIGS. 7B and 7C generally illustrate embodiments of the valve replacement as disclosed herein. The valve replacement embodiments of FIGS. 7B and 7C may include one or more anchor features. In one embodiment, as shown in FIG. 7B, the valve replacement may comprise an atrial sealing skirt 705, a frame body 710, and an anchor feature 715 that are covered in a fabric for the purpose of flow sealing and/or encouraging (e.g., influencing either promoting or inhibiting) tissue growth after implantation. The anchor feature 715 may be strut or a stabilization anchor 715, which may assist in preventing migration of the valve replacement into the atrium. In some examples, the stabilization anchor 715 or a portion thereof may be configured or designed to rest on a specific area of the annulus (e.g., the trigone area). The embodiment may further comprise an anchor feature 720 that in some embodiments may not be covered in a fabric. In some embodiments, that anchor feature 720 may be or include a clip 720 or a hanger designed or configured to hold native leaflets. FIG. 7C shows a valve replacement comprising anchor features that may be posterior leaflet anchors (or struts) 725 and clips 720. In other embodiments, the clips and anchors are covered in fabric.

FIG. 7D generally illustrates an embodiment of the valve replacement as disclosed herein. FIG. 7D shows an embodiment of the valve replacement comprising a clip component 735 for the purpose of improving delivery control, via secure attachment of the valve replacement to a delivery catheter, and for the purpose of improving the efficiency and efficacy of leaflet attachment. FIG. 7D shows a flat-pattern schematic of a wire frame with a clip 735, wherein the clip 735 may be a looped portion of the wire frame extending out from the main body of the wire frame. In some embodiments, a clip 735 may be positioned at two or more separate locations around the circumference of the valve replacement. In other embodiments, clips 735 may be shape-set 180° such that they can provide for a hook shape to clip onto the native valve leaflets. For example, once the valve replacement is released from a delivery system, the clips 735 may attach onto the native valve leaflets, providing securement of the valve replacement. In embodiments, the clips envelope the native anterior and/or posterior leaflets of the native heart valve, mitral, or tricuspid for example. In embodiments, the clips wrap around the native leaflets to prevent the replacement heart valve from migrating into the atrium of the native heart. In embodiments, the clips are made of hypotube material comprising a hollow tubing (hypotube) material that has an inside diameter that mates with a wire of the helical wrapped wire of the frame of the valve replacement (including the frame body of the one-piece, or of the adapter of the two-piece systems).

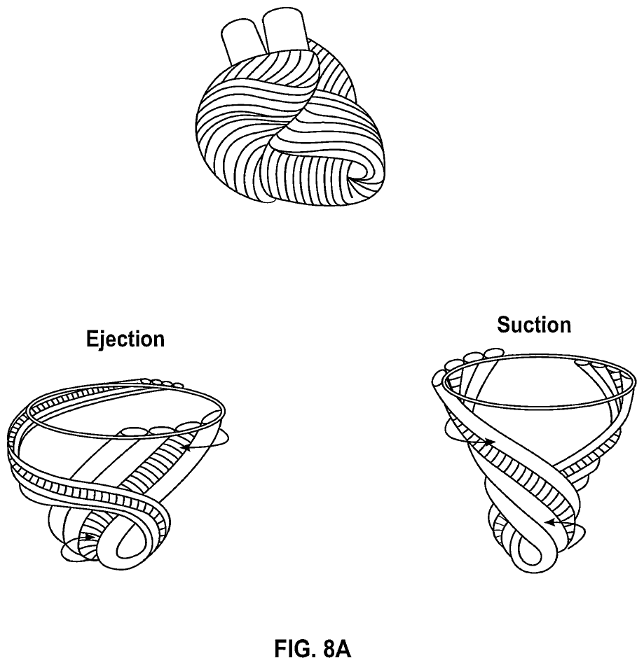

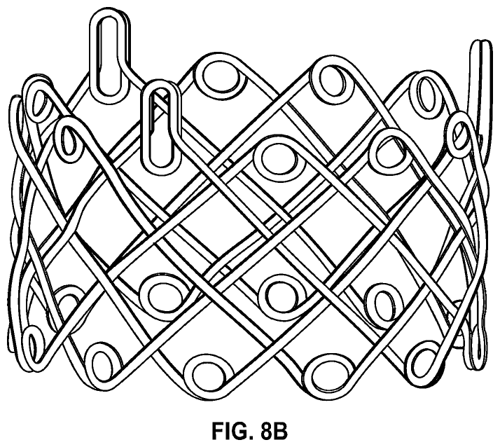

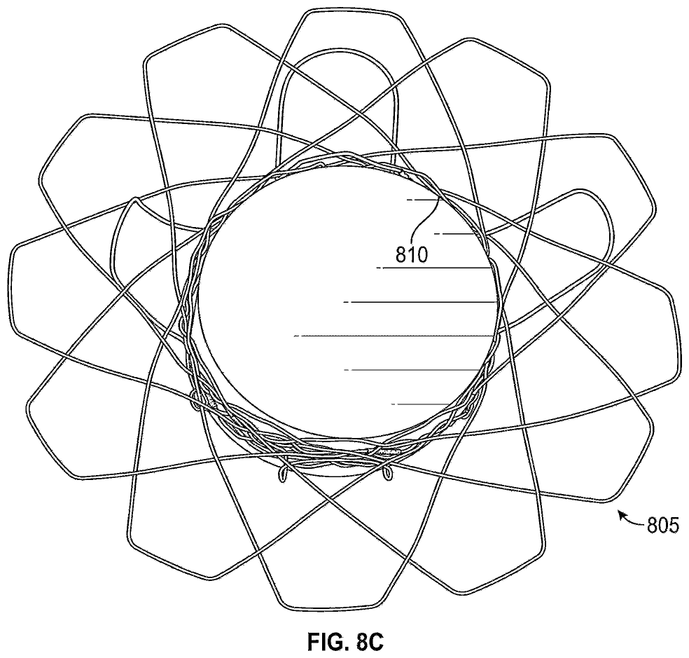

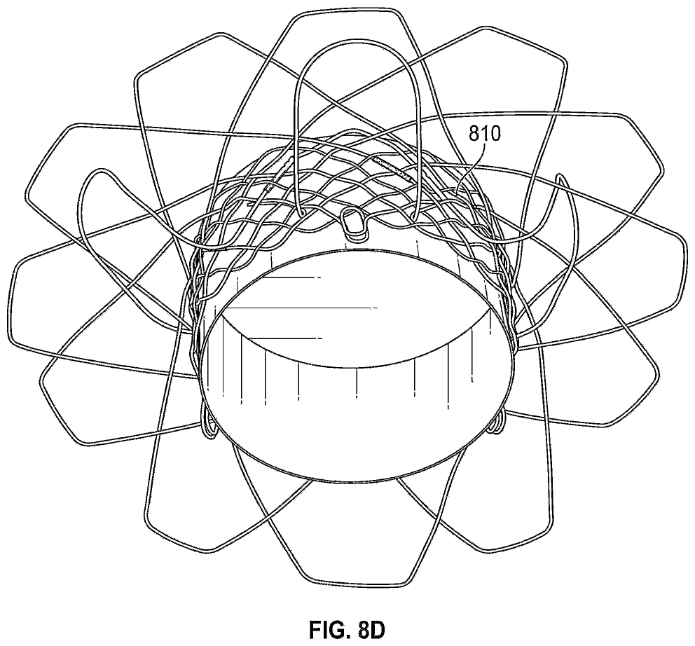

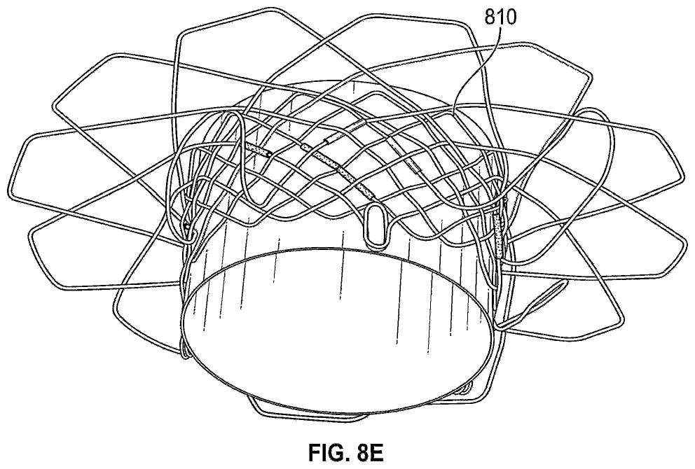

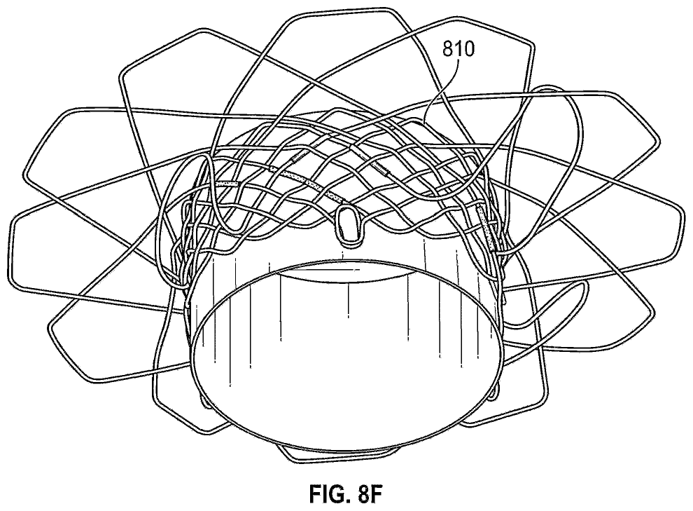

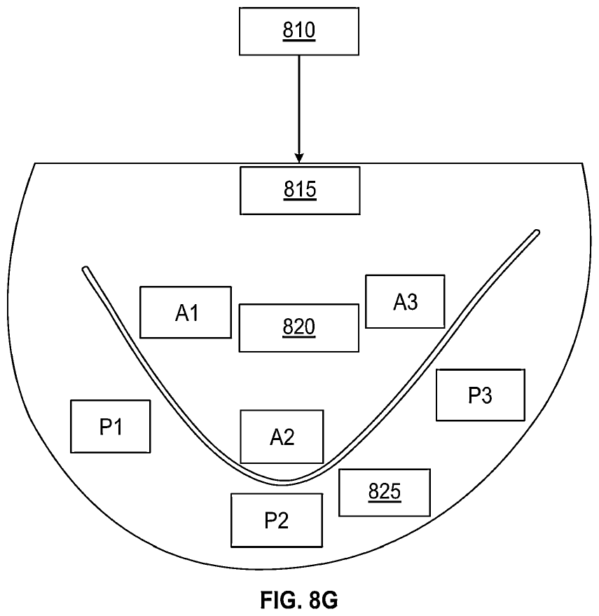

Various embodiments of the valve replacement may comprise various quantities of anchors at various angles and orientations. For example, one embodiment may comprise six anchors whereas another may comprise three anchors. In an embodiment comprising three anchors, applicable to the mitral valve, the valve replacement comprises an approximately 150° angle between the medial and lateral anchor struts with the A2 anchor (or A2 clip) anchoring to the anterior leaflet at or near the A2 region (as explained further with regard to FIG. 8G) of the anterior leaflet and the A2 anchor (or A2 clip) being symmetric between the two anchor struts. In another embodiment comprising three anchors, applicable to the tricuspid valve, the valve replacement comprises a uniform 120°/120°/120° spacing of the anchors (or clips). In another embodiment comprising three anchors, the valve replacement comprises an approximately 150° angle between the medial and lateral anchors with the P2 anchor (or P2 clip) anchoring to the posterior leaflet at or near the P2 region of the posterior leaflet and the P2 anchor (or P2 clip) being symmetric between the two anchor struts. Other angles and geometries are possible and within the scope of this disclosure.

In other embodiments, for smaller hearts, the valve replacement comprises an approximate 150° angle between an upper medial and lateral anchor struts with the A2 anchor (or A2 clip) being approximately symmetric between the two upper anchor struts, and an approximate 180° angle between a lower medial and lateral anchor struts with the P2 anchor (or P2 clip) being approximately symmetric between the two lower anchor struts. In other embodiments, for larger hearts, the valve replacement comprises an approximate 150° angle between an upper medial and lateral anchor struts with the A2 anchor (or A2 clip) being approximately symmetric between the two upper anchor struts, and an approximate 210° angle between a lower medial and lateral anchor struts with the P2 anchor (or P2 clip) being approximately symmetric between the two lower anchor struts.

The anchors (which include clips) may be made of the same wire as the braided frame or different wire—whether it be different in material and size. This provides a novel aspect: The ability to have thicker and/or more durable wire for the anchors allows for the anchors—which are required to attach to the valve tissue and maintain the valve replacement in place—to be stronger and/or firmer, without comprising the flexibility of the body frame. This enables the valve replacement to remain firmly and securely positioned within the heart valve while still allowing the valve replacement to move and function in accordance with the heart's natural movements.

Another novel aspect is the synchronization between the flanges and the anchors. Once implanted, the flanges provide a downward force on the heart tissue as the anchors provide an upward force. These two forces exerted by the valve replacement further secure it in place without compromising the fluidity of the braided body frame or the functionality of the leaflets.

### Helical Braided Design

The novel helical-braided designs of embodiments of the valve replacement purposefully leverage the natural helical movements of a beating human heart so as to balance both flexibility and strength. Studies of the human heart reveal that the mechanisms of ejection and suction are from a helical design of muscles in a “coil within a coil” formation, which are responsible for clockwise and counterclockwise rotation and functional activity. More specifically, the underlying anatomy of the human heart comprises a helical braid having a transverse basal loop of muscle for contraction that overlies an oblique helix that is responsible for ejection and suction within the heart.

The disclosed braided helical design is configured to put less stress on the individual components of the valve replacement because the valve replacement moves with the heart, i.e., the leaflets and anchors and other components have less stress and the valve replacement migrates less because its natural helical movement with the heart keeps it in place.

FIG. 8A generally illustrates the helical functionality of the human heart. As shown in FIG. 8A, the twisting and untwisting motions within the heart are created by inner helical spirals within the descending and ascending apical loop muscle segments, with the heart having a natural clockwise torsion/contraction for ejection and a natural counterclockwise loosening/lengthening for suction. In heart disease, the natural helix of the heart becomes architecturally altered in shape.

FIG. 8B generally illustrates an embodiment of the valve replacement as disclosed herein. As shown in FIG. 8B, an embodiment of the valve replacement comprises a helical braided design that mimics and reinforces the normal helical and elliptical formation of the heart and its twisting/turning motions. In some embodiments, the helical braided design may form a wire frame. In some examples, the helical braided design may be braided so as to allow the frame to move in several (e.g., three) directions. Relatedly, the braided design may in some examples allow the frame to accommodate movement (e.g., from simultaneous compression and twisting) along the longitudinal axis and axis of rotation. In one embodiment, the helical braided design comprises a design wherein the braided wires resemble a frame that may move and/or flex (e.g., symmetrically to a helical axis) as it is compressed and/or elongated around an open center. In some embodiments, the helical braided design may implement tensegrity and/or floating compression principles by, e.g., shape setting the wires and frame into predetermined formations (e.g., to allow wires to slide across each other in a non-rigid manner). For example, since principles may assist in decoupling movement in the axial and rotation directions such that the device can move in three dimensions to accommodate movement of longitudinal axis and rotation while heart is beating. By way of further example, such movement may be free in a constrained range, which range may be defined by the shape setting of the nitinol and the fabric sewn onto the frame, to permit movement of braided frame wires along one another at the over-under braids within a predetermined range of movement in one or more (or any) directions.

Both the one-piece system and the adapter and valve assembly of the two-piece system may comprise the helical braided design. A normal heart develops ejection and suction as a functional consequence of the contraction integrity of the apical ellipse. The braided helical design of the valve replacement maximizes shortening and lengthening of the heart muscles, thereby reinforcing the desired apical ellipse of a healthy heart movement.

For example, as the human heart muscles compress and descend, the braided helical wires of the valve replacement—rather than be stiff—also compress and descend with the heart muscles, thereby reinforcing a natural spiral compression and descension of the heart muscle surrounding the braided wires. With the braided helical design, the valve replacement conforms to and reinforces the natural movement of the heart. The braided helical design of the valve replacement produces a twisting spiral coil that develops torsion in a clockwise direction. And as the human-heart muscles lengthen and fill, the braided helical design reinforces a natural spiral lengthening and filling of the braided wires with the surrounding heart muscle, resulting in an untwisting spiral coil within the adapter or valve that develops an ejection force.

The novel braided helical design is significant for treating heart valves. By comprising a braided helical design, embodiments of the valve replacement reinforce the natural helical movement of the heart, and more naturally adapts and sits within the desired valve area. For example, embodiments of the valve replacement will tend to remain in the desired mitral or tricuspid valve area because the braided helical design will move (contract, twist and shorten, and untwist and lengthen) with the natural movements of the heart. This allows for the valve replacement to self-correct and seat within the valve area in a natural state, thus conforming to the heart's natural movements and encouraging central vortex flow.

The novel braided helical design thus facilitates a natural heart movement. In one embodiment, the valve replacement is held in place by the combined efforts of the flange and anchors, with the helical braided portion being in between the flange and anchors. The helical braided portion twists back and forth with the heart's natural movement, enabling a pumping-and-squeezing motion. The twisting motion, when the heart pumps, encourages flow of liquid through the valve replacement, thus allowing for better flow dynamics.

FIGS. 8C-F generally illustrate an embodiment of a valve replacement 805 as disclosed herein. The frame 810 of the valve replacement embodiment 805 may incorporate helical architecture using braided wire technology and fabrication. The frame 810 may utilize overlapping helical strands that conform to the heart's natural movements and encourage central vortex flow, as described above with regard to FIG. 8B. For example, the valve replacement 805 may not only facilitate contraction-like movements but also twisting, radial expansion, and other movements replicating movement of the heart.

The frame 810 may be made from braided wire. The properties of the frame, including densities and characteristics of the heart's anatomy, braiding design, wire thickness, etc., may facilitate not only the movements described above, but also accurize placement, maximize seal, and prevent migration, especially in coordination with an integrated and optimized anchoring system (and described in further detail below). In some embodiments, the frame 810 may be made from materials that include wires with determined thickness and geometry to designed to increase strength.

FIG. 8G shows different regions of heart tissue. For example, FIG. 8G shows a diagram of an aortic mitral curtain 815, an anterior leaflet 820, and a posterior leaflet 825. The anterior leaflet 820 may be slightly larger than the posterior leaflet 825, as is typical for most human hearts. The anterior and posterior leaflets 820, 825 may also be divided into separate areas labeled, e.g., A2 and P2.

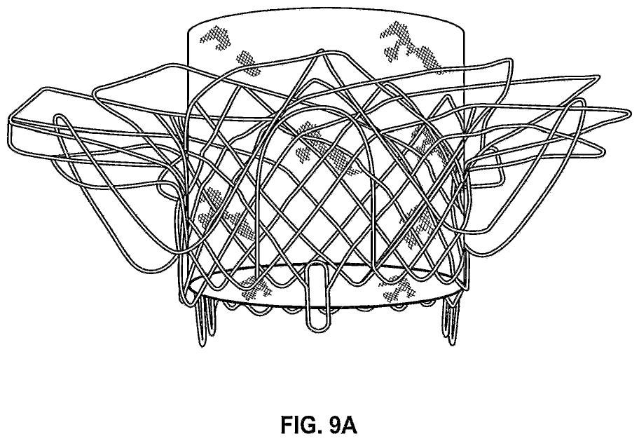

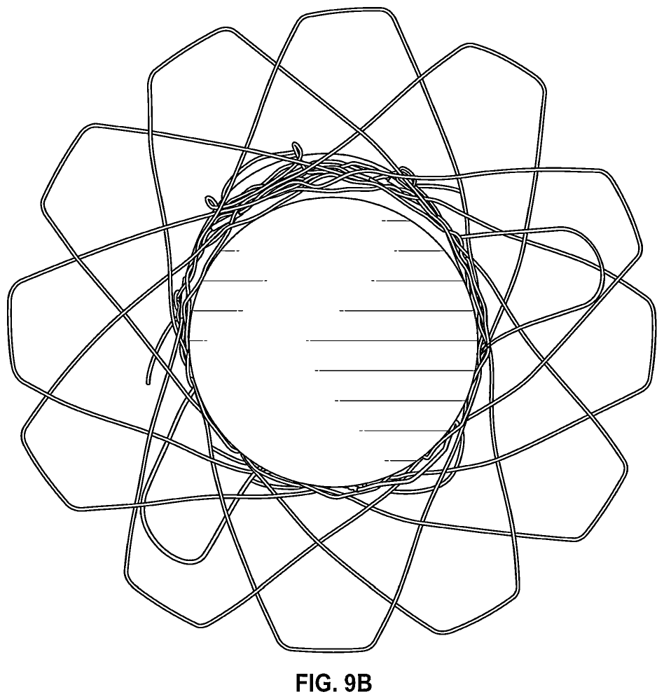

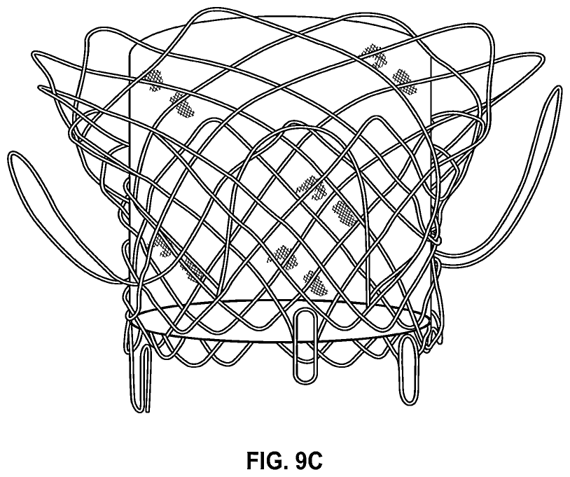

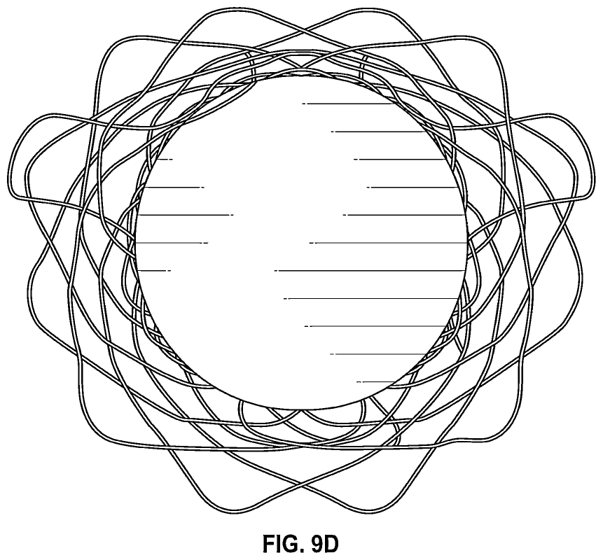

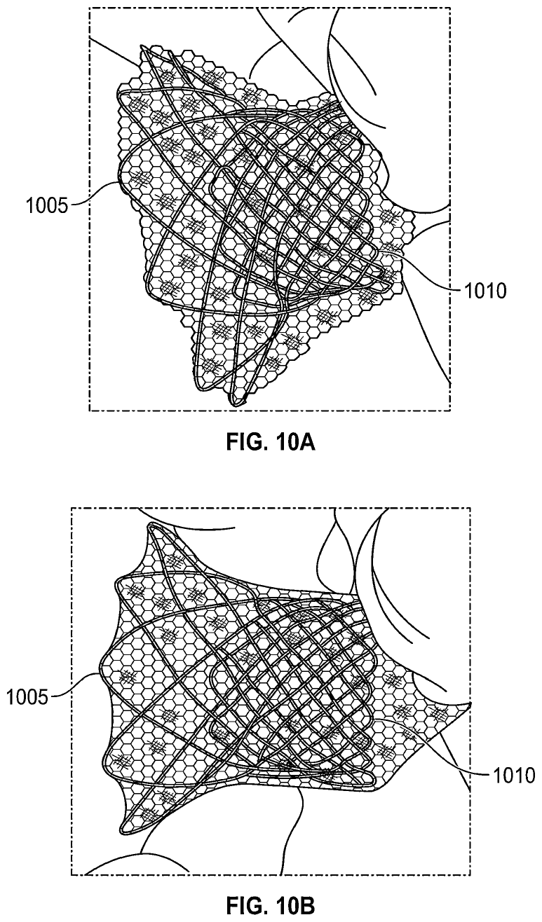

FIGS. 9A and 9B generally illustrate an embodiment of a valve replacement as disclosed herein, with FIG. 9B showing a vertical view of the embodiment shown in FIG. 9A. The valve replacement embodiment of FIGS. 9A and 9B may include a flange with a flatter surface. The flatter surface may be configured to rest on an annulus in an atrium area. In some examples, the exterior surface area of the valve replacement embodiment where the flange may meet a tubular adapter frame may be referred to for present purposes as a transition point, and may be similar to a right angle. In some cases, such a right angle may not conform to the surrounding native tissue such that it may leave empty space sub between the tissue and the valve replacement embodiment surfaces.

FIGS. 9C and 9D generally illustrate an embodiment of a valve replacement as disclosed herein, with FIG. 9C showing a vertical view of the embodiment shown in FIG. 9D. The valve replacement embodiment of FIGS. 9C and 9D may include a flange with a more curved surface. For example, and more specifically, the transition point of the valve replacement embodiment of FIGS. 9C and 9D may also be more curved (in contrast to a right angle), which design may assist in the surfaces of the valve replacement embodiment to expand into spaces between the surfaces and the surrounding native tissue.

### Material Covering

In some embodiments, different materials are prepared prior to assembling into a continuous covering. In other embodiments, material may be added and receive a modification treatment post-assembly that is applied to only specific locations on the valve replacement.

In some valve replacement embodiments disclosed herein, tissue attachment and ingrowth may be promoted in an area that is desired to become anchored to the tissue, while cellular interaction can be limited to simple endothelialization or no response at all, to allow disturbance of part of the device at a later date without risk of tissue or thrombus embolization. Put simply, the varying material used may be either conducive or non-conducive to chemical bonding. For example, in a preferred embodiment, the materials in contact between the inner portion of the adapter and the outer portion of the valve assembly do not bond, so as to allow for movement of both portions; whereas the material on the outside of the adapter bonds with human tissue. Thus, depending on the location, materials may be used such that cellular growth is inhibited or promoted.

Some valve replacement embodiments may be encased, either completely or partially, in a continuous material covering to elicit the type of physiological response that is desired as well as the mechanical behavior. Though the covering is continuous—as in there are no material gaps at the transitions of physical features—the materials may be modified locally in areas of the device to behave differently. For example, the material covering one side of the flange may be deliberately nonporous to facilitate sealing, while the material on the other side of the flange may be a knit that facilitates tissue ingrowth for anchoring. Alternatively, the flange could be alternating rings of nonporous and ingrowth material on both sides of the flange. These techniques can be applied to any surface of the device.

Material differences range from being entirely different materials—natural tissue or synthetic fabric—to physical and chemical surface modification, to obtain the desired mechanical and biocompatible properties. These modifications can include but are not limited to coating, etching, mechanically biasing, ion infusion, various deposition techniques, and oxidizing/nitriding/carbiding. Modifications may be used in any combination to achieve the desired result.

FIGS. 10A and 10B generally illustrate embodiments of a valve replacement as disclosed herein. As shown in FIGS. 10A and 10B, an embodiment of the valve replacement may comprise a continuous piece of material around the outside of the frame. A continuous seal may be configured from the material (such as fabric) extending from an inflow edge 1005 of the valve replacement to the extrados 1010 of the body of the valve replacement. A strip of ingrowth fabric may be sewn around the inflow edge of the valve replacement, with a non-porous coating forming a continuous seal extending into the ventricle. Material can also be configured to fill spaces between the inner and outer fabric. For example, in an embodiment, filler fabric is selectively placed within the valve replacement to fill in spaces and gaps between the materials or areas where the frame and fabric have gaps.

The continuous surface of the fabric may be locally influenced and characterized for modulating or even contradicting properties, such as coating with medical polymer in locations where no tissue attachment is desired, hydrogels where space-filling or latent actions are desired, or a hydrophilic tissue adhesive. The continuous material structure of the fabric may be voluminous in nature, filling space and adapting the round heart valve to the asymmetrical shape of the valve annulus. Combined with other attachment methods, an embodiment of the mitral-valve adapter fabricated with this method aids in engagement and attachment of the leaflet tissue and other sub-valvular structures. The partially porous fabric provides an improved seal for a replacement valve, enabling accommodation to irregular shaped anatomy through the compliance of the fabric. In embodiments, fabric is selectively treated by partial dipping in a coating that provides additional properties to the fabric, such as improved scaling.

In other embodiments, the valve replacement may be fabricated using a constraint to hold the valve replacement at a specific dimension while attaching material to influence device performance. A fabrication technique is disclosed, which acts to influence the disposition of a braided wire frame-removing the inherent freedom of movement and unpredictability that is present between relative members of the frame structure when in a load-free state. This technique involves restraining the radial expansion of the frame with a constraint, such as feeding some number of sutures through or around the structure to hold it at a specific dimension other than its unrestrained, “free” dimension. In subsequent fabrication steps, the structure is incorporated into an assembly that adopts this new configuration and considers this to be the final dimension. When the constraints are removed from the braided frame, this braided frame tries to recover to its original “free” dimension—applying additional radial force to the surrounding structure while being constrained to the desired dimension.

The degree of radial force transmitted to the fabric material from the frame can be adjusted as required to achieve the optimal combination or performance properties. In particular, the strain energy density of the structure can be more uniform. A greater stiffness is achieved (resulting in a better seal) with less material, resulting in a more low-profile structure. The suture finally provides a biasing of the structure toward a desirable diameter and height for the valve structure.

To expand the concept further, structures that possess features described herein may be co-deployed singularly or with a connected design, so as to engage both the mitral and the aortic valve apparatus and/or annulus. The intent is to influence the leaflets of both valves, as well as the angulation of the valves relative to one another, to ensure the most effective management of flow through the ventricle and maximizing the efficiency of the outflow tract.

In some embodiments, the valve replacement is covered in a material that wraps around the frame in a continuous manner. Embodiments of the material are fabric and animal tissue. By using materials that can be locally modified to change characteristics such as porosity and surface roughness, a certain level of control over cellular interaction on the various parts of the device can be achieved. In other embodiments, the adapter body and atrial flange may be covered in fabric for the purpose of flow scaling and/or influencing (e.g., either promoting or inhibiting) tissue growth after implantation.

The material used further assists with the loading and deployment of the valve replacement. For example, the material may promote the valve replacement to function as a re-valve system, wherein a tubular braided fabric tube (coated with a polymer to decrease porosity to blood) surrounds the frame and constrains the diameter. This tube is sewn onto the frame, sometimes in conjunction with a leaflet panel, so that the strings can be removed and what remains is a pretensioned frame constrained by the fabric. In embodiments, an elastomer-coated, tubular knit, shape-set fabric is attached to the braided frame. In embodiments, dip-coated braided frames are utilized, such as a frame dipped in urethane, either as a whole device or partial. In other embodiments, treated panels are sewn onto the braded frames. In other embodiments, sections of treated fabric are cut into panels that are configured to be able to be sewn onto a at least partially circular surface, such as a flange.

### Sewing Methods and Belt Loops

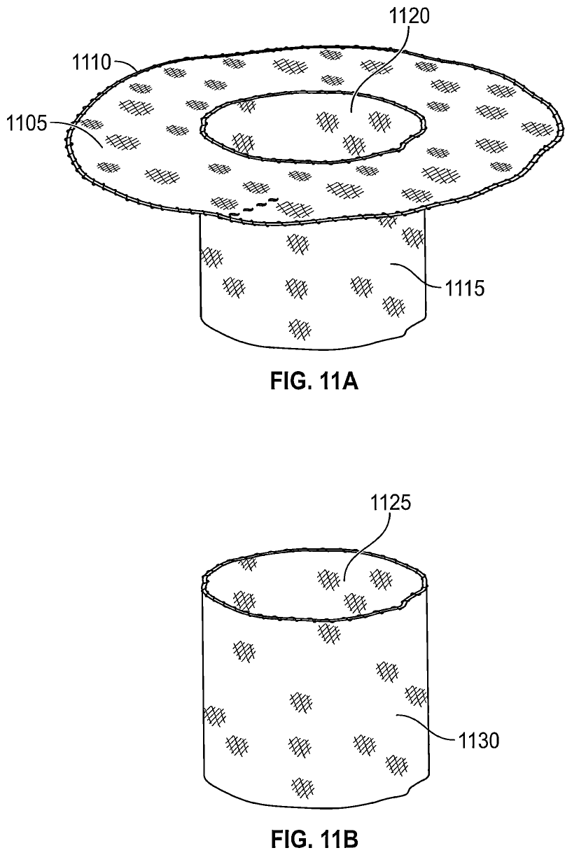

FIGS. 11A and 11B generally illustrate embodiments of the valve replacement as disclosed herein. More specifically, FIGS. 11A and 11B show various embodiments of the fabrication of material for the valve replacement, focused on the two-piece system, and is also consistent with a one-piece system as well. In embodiments, material for the various parts may be made of the same or different materials, respectively. Such materials may include, for example, a textured or hard-knitted style fabric or textiles, or bioresorbable mesh (e.g., made from polymers), elastomeric scaffolds, or other materials. Other materials with similar or different properties may be used, such as permeable or impermeable materials, with various degrees of elasticity, and stretchable in predetermined directions or any direction, and biocompatible materials.

FIG. 11A shows a flange outer material 1105, a flange rim material 1110, an outer adapter cuff material 1115, and an inner adapter cuff material 1120 of the valve replacement. The flange outer material 1105 may form a circular top piece, wherein it may further have a coating to reduce fluid permeability. The flange rim material 1110 may form a circular bottom piece that stretches to allow expansion of the valve replacement when deployed. The outer adapter cuff material 1115 may constitute a cross-stitch that attaches the ends to create the tube-like shape. The inner adapter cuff material 1120 may comprise a running or cross-stitch that attaches the ends to create a tube shape, wherein the fabric of the inner adapter cuff material 1120 may have limited elasticity and either stitch maintains the integrity of the inner adapter cuff material 1120.

In one embodiment, the flange rim material 1110 and the outer adapter cuff material 1115 may be combined, either by cross-stitching or other method known to one skilled in the art, to form a secant bottom piece. The flange may be stitched furthest away from any anchor slots. The formed secant bottom piece may be positioned onto the bottom of the adapter or the one-piece system, wherein the shape-set thread around the body of the frame is cut and wrap-stitch is used to close or secure the anchor slots.

In another embodiment, the inner adapter cuff material 1120 may be attached to the inner opening of the flange outer material 1105 to form a top piece of material. The top piece or material may be slid over the top of the adapter or the one-piece system after which the fabric is wrap-stitched closest to the frame.

In another embodiment, the bottom piece of material and the top piece of material may be connected, such as by wrap-stitching at the bottom of the frame to connect the top piece of material to the bottom piece of material. For this, the fabric of the flange outer material 1105 and the flange rim material 1110 may be smoothed and held in place (such as with sewing clips) and connected around the wire flange (such as with a running stitch), wherein the border of the flange may be circular and not rigid. The excess fabric may be trimmed, and additional stitching may be added around the flange and each wire flange tip. Additional stitching may be performed along the wires to secure the fabrics together and keep flush against the wire flange. The stitching may be done to the second crossing of wires, followed to the next wire, and then up towards the tip of the wire flange. This process may be continued around the flange and repeated on the next set of wires.

One embodiment of the outer adapter cuff material 1115 may be folded in half and secured together (such as with a sewing clip), after which a blanket stitch may be performed around the edges. The blanket stitch allows the outer adapter cuff material 1115 to retain its shape without sinching the fabric. The outer adapter cuff material 1115 may be turned inside out and placed over the anchor wire, after which the outer adapter cuff material 1115 may be secured to the front and back of the anchor wire (such as with a running stitch). In this, the seam of the stitch (used to close the anchor slots) may be caught between the front and back fabric of the outer adapter cuff material 1115 to secure it to the adapter or one-piece system. The running stitch may encompass the front of the outer adapter cuff material 1115, the seam, and the back of the outer adapter cuff material 1115 along the base of the anchor wire. Once the outer adapter cuff material 1115 is secured at the base, a stitch may be continued along the anchor wire to keep the outer adapter cuff material 1115 from slipping or sliding on the anchor. Fabric may be slightly caught, wherein it is not loose enough to leave excess fabric but not tight enough to affect the shape of the wire.

In embodiments, stitching of the fabric to the replacement valve helical braided wire architecture performs several functions, including the following: sewing of fabric to the braided wire architecture helps constrain the braided wire of the replacement valve from migrating into the ventricle or atrium and helps prevent paravalvular leaks, while at the same time permitting slight movements along the unfixed nodes at the over-under braids to allow the replacement valve to move with the natural helical movements of the heart.

FIG. 11B shows an inner valve cuff material 1125 and an outer adapter cuff material 1130. In one embodiment, leaflets may be connected to the inner valve cuff material 1125, such as along a strip of fabric with a double running stitch along the belly of the leaflet, wherein the stitches are uniform across the leaflets to allow for proper valve opening and closing. The leaflet or commissure tabs may be exposed, such as by laser-cutting with slots at the top of the inner valve cuff material 1125 (wherein a strip of the inner valve cuff material 1125 may be folded in half, making sure that leaflets are aligned on top of each other; and wherein the ends of the inner valve cuff material 1125 may be attached to the junction of the belly and tabs with a double running stitch). Following the exposure of the leaflet tabs, the inner valve cuff material 1125 may be placed inside the valve assembly frame, the tabs may be pulled through commissure wires and laid flat between the leaflets and the inner valve cuff material 1125.

In one embodiment, the ends of the outer valve cuff material 1130 may be connected and the outer valve cuff material 1130 slid over the outside of the valve assembly frame.

In another embodiment, the inner valve cuff material 1125 and the outer valve cuff material 1130 may be connected together. After placing the inner valve cuff material 1125 on the inside of the valve assembly frame and the outer valve cuff material 1130 on the outside of the valve assembly frame, the two parts may be connected to the bottom of the frame (such as by tacking down both parts with a square knot) directly below commissure wires, and the parts may be stitched along the bottom of the frame. Following a commissure attachment, which is set forth in the following paragraph, both portions may be combined by sewing through the frame and the top of the valve assembly frame may be stitched. Additional steps may comprise, along the upper perimeter of the valve, stitching around the wires travelling from the commissures downwards and away from the peaks so as to create a z-shaped pattern. In this, the stitches may connect the inner fabric behind the leaflet and the outer valve cuff material 1130.

In an embodiment of a commissure attachment, leaflet tabs are fed through the commissures, wherein each tab folds towards its own leaflet and is wrapped around the commissure wires. The ends of the tabs may be held together against the entry of the tabs and secured together, such as with running stitches vertically and on the inside of the valve assembly. Stitching may continue in front and around the commissure, such as for 3-4 times, and entering and exiting at the location of the running stitch. Stitches may be perpendicular, comprising of embodiments such as a running stitch along the y-axis and a wrap-stitch along the x-axis.

In other embodiments of the fabrication of material for the valve replacement wherein the focus is on the one-piece system, a fabric for the various portions may comprise a stretchy and semi-transparent fabric; wherein the outer adapter cuff material 1115 and flange rim material 1110 may be sewn together to create the outer piece. Cross-stitch may be used to connect the edges of the outer adapter cuff material 1115 and to connect the outer adapter cuff material 1115 to the flange rim material 1110.

In a separate embodiment, a fabric for the portions comprises an inflexible and opaquer fabric where the coating is visible; wherein the inner adapter cuff material 1120 and flange outer material 1105 may be sewn together to create the inner piece. A running stitch may be used to connect the edges of the tubular inner adapter cuff material 1120 and a cross-stitch is used to connect the inner adapter cuff material 1120 to the flange outer material 1105.

For these embodiments focused on the one-piece system, an inner valve cuff material may be created, wherein leaflets are attached to the inner adapter cuff material 1120 and wherein leaflet tabs are placed through slots of the inner adapter cuff material 1120 where the junction of the inner adapter cuff material 1120 and tabs meet, with the leaflets held in place, such as with sewing clips. Using a double running stitch, the belly's edge of each leaflet is sewed to the inner adapter cuff material 1120. The tabs and top edge of the leaflet(s) are flushed and level with one another and the running stitch on each belly of the leaflet(s) is level and uniform. (Inconsistent stitches can lead to a defective valve.) After the leaflets are attached to the inner adapter cuff material 1120, the inner adapter cuff material 1120 is folded in half, keeping leaflets level and held in place. A double running stitch may then be sewn directly down from the junction of the leaflet tabs, continuing away from the leaflets with a running stitch back up towards the junction of the leaflet tabs. (The running stitch should be away from the leaflet belly.) Following these steps, the inner valve cuff material should create a tube.

A first set of materials may be created by connecting the inner valve cuff material from above to the flange outer material 1105, such as with a cross-stitch, wherein leaflets are away from the scam.

Once the first set of materials is created, it may be connected to a second set (e.g., the flange rim material 1110 and outer adapter cuff 1115 previously sewn together) by using a running stitch through the frame (between the double running stitch of the leaflet belly) and following the belly stitch of the leaflet(s) to secure both sets together. The tabs are then secured through the commissures and wrap-stich is used to connect the second set to the first set at the base of the frame. The deployment apertures are created, by cutting the fabric, before finishing the wrap-stich. Once connected, a beta-stitch is incorporated on the flange and anchors' cuff placements.

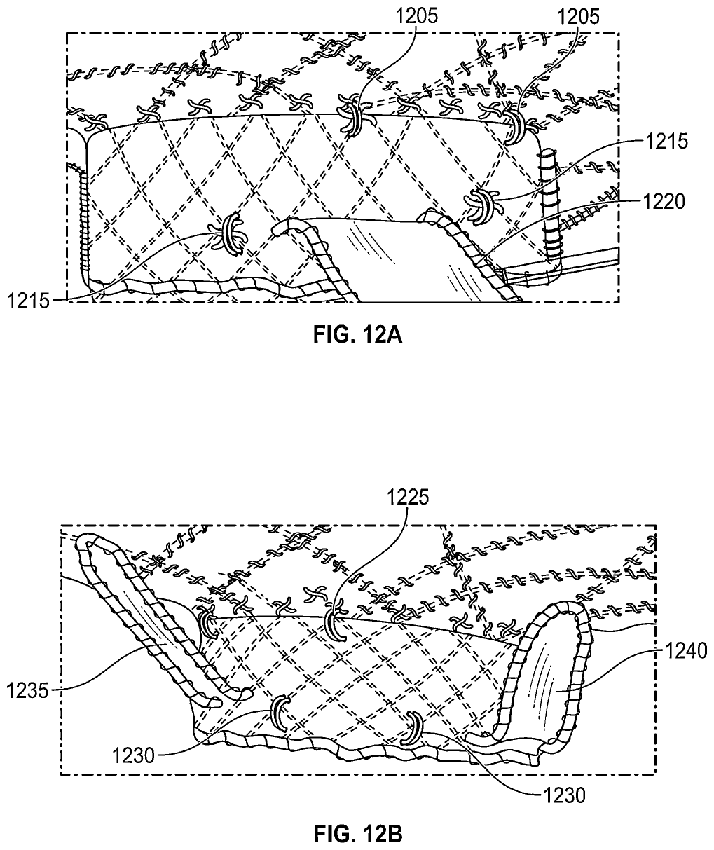

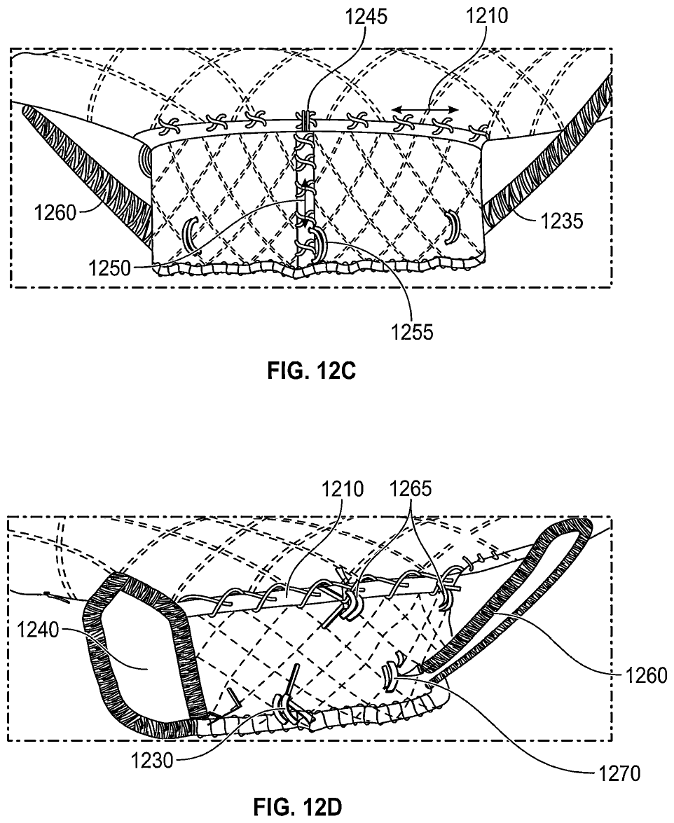

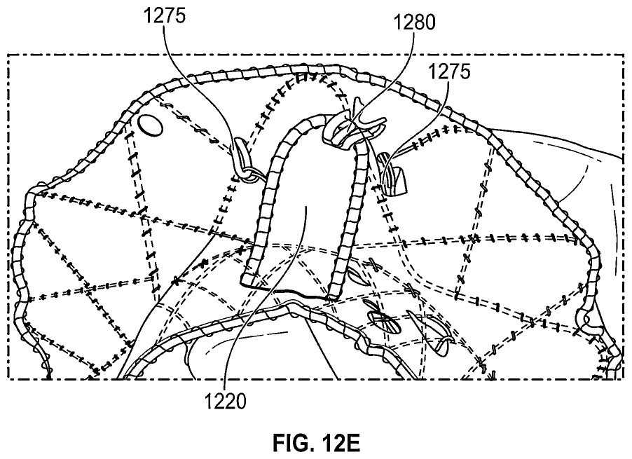

FIGS. 12A-12E generally illustrate embodiments of the valve replacement as disclosed herein. More specifically, FIGS. 12A-12E illustrate deployment belt loops commonly used for the two-piece system. The deployment belt loops may comprise one or more sets of loops.

In a preferred embodiment, the adapter comprises seven belt loops at the base of the body of the adapter, wherein there are belt loops on either side of three anchors and one belt loop at a vertical seam. The adapter further comprises five belt loops at the horizontal seam between the body and the flange, wherein the five belt loops are located at crosswires on the same plane as one another. Hidden belt loops may be found behind the long anchors (P1/P3 anchors). The adapter also comprises four belt loops along the flange, located at the crossed wires to the left and right of the long anchors. Additionally, the adapter comprises two belt loops at the tips of the long anchors. Though in a preferred embodiment the anchor-tip belt loops comprise three loops and all other belt loops comprise two loops, it should be known that the belt loops are not limited to a specific number of loops.

FIG. 12A shows the belt loops in relation to the P1 and P3 anchors. The adapter comprises one or more belt loops 1205 behind (i.e., on the body and hidden from view unless the anchor is lifted up) the P1 and/or P3 anchor 1220 and along the horizontal scam 1210. And the adapter comprises one or more belt loops 1215 to the left and right of the P1 and/or P3 anchor 1220.

FIG. 12B shows the belt loops in between the P1 1235 and P2 anchors 1240. Belt loops 1225 along the horizontal seam 1210 are at the same plane. The adapter further comprises belt loops 1230 to the side of the P1 anchor 1235 and the P2 anchor 1240, wherein the belt loops 1230 are also approximately at the same plane.

FIG. 12C shows the belt loops in between the P1 1235 and P3 anchors 1260. One belt loop 1245 is at the junction of the horizontal 1210 and vertical seam 1250 and one belt loop 1255 is at the bottom of the vertical scam 1250.

FIG. 12D shows the belt loops in between the P2 1240 and P3 anchors 1260. The belt loops 1265 along the horizontal seam 1210 are at the same plane. Because the P3 anchor 1260 is slightly higher, the belt loop 1270 near the P3 anchor 1260 is adjusted to a height approximate to the P3 anchor 1260. The belt loop 1230 is in a position in relation to the location of the P2 anchor 1240. In a preferred embodiment, the anchors are biased towards the flange. In other embodiments, the anchors may be perpendicular to the body.

FIG. 12E shows anchor loops in relation to the P1 and/or P3 anchors. In one embodiment, the anchor loops comprise double loops 1275 positioned at the crossed wires on the flange to the left and right of the P1 and/or P3 anchor 1220, and the anchor loops further comprise a triple loop 1280 at the tip of the P1 and/or P3 anchor 1220.

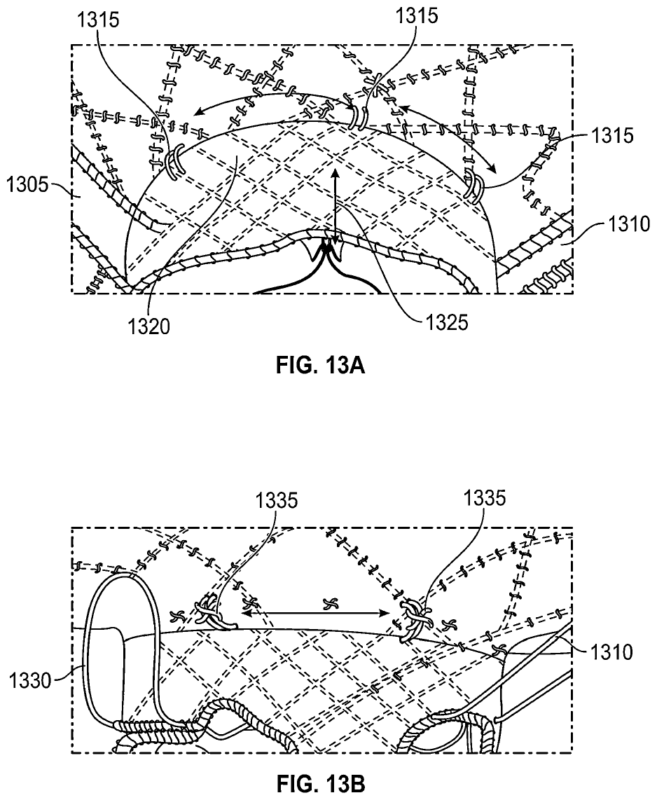

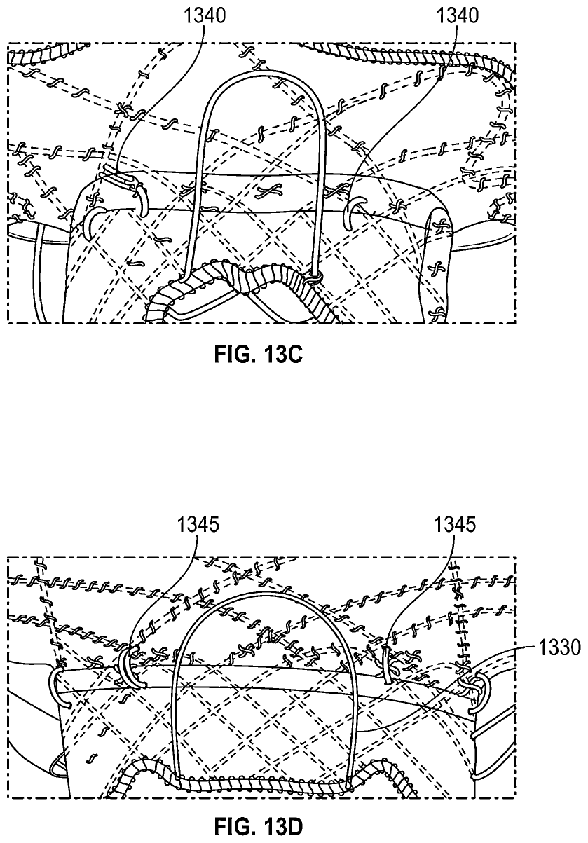

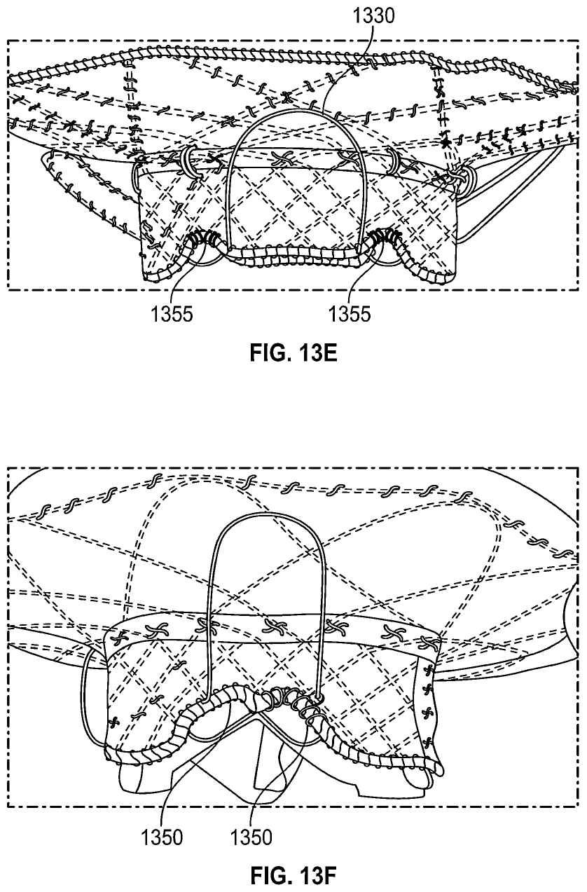

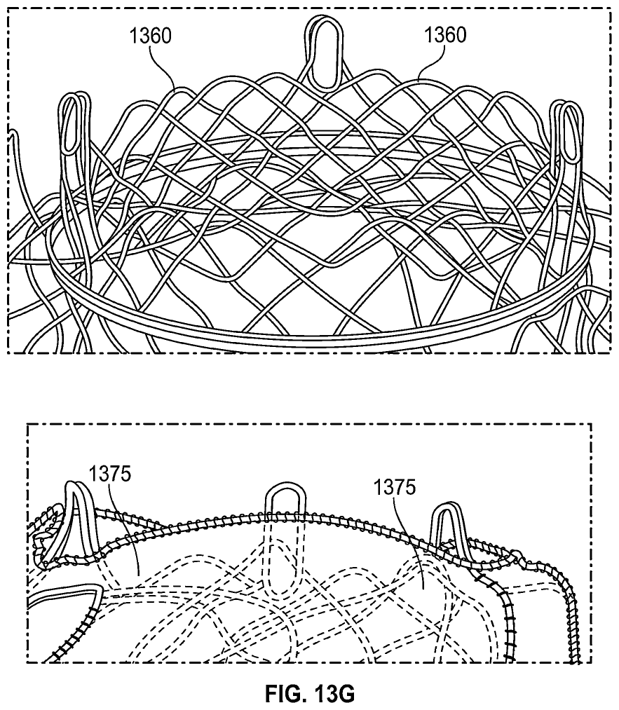

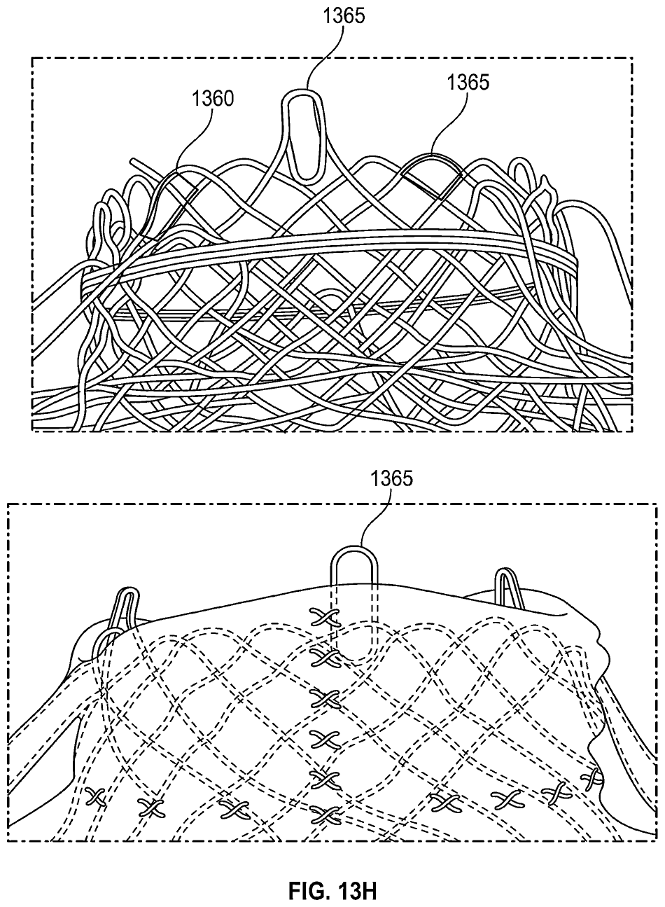

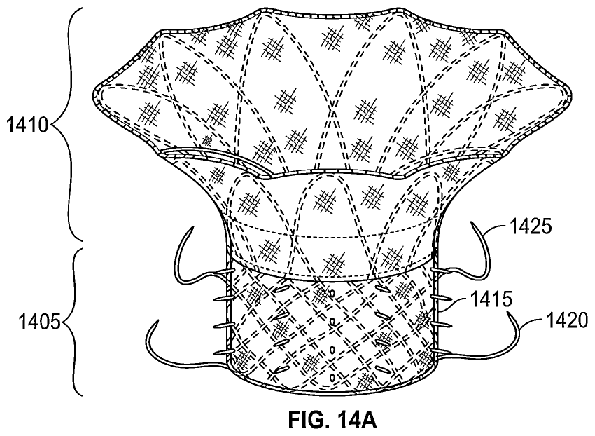

FIGS. 13A-13D generally illustrate embodiments of a valve replacement as disclosed herein. FIGS. 13A-13D illustrate deployment belt loops commonly used for the one-piece system. The deployment belt loops may comprise one or more sets of loops.

In a preferred embodiment, the one-piece system comprises seven belt loops, wherein the belt loops on the adapter body of the one-piece system are double loops and the belt loops located on a horizontal scam are at the crossed wires along the same plane.

FIG. 13A shows belt loops in between the P1 1305 and P3 anchors 1310. Three belt loops 1315 are located along the horizontal scam 1320 with one belt loop located at the junction of where the horizontal 1320 and vertical seams 1325 meet, and located in line with a commissure wire. The two other belt loops are located just outside of the P1 1305 and P3 anchors 1310.

FIG. 13B shows belt loops in between the P2 anchor 1330 and either the P1 1305 or P3 anchor 1310. Two loops 1335 are located along the horizontal seam and secured at the same plane of the crossed wires outside the P2 anchor 1330 and the long anchor (P1/P3 anchor).

FIG. 13C shows the belt loops 1340 of the long anchor, wherein the belt loops 1340 are located on either side of the long anchor.

FIG. 13D shows the belt loops 1345 of the P2 anchor 1330, wherein the belt loops 1345 are located on either side of the P2 anchor 1330.

FIGS. 13E and 13F show an embodiment of the location of deployment apertures, wherein the deployment apertures 1350 are located directly below each long anchor and are located on either side 1355 of the P2 anchor 1330.

FIGS. 13G and 13H show another embodiment of the location of deployment apertures 1375. In this embodiment, fabric is removed (e.g., in a triangular shape) two peaks away 1360 from the commissures (or commissure posts) 1365 so as to expose the wire peak. A wrap-stich 1370 is placed around the perimeter of the frame so as to catch wires around the exposed peaks.

### Engagement Structures

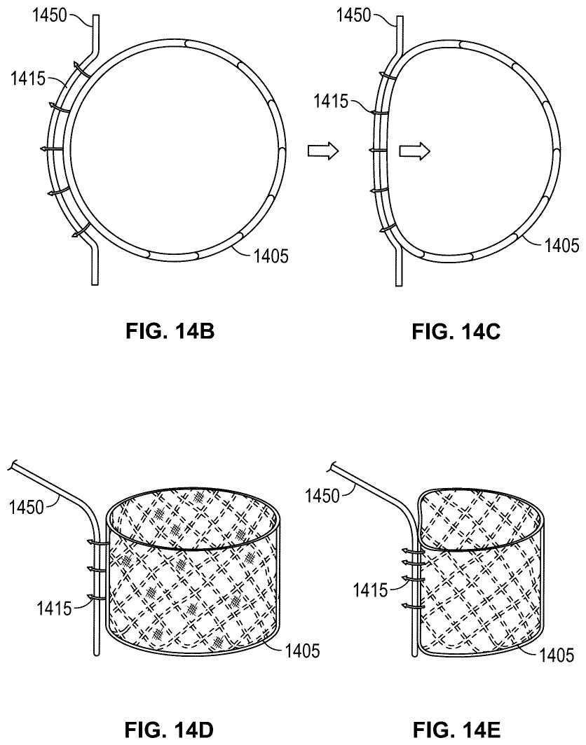

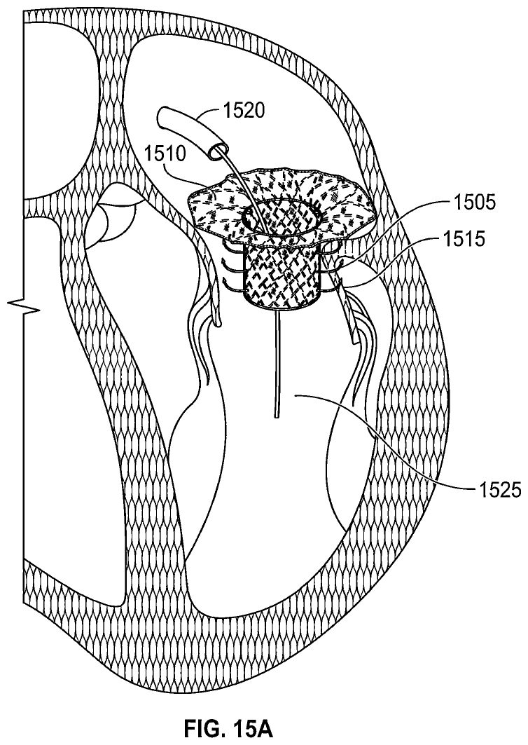

FIG. 14A generally illustrates an embodiment of the valve replacement as disclosed herein. In an embodiment, as shown in FIG. 14A, the adapter or the one-piece system comprises a body 1405 and an atrial sealing skirt 1410 (which in some aspects may be a flange). The adapter body 1405 and sealing skirt 1410 may be constructed of varying material and vary in dimensions. For example, the adapter body 1405 and sealing skirt 1410 may be made up of a wire braid of one or more wires with different diameters. The wire may be made of material such as nitinol and be designed to be compressed to a small diameter—such as 4 mm to 6 mm—to be delivered in a catheter. When released, the adapter body 1405 and sealing skirt 1410 may expand in size (i.e., the body expanding to 25 mm or greater in diameter and the sealing skirt expanding anywhere from 40 mm to 70 mm in diameter).

The exterior surface of the adapter body 1405 may also be covered with a multitude of small, short barbs 1415. The barbs 1415 may be used to engage the leaflet or annulus of a malfunctioning cardiac valve, such as a mitral valve. The barbs 1415 may be made up of basic, short wires and/or may also have an extra barb-component, like a fishhook barb, to fixedly retain the annular tissue.

The adapter body 1405 may also have one or more hooks 1420 or 1425 (more or less in number than the barbs 1415) varying in size, that can hook under the native valve tissue. These larger hooks may or may not have fishhook barbs. The larger hooks may have a spring-like function that engages with the native valve tissue and prevents it from moving.

In a preferred embodiment, the sealing skirt 1410 may be connected to a catheter, wherein the adapter Attachment is sequentially released from the catheter once the adapter body 1405 is released and engaged with annulus tissue. The sealing skirt 1410 may be designed to flex downward, toward, or even past the plane defining the joint between the adapter body 1405 and the scaling skirt 1410—so as to be radially overlapping with the adapter body 1405. The multitude of barbs 1415 on the adapter body 1405 would work together to ensure the adapter body 1405 is strongly engaged in the native annulus and resists the downward pressure of the sealing skirt 1410, such that the scaling skirt 1410 would create a strong seal against the atrial tissue surrounding the native valve annulus.

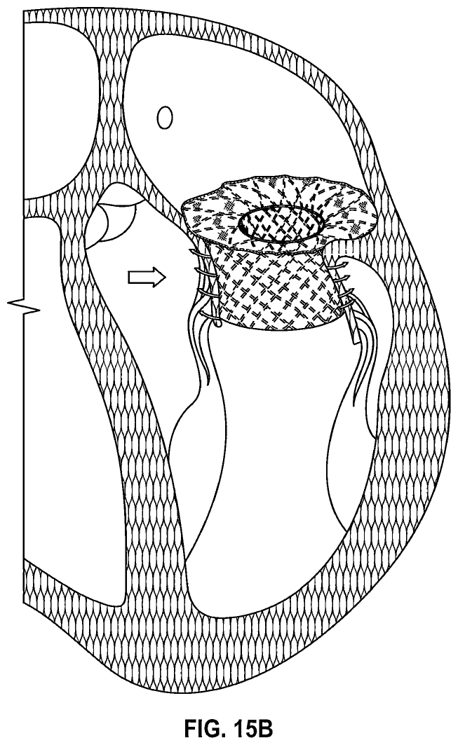

FIGS. 14B-14E generally illustrate embodiments of the valve replacement as disclosed herein. In these figures, the sealing skirt is not shown for case of illustration. As shown in FIG. 14B, the adapter body 1405 is designed with a braid of varying weave densities and/or wire diameters, and/or combined with releasable mechanisms such that the adapter body initially has a round cross-section. The adapter body 1405 has barbs 1415 designed to engage a native valve leaflet 1450. Once the barbs 1410 are engaged, the anchoring/attaching functionalities of the adapter Attachment cause it to conform to a “D-shape” or other asymmetrical shape-keeping the adapter body 1405 cylindrical or otherwise specifically shaped to receive the valve structure. This accommodation and conformity is achieved via the different weave, wire diameters, or mechanism enabling such. As shown in FIG. 14C, the change in shape creates a sharper curve radius to make the D-shape. The change from a circular cross-section to a D-shape cross-section may pull the leaflet, which can be useful, for example, in a mitral valve where an implant such as the adapter body may cause outflow tract obstruction. FIGS. 14D and 14E disclose an oblique view of the structure and mechanism corresponding with FIGS. 14B and 14C. Embodiments disclosed in FIGS. 14B-14E may also comprise the sealing skirt and other features described in previous drawings. FIGS. 15A and 15B generally illustrate embodiments of the valve replacement as disclosed herein. FIG. 15A discloses an embodiment of the valve replacement implanted in a malfunctioning mitral valve, with the body 1505 deployed in the mitral valve and the sealing skirt 1510 deployed against the floor of the left atrium. In this embodiment, the adapter body 1505 is oriented at a slight angle (i.e., from 10-30 degrees relative to the plane of the skirt), such that when deployed, the adapter body 1505 is biased towards the posterior leaflet 1515.

Deployment as disclosed in FIG. 15A ensures good engagement of barbs into the posterior leaflet but not necessarily the anterior leaflet. The system may be designed to normally be in this geometric condition but be mechanically expandable by design so that it can expand to engage the anterior leaflet, then released back to the normal position after the barbs and/or hooks engage the anterior leaflet. This forces the anterior leaflet towards the posterior leaflet and away from the left ventricular outflow tract obstruction (LVOT) ensuring it is not obstructed post-procedure. Also shown are the delivery catheter 1520 and guidewire 1525.

In another embodiment, the body of the valve replacement may be used to engage the leaflets with the barbs, wherein the body expands to a diameter larger than the diameter at deployment to ensure engagement with the leaflets. As the device is further deployed, the diameter of the engaged portion reduces to a final configuration—symmetrical or asymmetrical—thereby pulling the leaflets towards the device and away from the LVOT.

FIG. 15B generally illustrates an embodiment of an adapter attachment as disclosed herein. FIG. 15B discloses a final configuration of an adapter in its original position after release, wherein the anterior leaflet is drawn and held towards the posterior leaflet, ensuring no obstruction of the LVOT.

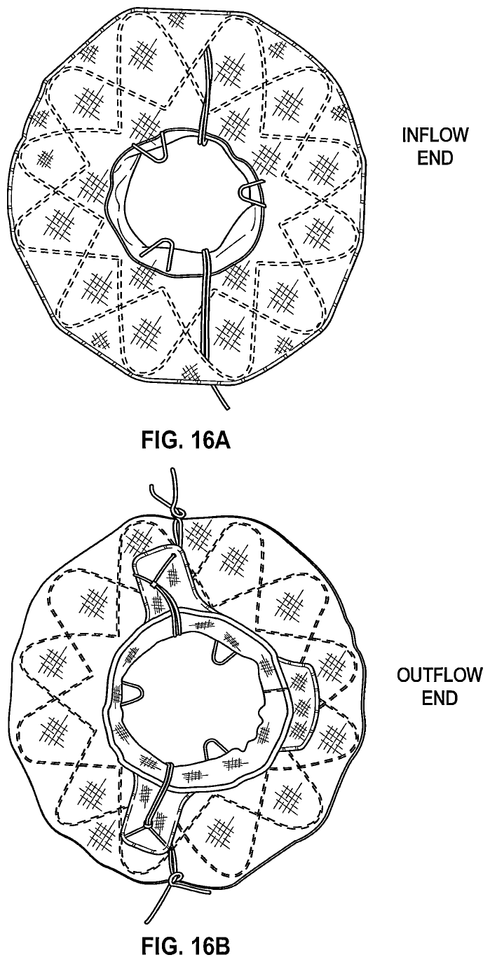

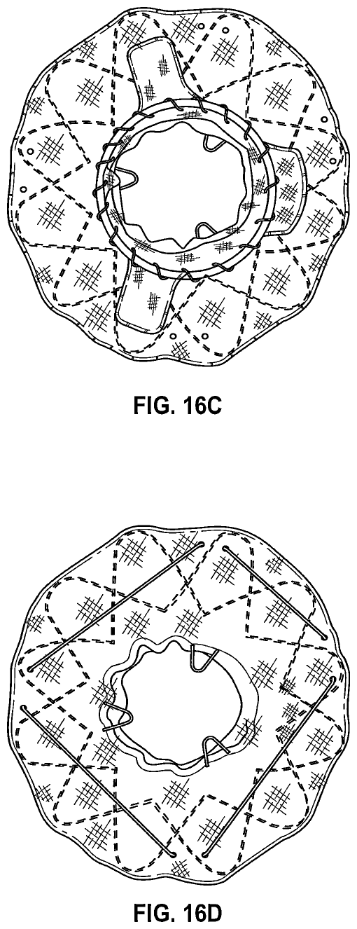

FIGS. 16A and 16B generally illustrate embodiments of the valve replacement as disclosed herein. FIG. 16A shows a top view of a valve replacement, and FIG. 16B shows a bottom view of the valve replacement. As shown in FIGS. 16A and 16B, the inflow end of the valve replacement (shown in FIG. 16A, while an outflow end is the end opposite the inflow end) may comprise anchor-retracting chords coming through the flow portion and anchoring to the underside of a flange. These sutures permit control of the anchors by pulling and releasing the chords. Alternatively, the sutures may be releasably attached to a delivery system to provide similar manipulation of the anchors. FIG. 16B further discloses the chords attached to the anchors. FIGS. 16C-16D show attachment configurations for a collapsible flange.

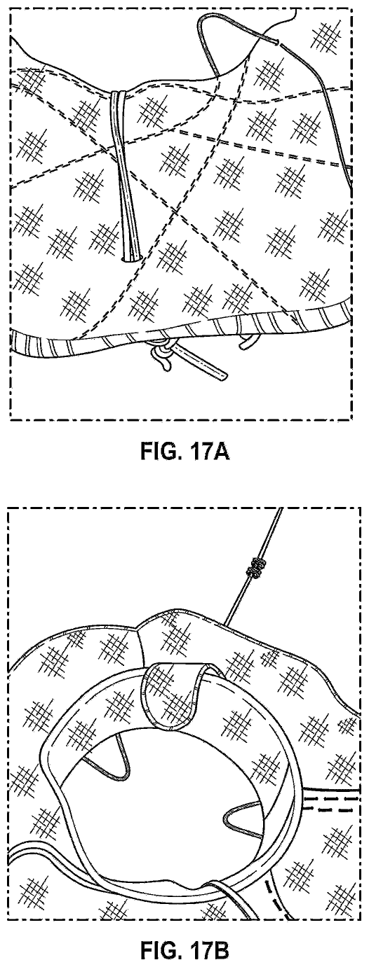

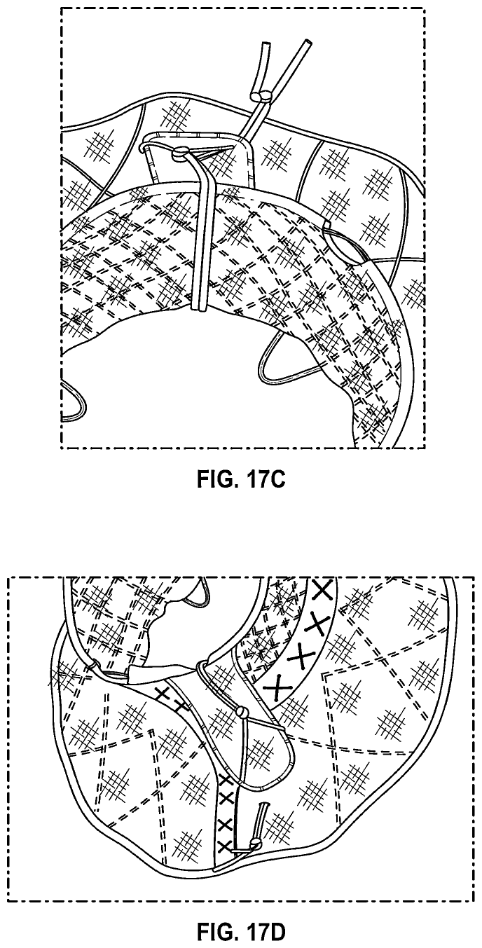

FIGS. 17A-17D generally illustrate embodiments of the valve replacement as disclosed herein. FIGS. 17A-17D show the attachment configurations for collapsible anchors and clips, and further disclose a close-up view of a suture pattern that is used to collapse and control the anchors from all angles of the valve replacement. In these embodiments, a delivery component, such as one comprising one or more suture lines, is connected on a first end to the engagement attachment, wherein the one or more suture lines connects on a second end to a controlling mechanism.

### Valve Assembly

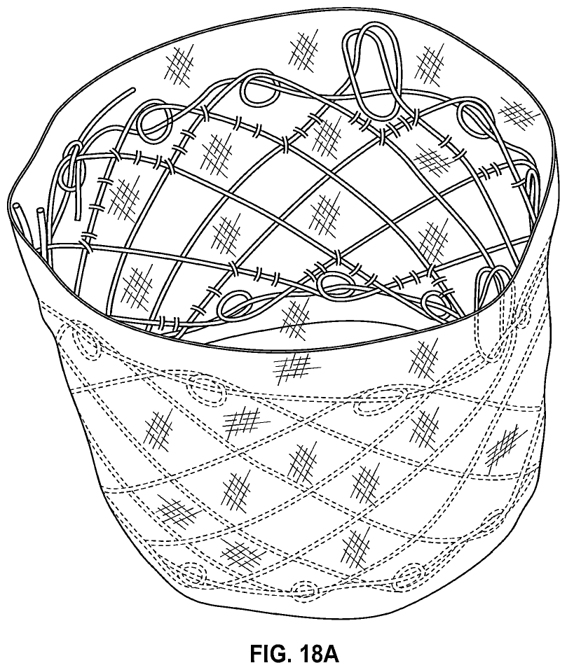

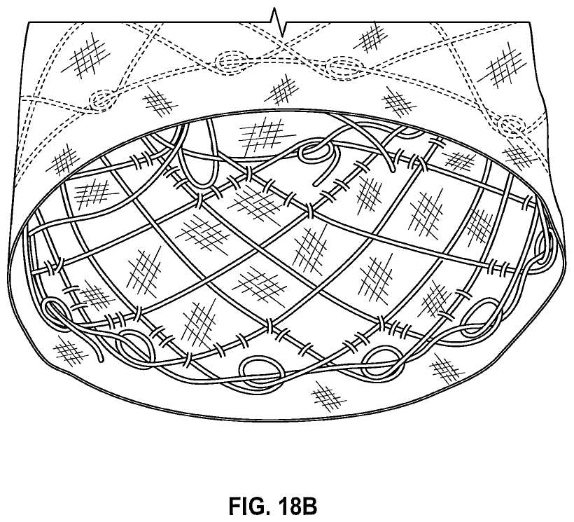

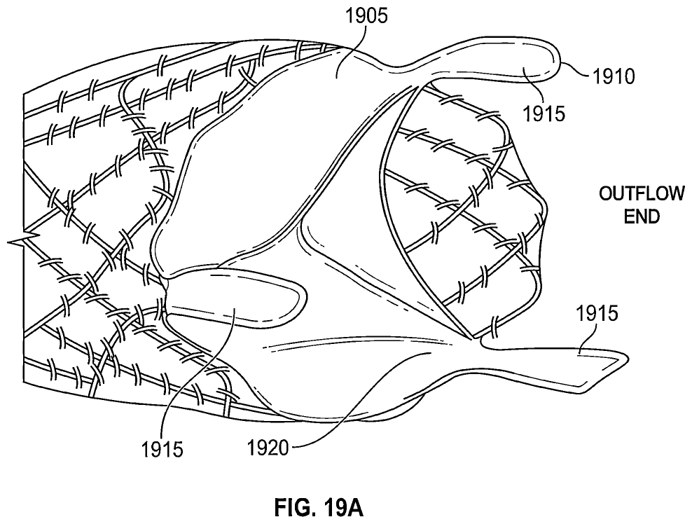

FIGS. 18A and 18B generally illustrate embodiments of the valve replacement as disclosed herein. As shown in FIGS. 18A and 18B, an embodiment of the valve replacement may be fabricated using a constraint to hold an adapter frame at a specific dimension while attaching material to influence device performance. A fabrication technique is disclosed, which acts to influence the disposition of a braided wire frame—removing the inherent freedom of movement and unpredictability that is present between relative members of the frame structure when in a load-free state. This technique involves restraining the radial expansion of the frame with a constraint, such as feeding some number of sutures through or around the structure to hold it at a specific dimension other than its unrestrained, “free” dimension. In subsequent fabrication steps, the structure is incorporated into an assembly that adopts this new configuration and considers this to be the final dimension. When the constraints are removed from the braided frame, this braided frame tries to recover to its original “free” dimension—applying additional radial force to the surrounding structure while being constrained to the desired dimension.

The degree of radial force transmitted to the fabric material from the frame can be adjusted as required to achieve the optimal combination or performance properties. In particular, the strain energy density of the structure can be more uniform. A greater stiffness is achieved (resulting in a better seal) with less material, resulting in a more low-profile structure. The suture finally provides a biasing of the structure toward a desirable diameter and height for the valve structure.

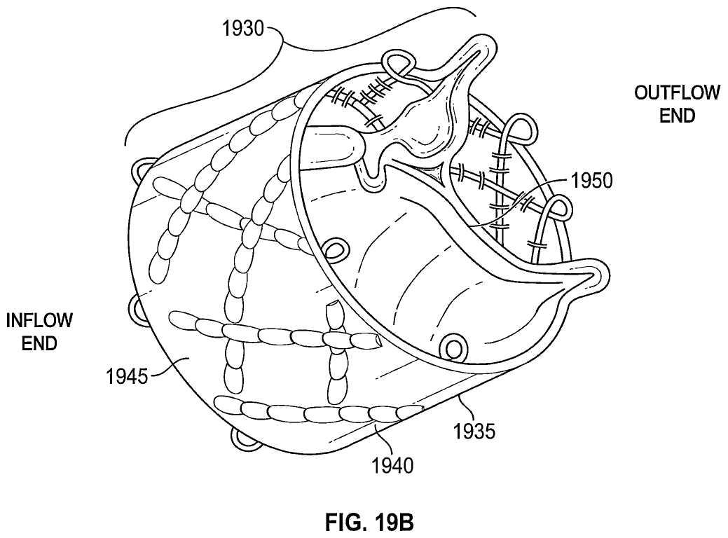

FIGS. 19A and 19B generally illustrate embodiments of the valve replacement as disclosed herein. FIG. 19A generally illustrates an embodiment of the valve assembly wherein leaflets 1905 are assembled to each other and/or to the frame by sewing. The leaflets are joined at commissure seams 1910 and then sewn, welded, or otherwise attached to the commissure posts 1915 (located at an outflow end, while the inflow end is opposite the outflow end) as well as to other points on the frame, such as wires or wire intersections, or to materials attached to the frame, for example the leaflets being attached to a cuff, which cuff is then attached to the frame. Once the assembly is complete, the leaflets work in concert to close on the outflow (distal side when being implanted into a mitral valve) when fluid pressure is increased distally, so that the leaflets close or coapt in a Y-pattern 1920.

As shown in FIG. 19B, a valve assembly 1930 comprises a braided frame having a cuff covering 1935 on the outside. This cuff over the complete outer frame may serve as an extended sealing zone. A belly stitch 1940 may be sewn to the frame whereas a bellows stitch 1945 is not sewn to the frame. In this embodiment, the distal leaflet ends 1950 are shown coapting so as to close the valve in a loose Y-shape. In some embodiments, the valve coapt area may comprise some “looseness” so as to ensure sufficient and effective contact among all three leaflets and ensure complete closing of the valve. Leaflets may be constructed of tissue such as porcine pericardium or other materials known to the art. In some cases, valves or parts of valves excised from animals may be sewn into the disclosed frame structure.

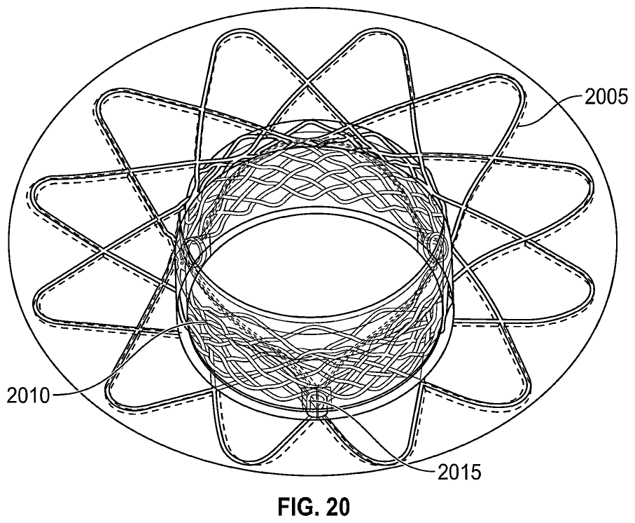

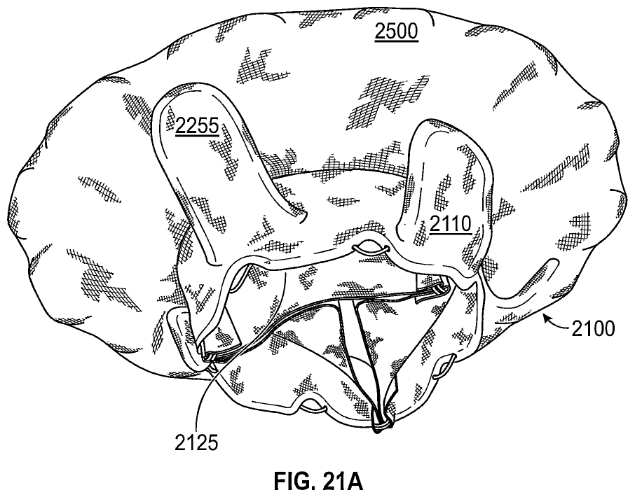

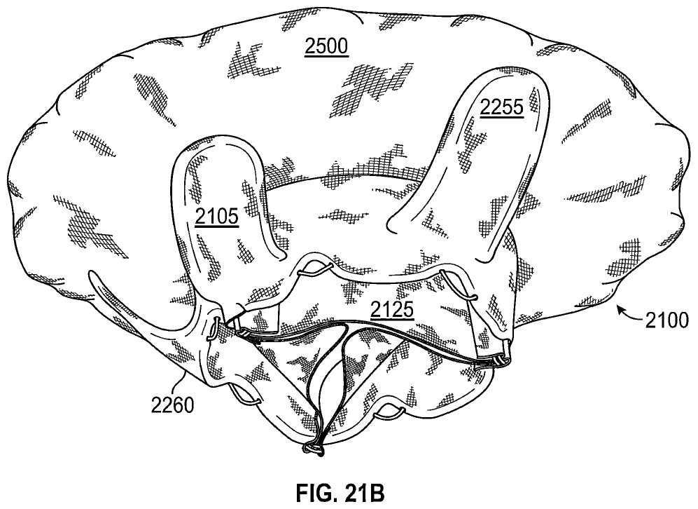

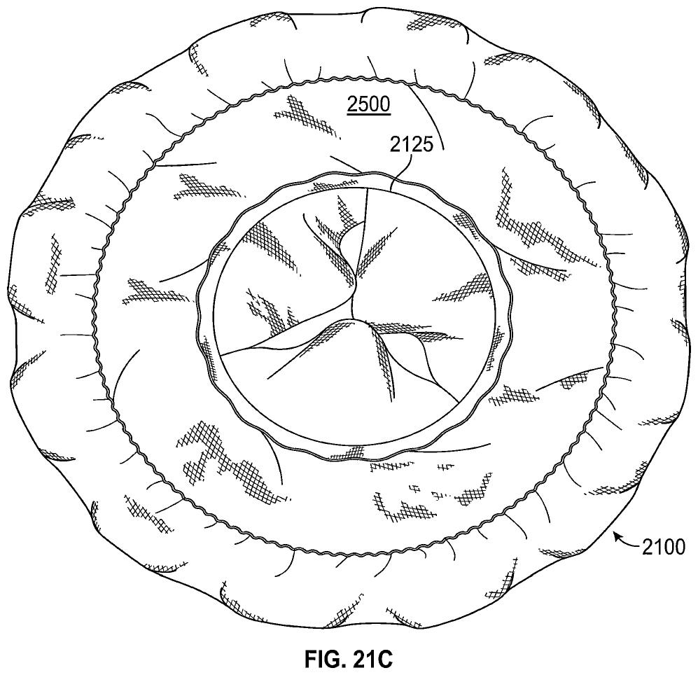

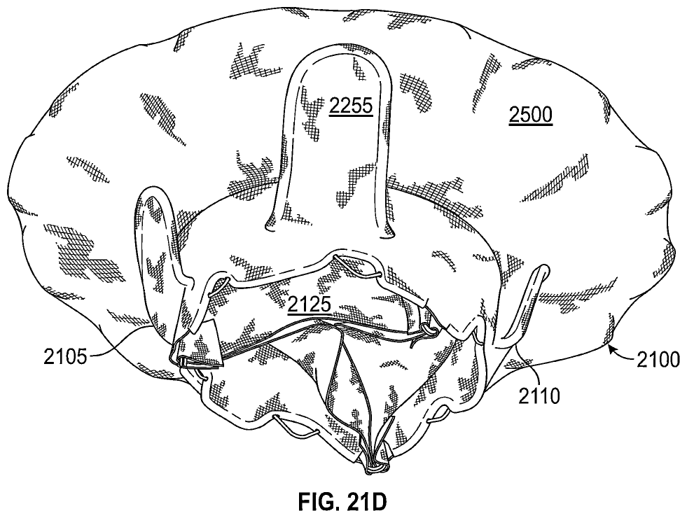

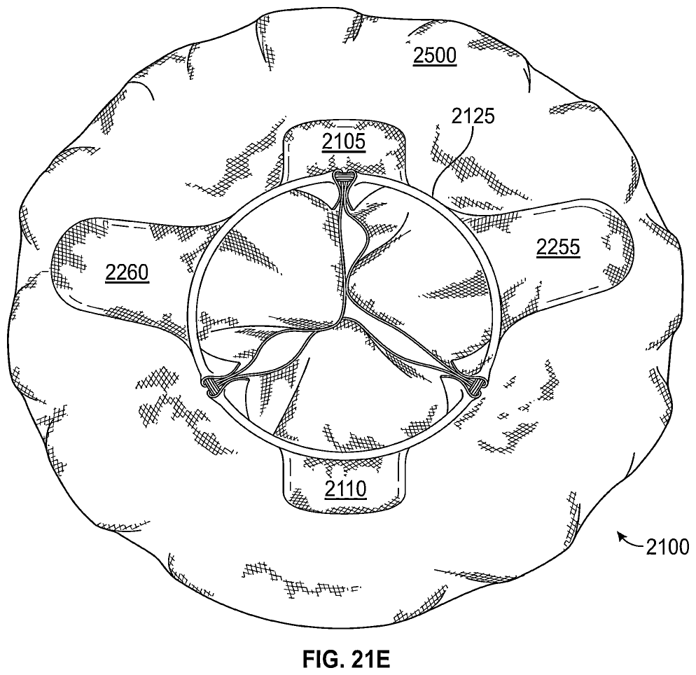

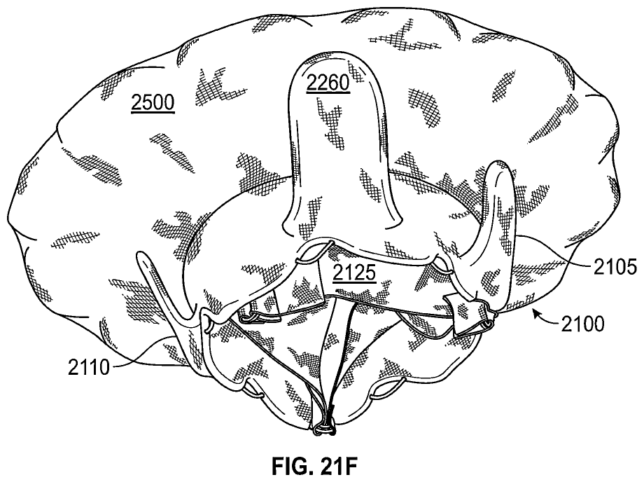

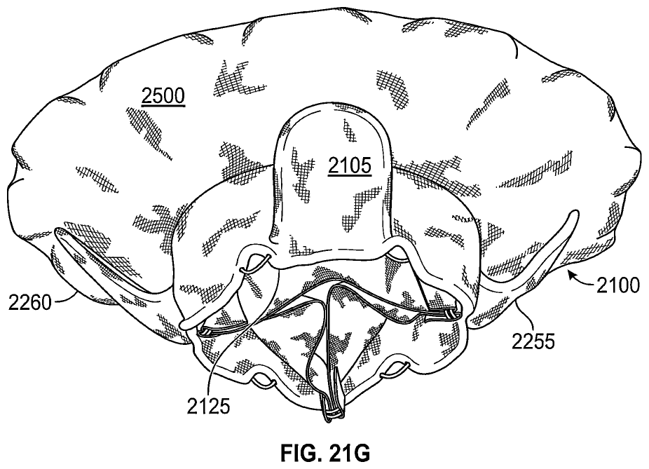

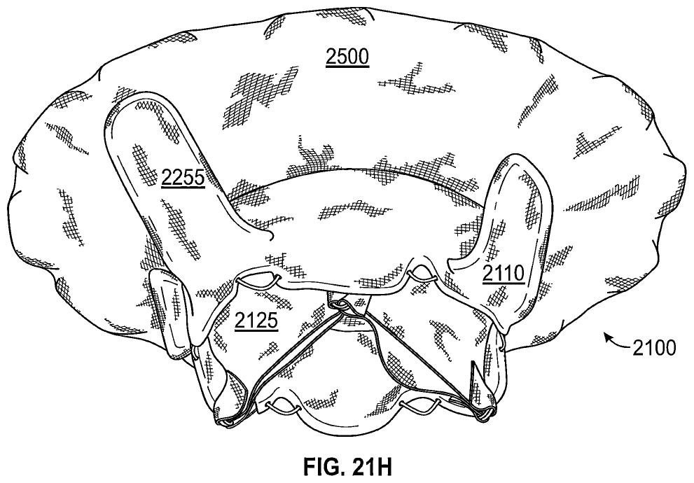

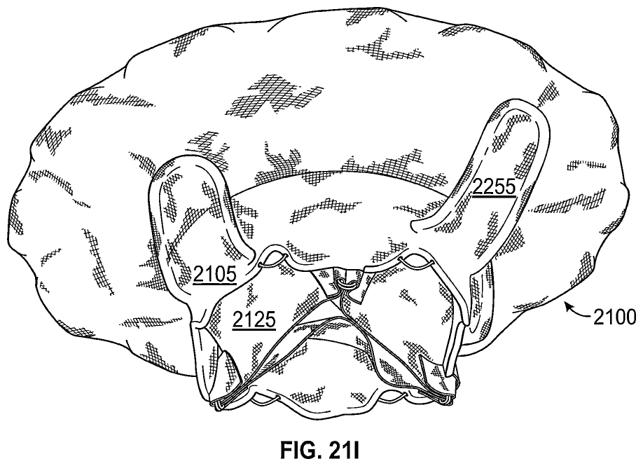

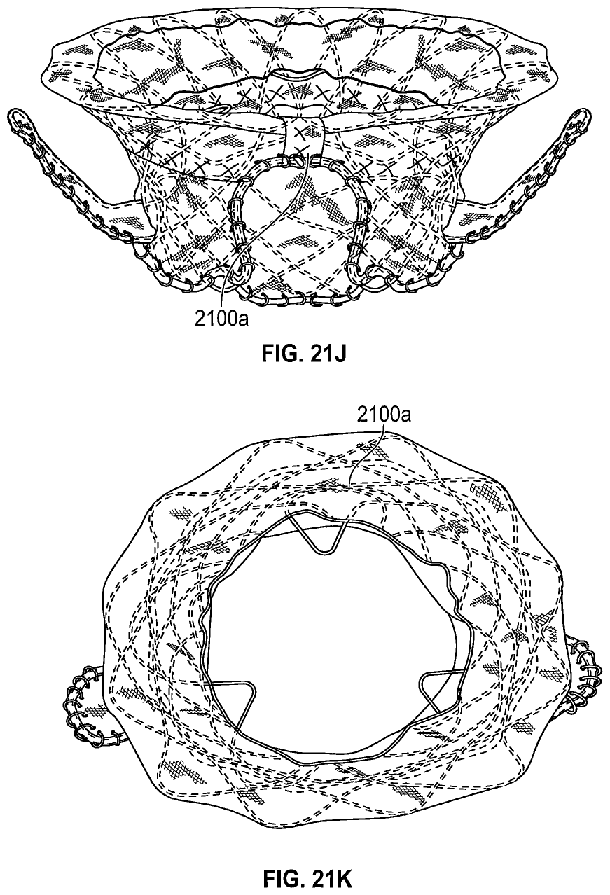

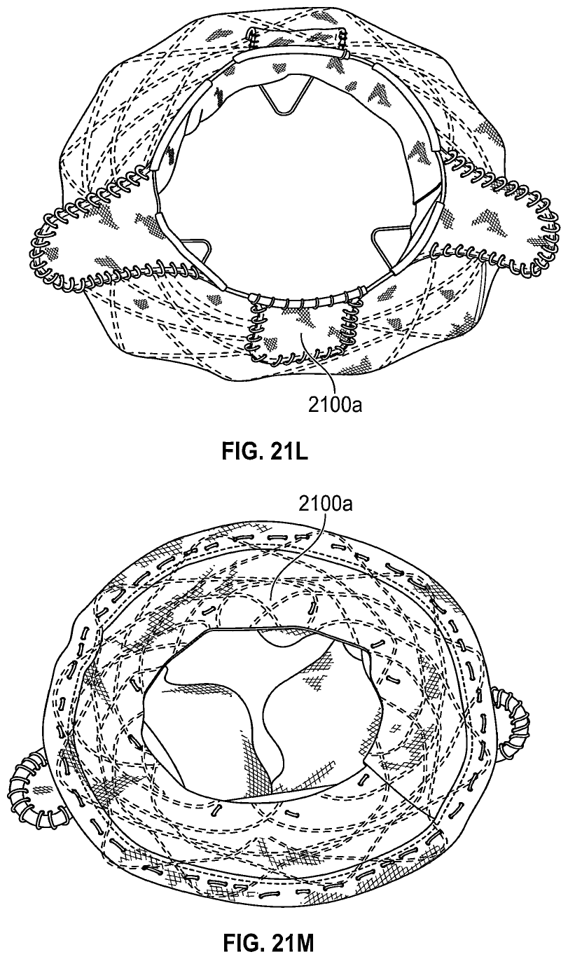

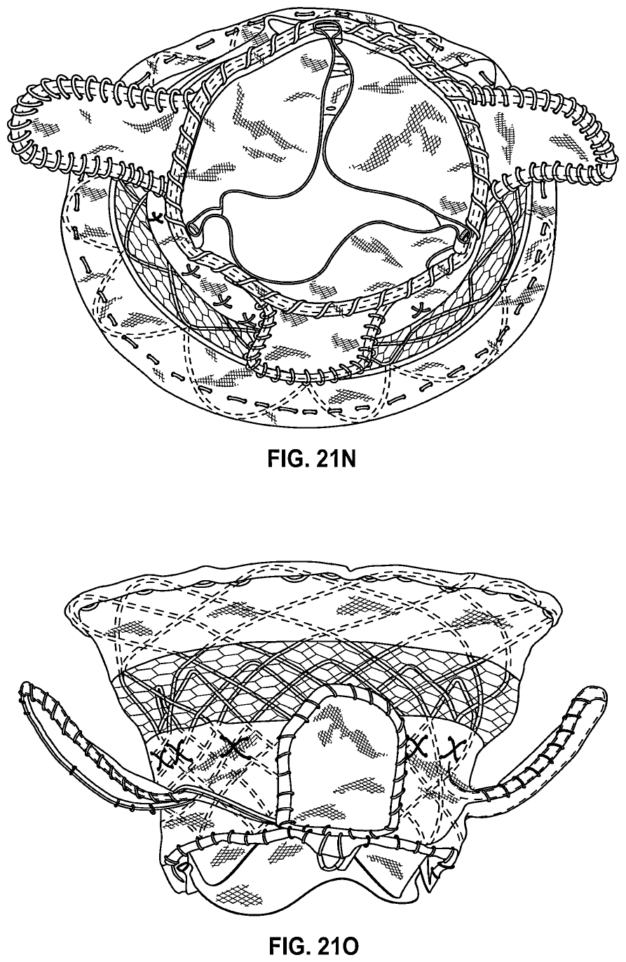

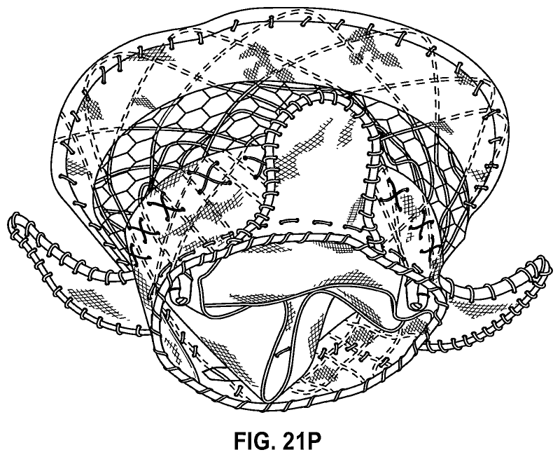

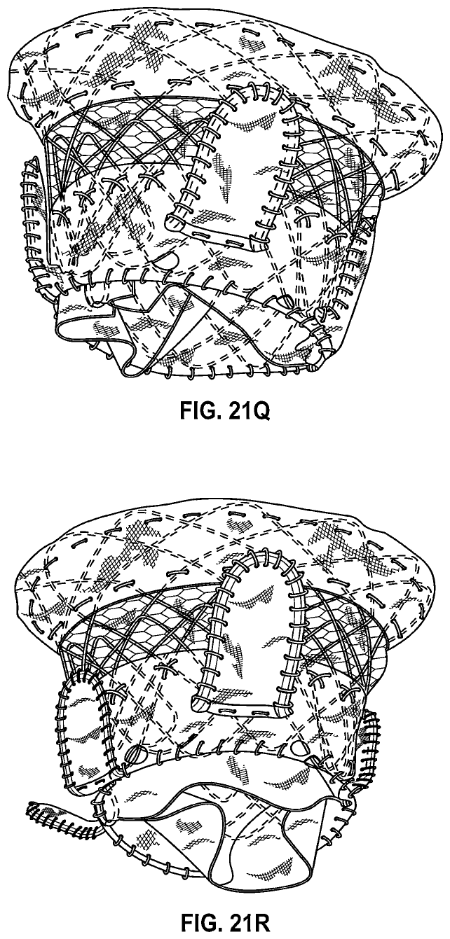

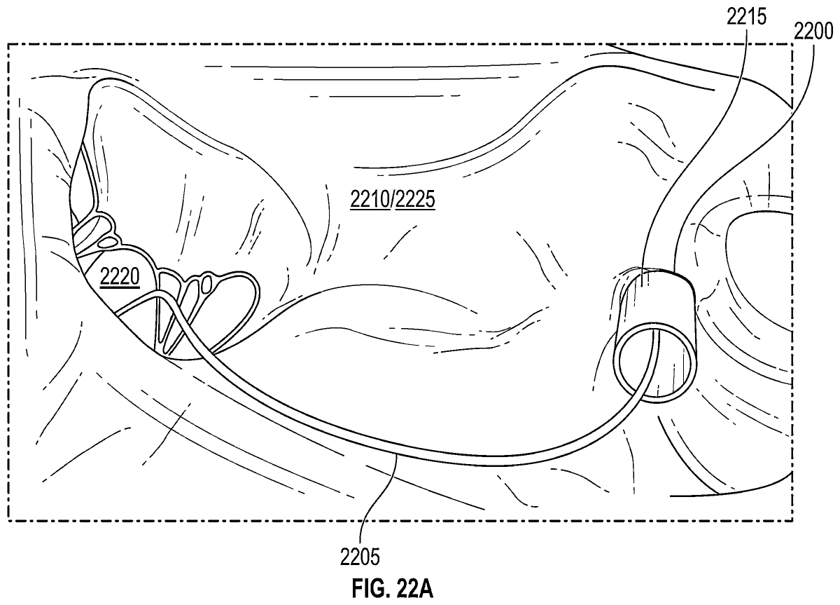

FIG. 20 generally illustrates an embodiment of the valve replacement as disclosed herein. FIG. 20 shows a bottom perspective view of the one-piece system comprising a braided wire frame making up a flange 2005, an adapter body 2010, commissure posts 2015 compatible with leaflets. In some embodiments, commissure posts 2015 may extend out from the valve replacement and the braided wire frame thereof, in some examples, the commissure posts 2015 may be configured to connect to the non-native leaflets (not shown in FIG. 20, but shown without reference numbers in FIGS. 21A-R). For example, the non-native leaflets may be cut and folded and stitched to the commissure posts 2015 and may in some embodiment extend or jut out somewhat beyond the plane of the valve replacement and/or the braided wire frame thereof.

FIGS. 21A-I generally illustrate an embodiment of the valve replacement 2100 as disclosed herein. The valve replacement embodiment 2100 of FIGS. 21A-I may show a system embodiment of and be used to perform method embodiments relating to TMVR.

Some solutions to TMVR described herein may utilize the valve replacement embodiment 2100 and involve several types of securement. For example, the valve replacement embodiment 2100 may feature one or more types of securement, and in some embodiments more than two securement mechanisms For examples, some valve replacement 2100 embodiments may utilize four-point securement. In some embodiments, such multipoint securement may be configured to distribute an implant's workload across an entire device by utilizing a combination of securement mechanisms. In contrast, some prior art devices may only utilize (or primarily use) the radial force (as described in more detail below)—essentially relying on “brut radial force”—for their positioning, securement, and scaling.

For example, one type is supra-annular securement. This may be accomplished in some embodiments through a flange being placed, clamped, or cinched onto the annulus or to ledges (e.g., mitral ledges) above the annulus. In embodiments, the flange may prevent migration into the ventricle and may be configured and/or designed to eliminate paravalvular leaks. Another type is sub-annular securement. Utilizing anchor features, which may be struts 2255, 2260 in some embodiments (and as described in more detail below), such securement may provide stability in the medial and lateral positions directly under the anatomy of the native heart valve in the sub-annular region. For example, in embodiments, the sub-annular anchors perform as struts or braces that permit slight movements of the valve replacement with the natural helical movement of the native heart, but constrains movement of the valve replacement, for example in medial and lateral positions or directions or in anterior and posterior directions, so that the valve replacement does not migrate into the atrium of the native heart or have paravalvular leaks.

Such sub-annular securement may also involve, or relate to, other anchor features, which may be clips for securing to native tissue, including leaflet securement. Some embodiments may thus utilize leaflet clips 2105, 2110. In some embodiments, the struts 2255, 2260 may be more elongated than the clips 2105, 2110, and jut outward farther from the device than the clips, with strut tips designed and/or configured to press against and jut into native anatomy (e.g., the trigone area). On the other hand, in some embodiments the clips might have a more curved shape, e.g., curving upward and bending inward toward the native leaflets, or otherwise similarly configured hold leaflets in place.

In some embodiments, the anchor features for securing native leaflets may be hangers, which may hang onto or capture the leaflets, rather than clips 2105, 2110 to clip onto the native leaflets. Such securement may prevent migration into the atrium and control movement of native leaflets. In some embodiments, and as described elsewhere herein, the aforementioned leaflet clips 2105, 2110 may be deployed before deployment of the struts 2255, 2260. Such sub-annular securement, for example, may be sufficiently restrictive enough to prevent the device embodiment 2100 from migrating into mitral area or creating leaks, yet loose enough to move with natural movement of the heart. Such movement, and in accordance with other aspects of this disclosure, may be facilitated by overlapping wire structure without many rigid fixed points. In embodiments, native leaflets may be captured by leaflet anchors or engagement attachment (e.g., clips), which in embodiments may hang on the native leaflets (rather than pinch the native leaflets) to prevent migration towards the native atrium, and anchor struts, which in embodiments transfer compressive loads to the native annulus or native anatomy near the native annulus in the inter-commissural zones below the annulus in the native ventricle area, to prevent migration into the native atrium.

With regard to leaflet securement, in some examples, different points in the native tissues may be associated with securement features. For instance, in some examples, the valve replacement embodiment 2100 may integrate four points for anchoring, scaling, and fixation. In the embodiment shown, two general points or regions of native leaflet tissue may be associated with two leaflet anchors or engagement attachment (e.g., clips) 2105, 2110 and two general points or regions of native tissue may be associated with two anchors or struts 2115, 2120. These multiple points of securement may provide stability while preserving the LVOT, preventing paravalvular leaks, and trauma to tissue associated with the native heart wall.

Such multi-point securement using the valve replacement embodiment 2100 may result in a highly flexible and conformable valving system. The system may encourage structural stability by preserving central flow through the valve.

Another type of securement is selective radial force securement. In some embodiments, directional radial force securement may be controlled through a receiver or adapter, which may assist in preventing migration while preserving the LVOT. Such radial force, in some examples, may be generated by oversizing a valve frame 2125 (e.g., a wrapped or braided nitinol wire frame around a mandrel) with respect to the annulus hole, pushing against the annulus walls. In some embodiments, the valve frame 2125 may be shape-set to about a 10% oversize in relation to the annulus hole, while permitting some limited movement. Such radial force securement may also prevent migration into the atrium and ventricle while preserving LVOT.

In addition, as explained elsewhere, the natural helical structures of features associated with valve frame 2125 embodiment may be based on the more holistic understanding of the nature of the heart and its motions. For example, such features may be configured to mimic the movement of a healthy heart using natural helical structures (as described above), by contracting and twisting with each beat of the heart.

Such features may include braided wire designs (as explained and shown elsewhere in more detail), which in some embodiments may offer increased flexibility and conformability. In addition to such braided wire design embodiments adapting to and moving with the heart, they may be configured to (by, e.g., integrating diverse and various wire thicknesses and braiding designs) forgive anatomical anomalies, and conform with various densities and characteristics (i.e., radial force and expansion) of the heart's anatomy.

Further, braided wire design embodiments may leverage nitinol strength through geometry and a unique braided wire architecture. Such architecture in some embodiments may purposely omit fixed nodes at crossing points of wires and permit the braided wires to move across one another in a controlled fashion. Such features may assist the replacement valve in moving with the natural helical movement of the heart. Some braided wire design embodiments may also facilitate placement in the native heart, maximize seal in the human anatomy, and prevent unwanted migration with an integrated and optimized novel securement system. Thus, such features may be designed based on recognition that the mitral valve is more than simply a structure to be “stented.”

In contrast, some earlier valve frame designs by others (e.g., centering on laser cut nitinol on a lattice) may not feature such helical architecture or otherwise be configured for allowing similar dynamic movement. Rather, such prior art designs may often be limited to fixed nodes across a lattice design, or may not permit use of various wire thicknesses, thereby impeding the requisite flexibility and conformability for the human heart.

These different ways of securement (as mentioned above) may assist in distributing the valve replacement embodiment's 2100 (e.g., the implant's) workload across the entire device 2100, and may provide for maximum stability while preserving the LVOT and preventing paravalvular leaks and trauma to the native heart wall. In addition, such multiple ways of securement and multi-point anchoring systems may enable methods (as also described herein) allowing a simpler and more secure approach to Transcatheter Mitral valve replacement and resulting in a safer overall procedure for patients.

FIGS. 21J-M generally illustrate an embodiment of the valve replacement 2100a as disclosed herein. Similar to the valve replacement embodiment 2100 shown in FIGS. 21A-I the valve replacement 2100a may have two leaflet clips (such as leaflet clips 2105, 2110) and two anchors (such as anchors 2115, 2120) for securement to native heart tissue. Unlike the flange embodiment shown in FIGS. 21A-I, which may feature more of a flatter surface and more of a right angle at its transition point or zone, flange embodiments shown in FIGS. 21J-S may have more of a curved or funnel shape, where the transition point or zone corresponding to the annulus may also continue further down into mitral valve, where it may flex and push outward to fill in gaps between the valve surfaces and the native anatomy. In some examples valve replacement embodiments, the transition zone may be between the flat flange portion (which may be configured to rest on top of annulus in the atrium) together with and including the funnel-like contoured ring of the flange and the adapter portion that radially pushes out into the annulus. Such a contoured transition portion may assist in promoting central vortex flow and provides improved scaling and anchoring. The funnel-like portion may have shape-set braided wire and material covering that is contoured and configured to fill in the gaps between the native anatomy and the transition zone between the atrium and annulus heading towards the ventricle area.

FIGS. 21N-R generally illustrate an embodiment of a valve replacement 2100b as disclosed herein. Similar to the valve replacement embodiment 2100 shown in FIGS. 21A-J, and to valve replacement 2100a shown in FIGS. 21K-M, valve replacement 2100b may also have two leaflet clips (such as leaflet clips 2105, 2110) and two anchors (such as anchors 2115, 2120), for securement to native heart tissue. However, valve replacement 2100b may have a different layer of material than valve replacement 2100a, with different design and covering less than all surface area of the valve replacement 2100b.

### Delivery Method Embodiments

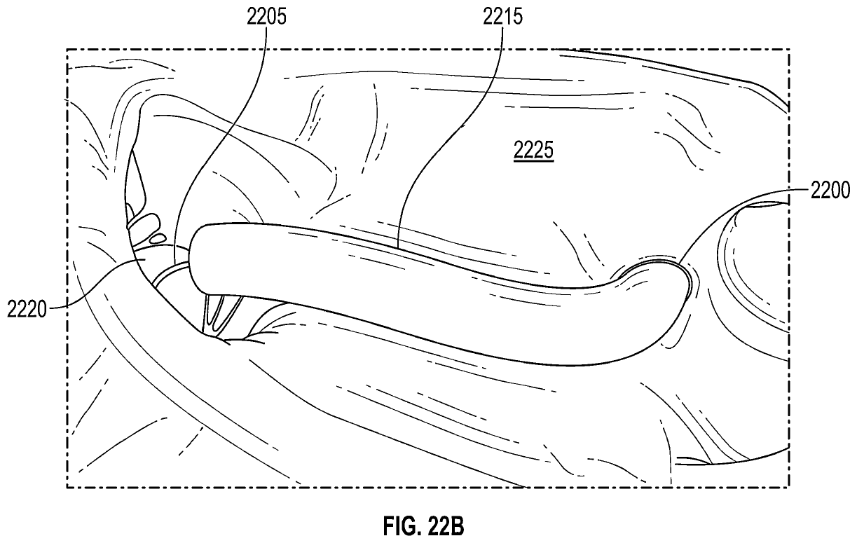

FIGS. 22A-26B may show a method and/or system of delivering a valve replacement, incorporating valve replacement embodiments such as valve replacement embodiment 2100. In some examples, the method and/or system may involve placing the two-piece adapter and MLS together as one piece and loading them together into a delivery system, in accordance with aspects of this disclosure, and as described below. Some method embodiments may apply as well to a one-piece device consisting of both an adapter and an MLS as a single device.

As an initial step of a method embodiment described herein, a sheathed valve/implant 2215 may be positioned over a mitral valve through a transeptal procedure. For example, FIG. 22A shows a transeptal puncture 2200 (in e.g., the transfemoral area) through which a guidewire 2205 may enter the left atrium 2210. The guidewire 2205 may be used by the method and/or system to assist in delivering a valve replacement, such as valve replacement embodiment 2100.

The method and/or system described herein may allow a broad plane of movement, allowing flection/deflection of the guidewire (and/or other delivery system components) in several directions, such as in the medial and lateral and anterior and posterior directions. Utilizing such directionality, the guidewire 2205 may also enter the mitral valve 2220.

FIG. 22B shows next a sheathed valve/implant 2215 entering the atrium 2210 along the guidewire 2205.

Following the guidewire 2205, the sheathed valve/implant 2215 may be centered over the mitral valve 2220, in order to enter the ventricle 2225 over the wire 2205. Some embodiments of the delivery method described herein may also include verifying that there is clearance over the mitral valve 2220 and that there is proper flection for movement before further advancing downward. In some examples, the method may include tracking the sheathed valve/implant 2215 as it moves down through the native heart structure.

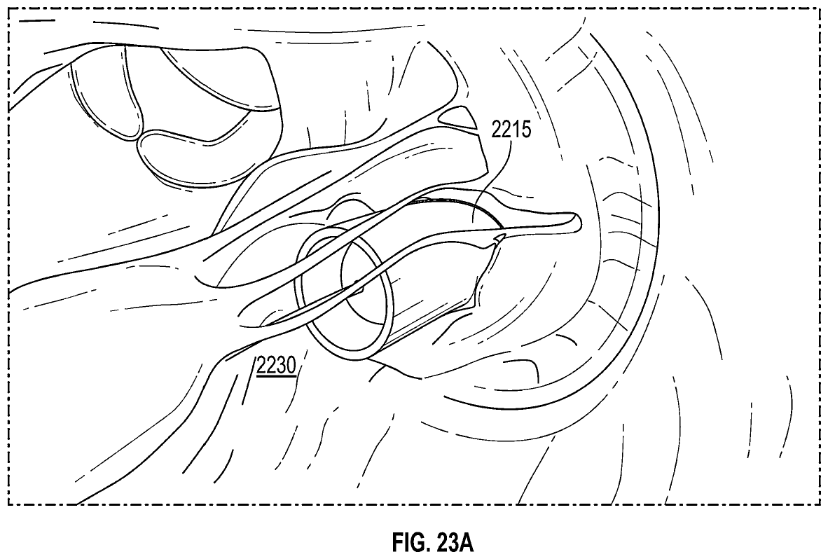

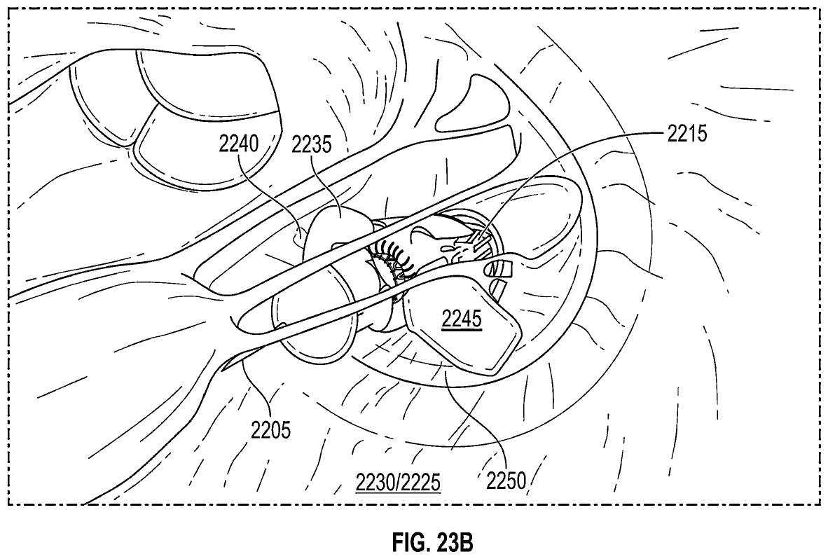

As shown in FIG. 23A, the sheathed valve/implant 2215 may then be moved down into the ventricle 2230 from the septal puncture. Some method embodiments may include a step of testing a path or runway before moving further into the ventricle 2230, retracting, and then returning to move further down into the ventricle 2230, to ensure that the path is not obstructed and that a correct path has been chosen.

FIG. 23B shows, from the ventricle perspective, the sheathed valve/implant 2215 moving further down into the ventricle 2225/2230 for deployment of sub-annular anchors. In some examples, the anterior clip 2235 may be released or extended prior to moving down in order to determine the location of the anterior clip 2235 in relation to the 360° of the device 2215. Then, as the device 2215 (including the implant) is advanced into the ventricle, and based on the determined position of the anterior clip 2235, the anterior clip 2235 may be oriented or lined up with the location of the anterior leaflet 2240.

In some embodiments, once in the ventricle 2225, the anterior clip 2235 (which may also be referred to as the A2 clip 2235) may be unsheathed so that it is aligned with the anterior leaflet 2240. Then, after finishing entering the ventricle, the anterior clip 2235 may gently slide across the surface of the anterior leaflet 2240 into a predetermined position (e.g., the A2 region) for securing the anterior leaflet 2240. In some embodiments, the position may include the anterior leaflet 2240 being behind the anterior clip 2235. The anterior clip 2235 may be used to envelope the anterior leaflet 2240, and in some embodiments, the anterior leaflet 2240 may also be secured, which may include the anterior clip 2235 grabbing the anterior leaflet 2240 and/or a particular area thereof (e.g., the A2 region), to provide sub-annular securement and prevent migration of the valve replacement into the atrium.

Next, once the anterior leaflet 2240 is secured within the anterior clip 2235, the posterior clip 2245 which may be referred to as the P2 clip 2245) may be unsheathed or released so that it is proximate to the posterior leaflet 2250. In some embodiments, the posterior leaflet 2250 may be behind the posterior clip 2245. In some embodiments, the posterior clip 2245 may be used to envelope the posterior leaflet 2250, and in some embodiments, the posterior leaflet 2250 may also be secured, which may include the posterior clip 2245 grabbing the posterior leaflet 2250 and/or a particular area thereof (e.g., the P2 region), to provide sub-annular securement and prevent migration of the valve replacement into the atrium.

Securing both the anterior and posterior leaflets 2240, 2250 may prevent those native leaflets 2240, 2250 from interfering with the functioning of new leaflets 2265, which may be artificial or bovine leaflets. Once the clips 2235, 2245 are both in proper positions (in the inter-commissural or commissure-to-commissure space) and secured, the device 2215 may be slightly retracted towards the atrium 2230. In embodiments, the clips 2235, 2245 provide securement by a distal end of the clips (the free end opposite where it is attached to the valve replacement) pressing up against an underside of the annulus to prevent dislodgement or migration of the valve replacement.

In some embodiments, the clips 2235, 2245 may include two vertical, smaller loop structures configured to open and close and connect with the leaflets 2240, 2250, thereby enveloping (but not necessarily “pinching”) the leaflets 2240, 2250. In this manner, for example, the clips 2235, 2245 may envelope the anterior leaflet 2240 (which may be closer to aortic valve 2405) and the posterior leaflet 2250, and/or parts thereof. In some embodiments, an anterior clip 2235 may be an A2 clip for securing the A2 anterior region of the anterior leaflet 2240, while the posterior clip 2245 may be a P2 clip for securing the posterior region or a particular part thereof. In some embodiments, enveloping native leaflets 2240, 2250 at these regions may provide a particular desirable form of securement (but other forms are contemplated).

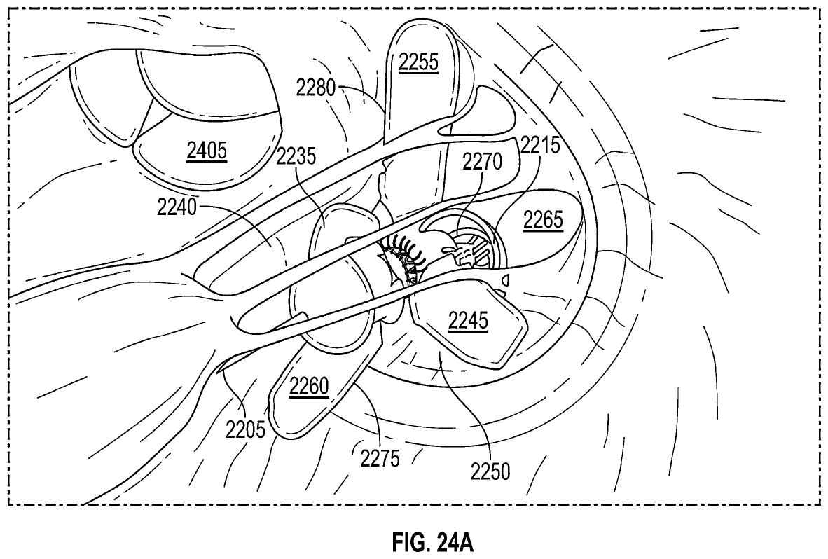

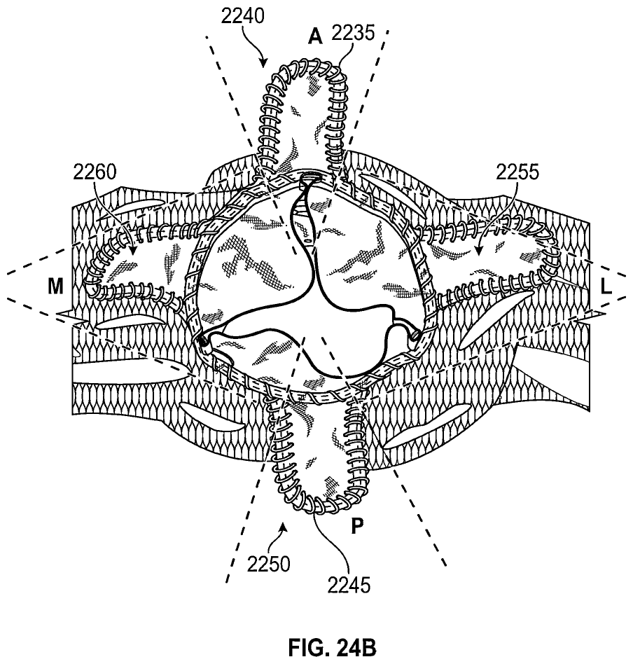

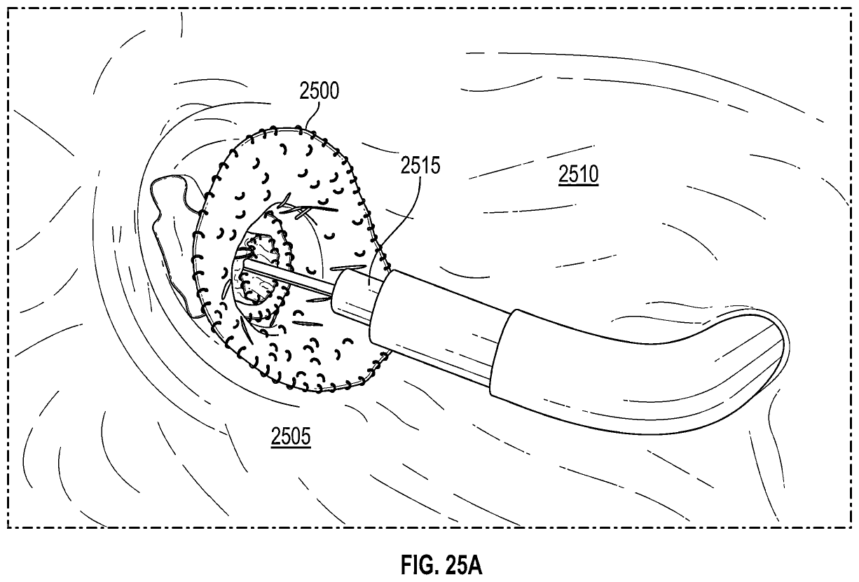

FIG. 24A generally illustrates an embodiment of a valve replacement as disclosed herein. FIG. 24B also generally illustrates an embodiment of a valve replacement as disclosed herein. Relatedly, embodiments of the method described herein may further include releasing medial and lateral strut anchors 2255, 2260, as shown in FIGS. 24A and 24B. Some method embodiments may include first pulling up the device 2215 towards the atrium, and determining or verifying that the strut anchors 2255, 2260 are below the annulus 2265 and not in the atrium 2230. Some ways of so determining include, e.g., using echo technology, and using 3D and 2D imaging to identify locations of the tips of the anchors, and/or to otherwise identify locations of the anchors (and to ledges, which are further explained below).

In some embodiments, the clips 2235, 2245 may be released or extended based on some trigger mechanism to be controlled by an operator, which may involve, e.g., pulling a type of (e.g., a first) string.

FIG. 24A shows unsheathed medial anchor 2260 and lateral anchor 2255. In some embodiments, the extended or released medial 2260 and lateral anchors 2255 may anchor to, or rest in, the trigone area (which may be proximal to the anterior region of the sub-annular portion of the native heart) of the native tissues, and potentially in between chordae. In some embodiments, there may be desired target areas of native tissue for the anchors. For example, the medial anchor 2260 may rest on/in the medial area 2275 of native heard tissue while the lateral anchor 2255 may rest on/in the lateral area 2280 of native heart tissue.

In some embodiments, the distance or diameter from the medial and lateral anchors 2255, 2260 to the anterior and posterior clips 2235, 2245 may be in a range of 65° to 115°, and not necessarily 90°, and in some embodiments about 75°, from each other. In some examples, the design of the device 2215 may be such that the medial and lateral anchors 2255, 2260 and the anterior and posterior clips 2235, 2245 are spaced so that appropriately grabbing the anterior and posterior leaflets 2240, 2250 using the clips 2235, 2245 may result in the medial and lateral anchors 2260, 2255 aligning with predesignated and proper positions for anchoring. Thus, unsheathing the anchors 2255, 2260 may assist in achieving full securement on the ventricular side.

In some embodiments, operators may control not only the movement and directionality of the guidewire 2205 and device 2215, but also the releasing and unsheathing (and sequence thereof) of the clips 2235, 2245 and anchors 2255, 2260, and many aspects of delivery. It is anticipated that the relative simplicity associated with such steps, and with regard to placement, will enable a broad group of qualified operators. In some embodiments, the anchors 2255, 2260 may also be released or extended based on some trigger mechanism to be controlled by an operator, which may involve, e.g., pulling a type of (e.g., a second) string.

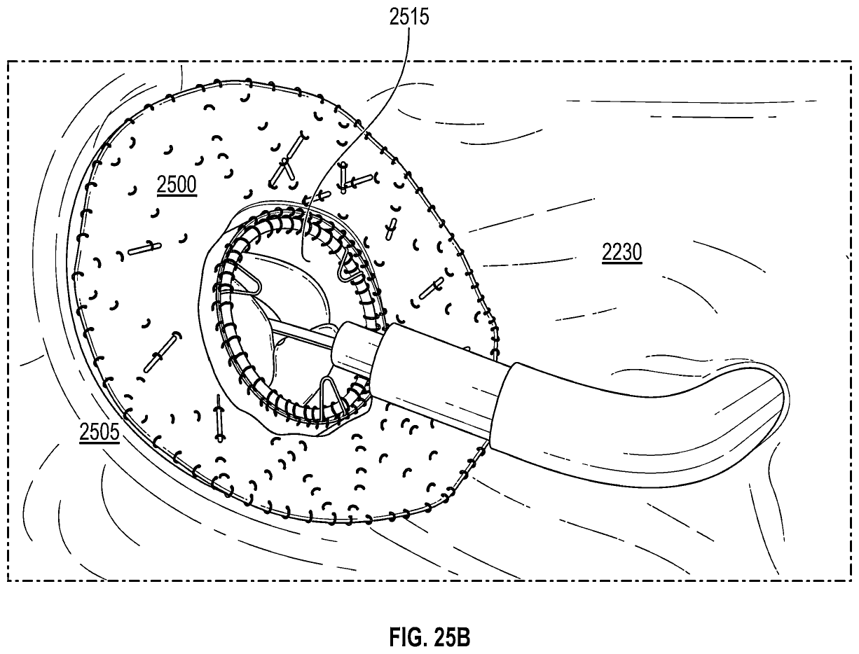

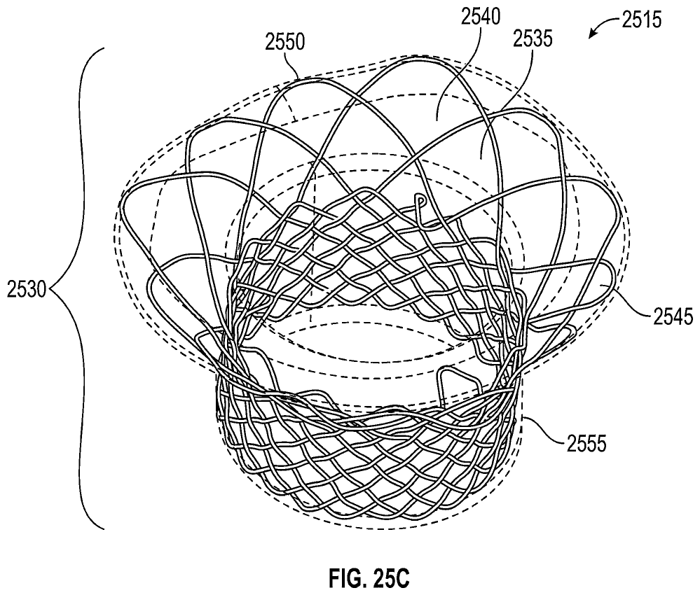

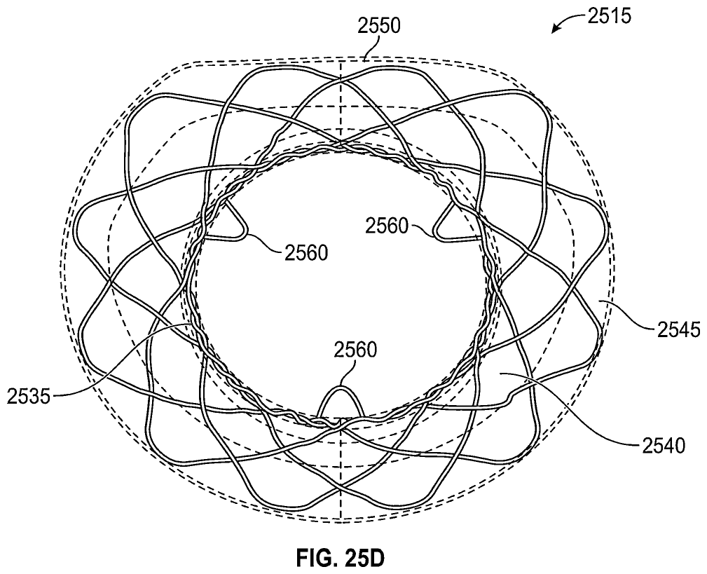

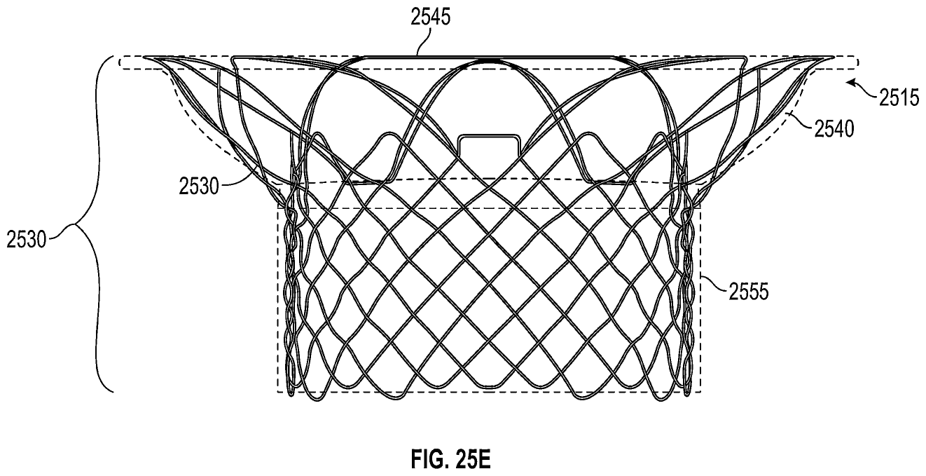

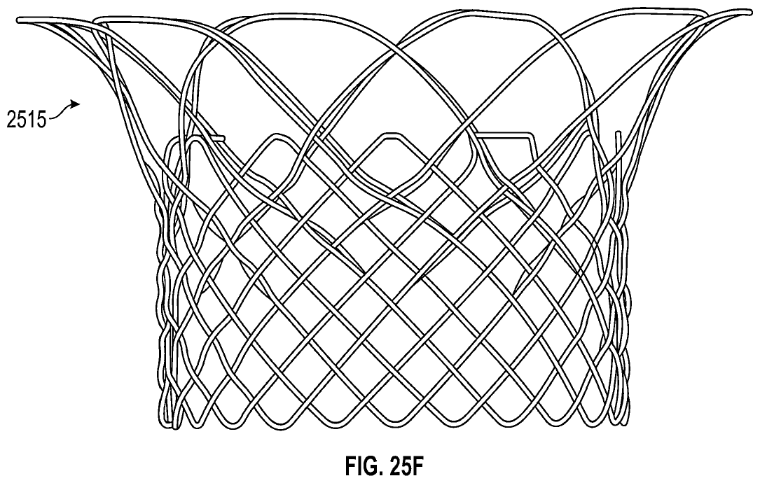

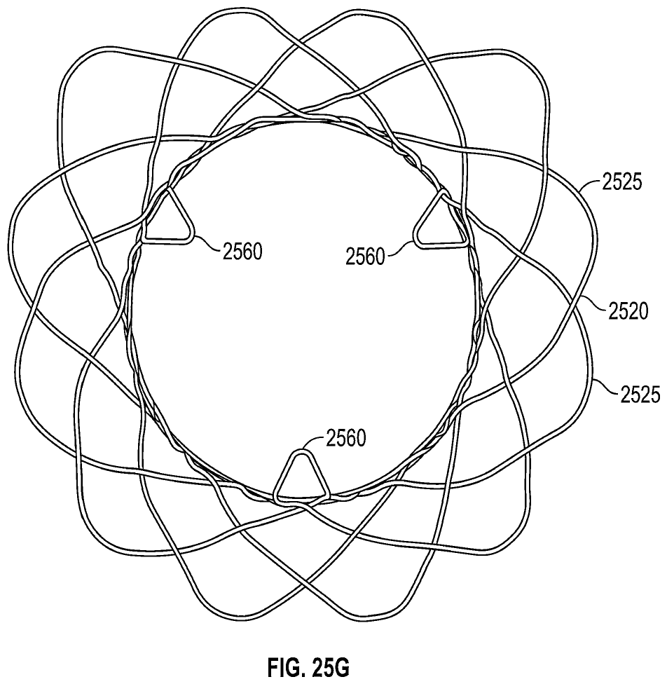

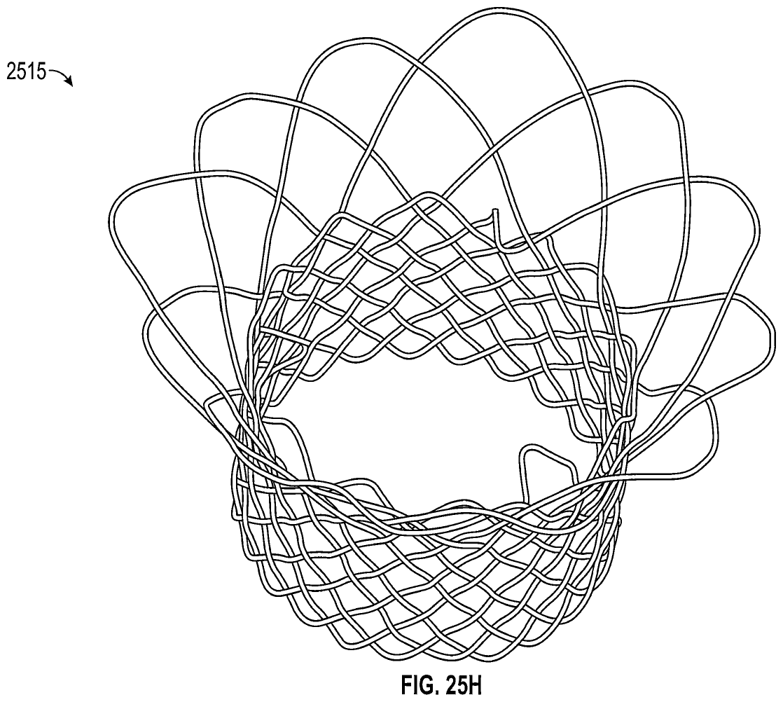

Embodiments of the delivery method described herein may also include unsheathing the flange 2500 in the annulus 2505 on the atrial side 2510, as shown in FIG. 25A (from the dorsal perspective). In some embodiments, deploying the flange 2500 may be triggered by or occur in relation to the pulling back or removing the sheathe 2515.

As shown in FIG. 25B, releasing and deploying the flange 2500 may cinch it onto the annulus 2505, and/or to ledges thereof or just above the annulus 2505. In some respects, this may assist in sandwiching or trapping the annulus 2505 between the flange 2500 and the anchors 2255, 2260 and clips 2235, 2245 (as shown, among other figures, in FIG. 24), thereby creating a greater level of securement while still permitting an acceptable range of movement. (For example, as explained above, the device may move with the contractions of the heart, not only with the squeezing of the heart, but also with the twist of the heart.) Such securement may assist in preventing the device 2215 and components thereof from inadvertently being injected or migrating into the atrium 2230/ventricle 2225 (shown in FIG. 24).

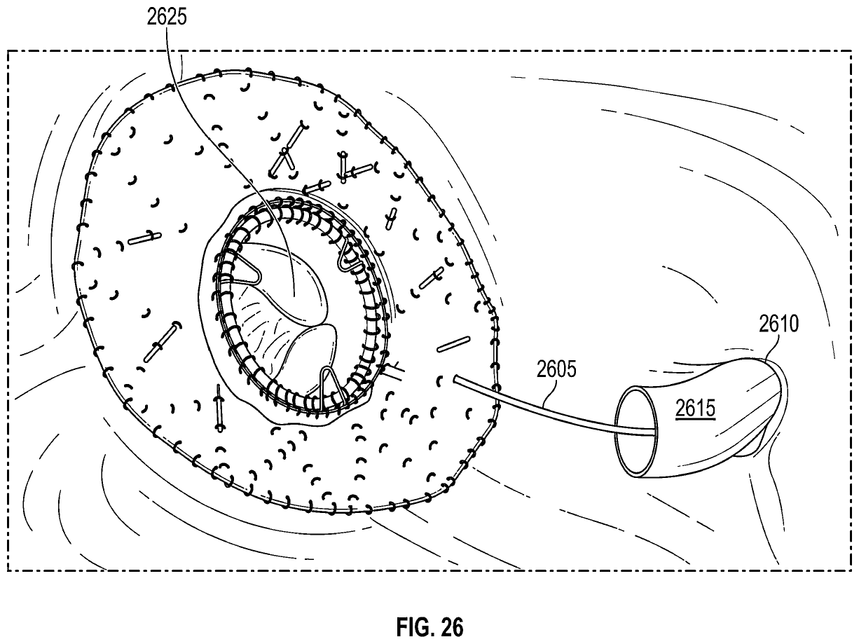

Then, once the device 2215 is in place as described above, the tube may retract and the guidewire may be removed, as shown in FIG. 26.

FIGS. 25C-25E generally illustrate embodiments of the valve replacements as disclosed herein. FIGS. 25F-25H generally illustrate an embodiment of a wire frame for valve replacements as disclosed herein.

In particular, FIGS. 25C-25E illustrate an embodiment of a flange 2515 of the valve replacement. The flange 2515 may have a contoured “funnel-like” shape fitted onto the annulus (as opposed to a “top hat”-shaped flange with a right-angle transition).

The frame of the flange 2515 may include one or more wires. For example, one embodiment may feature a flange 2515 with several (e.g., three) wires and have a braided structure, as shown in FIGS. 25F-25H, aspects of which may be similar to braided structures described elsewhere in this disclosure. In some examples the wires of the flange may form looping petals, terminating at extrados 2525. In some embodiments, the pedals may be uniform and shaped for predetermined placement corresponding to anatomy.

The angle between the intersection points 2520 of extrados 2525 may vary. In examples with a high angle (and potentially less wires), the braid may involve increased wire contact with the annulus, which may also involve a longer overall compressed length. Some such embodiments may have straighter, stiffer, and/or stronger contact with the annulus and a shorter overall compressed length.

Some flange 2515 examples and other related features may be created to have a specific desirable shape using a tooling or shape-setting process. This shape-set tooling may be derived from a geometric surface that has been carefully constructed to interface optimally with a diseased mitral annulus. In one such technique flange tips may be forced inward (toward the center of the implant). This may cause the wires to buckle in a controlled manner, potentially minimizing triangular gaps between wire extrados.

In some examples, the flange 2515 may be fabricated using a layer of material 2530 (e.g., “one-piece sock”), a contoured ring 2535 (with or without grooves), a contoured nesting ring 2540 (with or without grooves) and a top plate 2545 with a D-shaped perimeter ledge 2550. Such components may be assembled with locator pins onto a cylindrical mandrel 2555 and together control the buckling of the wires. The D-shape perimeter 2550 may be constructed to (optimally) seal the annulus, providing more coverage specifically at the native commissures and medial to the native commissures. In some embodiments, the braid itself and D-shape perimeter 2550 may provide a transition zone for anatomical features, for example having localized softness to accommodate the aorto-mitral curtain or LVOT. Further, in some embodiments, the straight side of the “D” of the D-shape perimeter 2550 may be configured to not block or press into native anatomy, and to prevent cutting off blood flow circulation in the aortic track. In embodiments, tooling to construct the flange 2515 can include top plates and nested contoured rings for producing the transition zone. In embodiments, before shape setting of the flange, the petals (or end loops of the flange) of the flange can be braided to be shorter at the D-shape perimeter than the other petals or ends of the flange, for example, to accommodate the LVOT or aorto-mitral curtain.

In some embodiments, the layer of material 2530 may cover at least a portion or all of the flange 2515. In some embodiments, the top plate 2545, the first contoured ring 2535, and the second contoured ring 2540 may each have an underside surface, which in some examples may be covered by the layer of material 2530. In some embodiments, at least one of the underside surface of the top plate 2545, the underside surface of the first contoured ring 2535, and the underside surface of the second contoured ring 2540, and due in part to the braiding structure and orientation described herein, may be configured to contact at least a portion of native tissue so as to reduce gaps between the native tissue and the valve replacement incorporating the flange 2515.

In some embodiments, the first contoured ring 2535 may have a particular pattern or contours and the second contoured ring 2540 may have a pattern or contours, which may be distinct from each other. In addition, in some embodiments, the second contoured ring 2540 may have an outer edge and an inner edge. In some examples, the outer edge of the second contoured ring 2540 may be contiguous to the first contoured ring 2535 (and, e.g., an inner edge thereof). In some examples, the inner edge of the second contoured ring 2540 may be contiguous to the cylindrical mandrel 2555 or heart valve adapter frame.

As shown in FIGS. 25D and 25G, the flange 2515 may also include one or more (e.g., three, as shown) tabs 260 to assist in holding the MLS in place.

### ReValving Method Embodiment

Described herein is also a method of replacing a valve, which may be referred to as “revalving.” ReValving method embodiment may present advantages over existing valve replacement methods. For instance, the common “valve-on-valve” procedure basically crushes and destroys an old valve in order to install a new one. While such a method is better than no option for patients in need of a valve replacement, it entails certain disadvantages. Specifically, space is extremely limited across heart leaflets and annular regions. Thus, stacking multiple devices or structures across the leaflets or in the annulus of a native heart valve results in a loss of effective area of the heart for treatment and natural heart function.

The limited area of native tissue in and surrounding the mitral valve area may be preserved by methods described herein. In addition, in accordance with aspects of this disclosure explained below, revalving is more likely to be safely performed for a younger population group, thereby expanding the current projected number of patients that may currently be treated with TMVR.

One revalving method embodiment for replacing a valve (e.g., 2215) may involve both transapical access and transeptal access. A replacement valve or replacement MLS may be delivered transeptally to the heart valve that will be replaced. For instance, as shown in FIGS. 22A-B, and in FIG. 26, a guidewire 2605 may be inserted and enter from transeptal puncture 2610 and may be used to advance the catheter 2615 and new MLS (not shown).

Unlike the delivery method embodiment described above that includes sending an entire device embodiment 2215, only a new MLS/replacement valve itself may require sending inside the sheathe or catheter 2615 and through the transeptal puncture 2610.

Some revalving method embodiments may include maintaining vigilance while advancing to ensure the existing leaflet structure 2625 has been cleared and/or that the guidewire 2605 has not inadvertently been placed down a wrong path within the native heart. In some methods embodiments, that sheathe or catheter 2615 may then be positioned over the existing implant within the native heart valve.

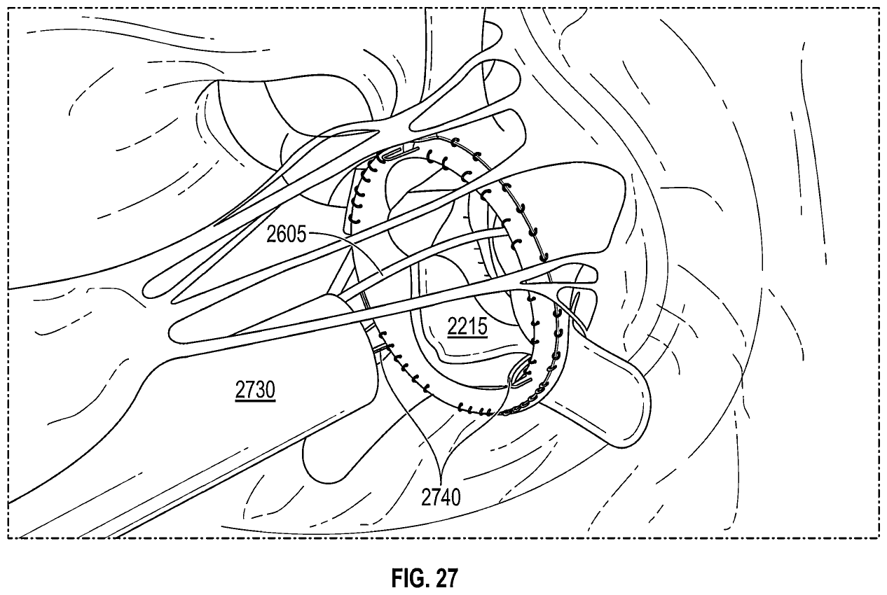

As shown in FIG. 27, the guidewire 2605 may then be exteriorized (transapically) from the transapical sheath 2730, which may create a line or “rail” from the transeptal side to the transapical side. This guidewire/rail 2605 may permit removal of the old MLS 2215 and delivery of the new MLS (not shown) off the same wire. Using the same rail for both removal and delivery ensures that both actions occur in the same plane, entailing advantages that will be readily apparent to the ordinarily skilled artisan. For example, replacement using that rail 2605 permits operations from both opposite ends of the rail 2605. It also entails a less invasive procedure with less abrasions and cuts into native tissue, while eliminating the danger of crossed wires.

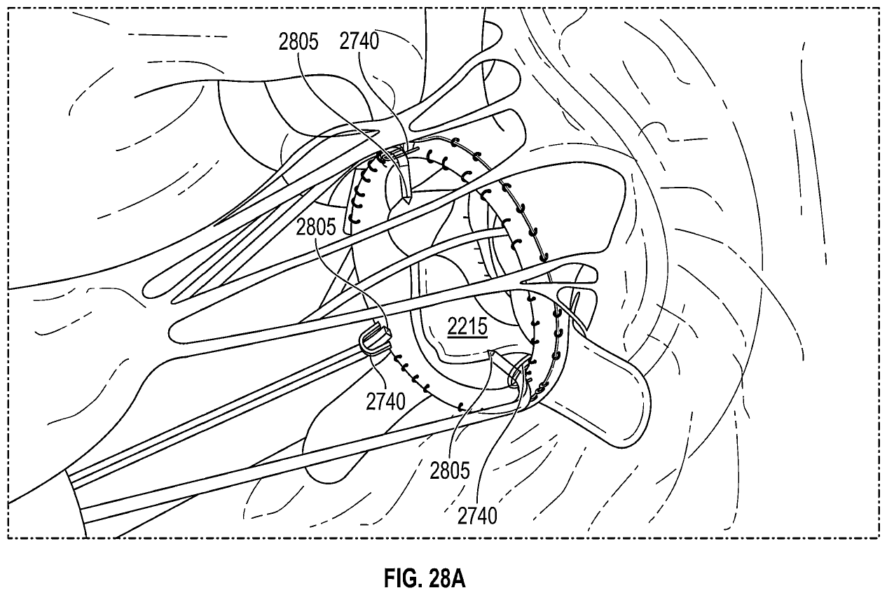

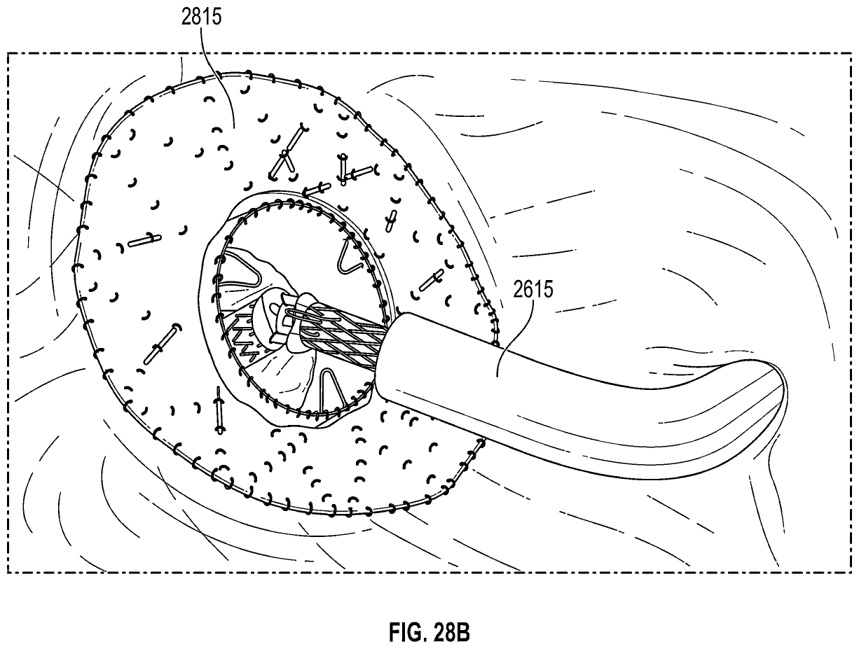

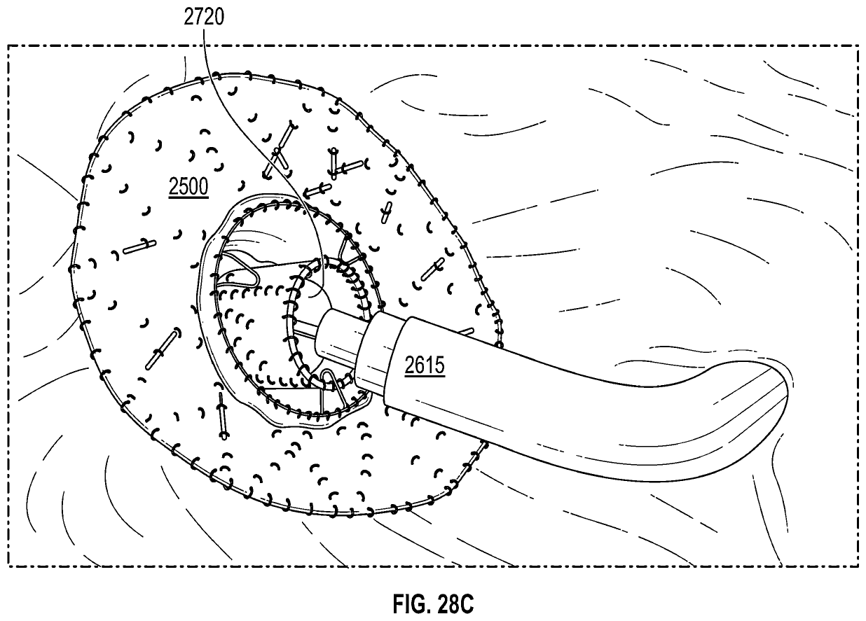

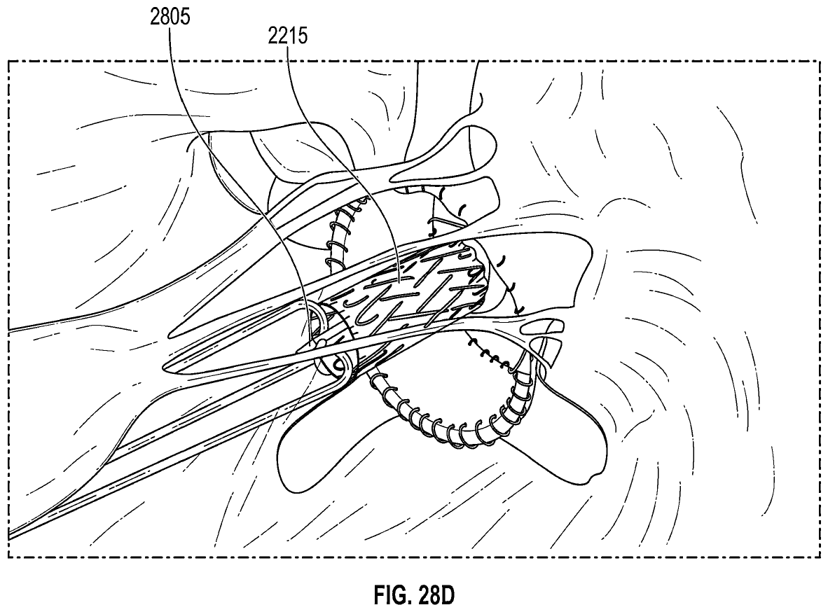

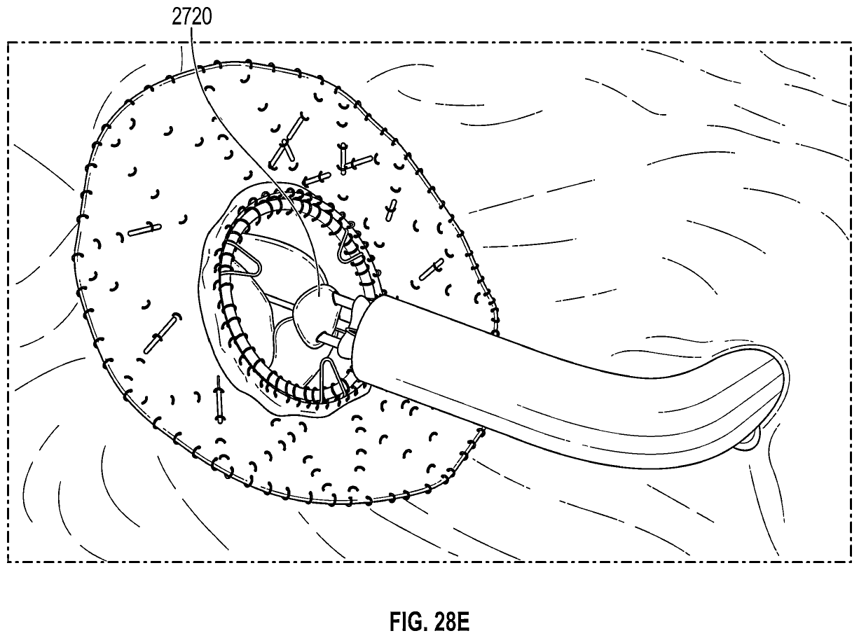

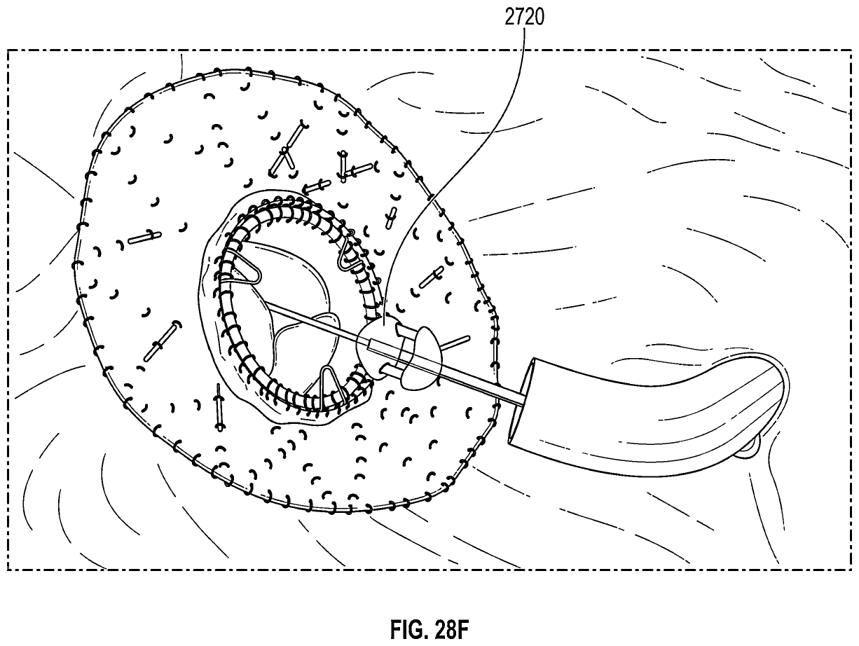

Thus, in addition to transeptal access to the implant, some revalving embodiments may also include transapical access up to the implant. Some embodiments may involve determining that the revalving devices (such as MLS remover 2805 shown in FIG. 28A) has not or does not become intertwined in chordae 2735, by for example advancing the transapical sheath 2730 up towards the valve opening near the annulus.

Some revalving embodiments may also include orienting markers on the catheter 2715 with tabs 2740 of the old valve/MLS 2215. Such orienting or lining up may be performed using, e.g., laroscopy/fluoroscopy, or through “snaring” methods that may involve “loop-and-lassoing,” and other methods known to those of ordinary skill in the pertinent arts.

FIG. 28A shows grabbing of tabs 2740 on the old MLS 2215. In some exemplary (and not exhaustive or limiting) ways of removing the old MLS 2215, markers may be placed on the tabs 2740, and also features of the valve 2215, and on hooks or graspers 2805 of a grabber 2810 (as shown in FIG. 28G). One non-limited embodiment (shown) may feature three tabs 2740 for removing the old MLS 2215. Other non-limited embodiments (not shown) may feature one tab configured to be grabbed. Other embodiments may also be implemented in the revalving method embodiments described herein for transapical removal of the old MLS 2215, and nothing in this disclosure is intended to limit method embodiments to a single way of transapical removal.

Once in proper position, the graspers 2805 may be released and inserted into spots of the tabs 2740 to snare or grab them. Some method embodiments may also include turning on suction to catch any unwanted and potentially harmful debris, to prevent cerebral embolization and/or a stroke from occurring. Accordingly, the graspers 2805 may be ready to pull the tabs 2740 to remove the old MLS 2215. In some embodiments, such steps may be performed by a first operator.

At the same time that the old MLS/valve 2215 is being prepared for removal, on the transeptal or atrium side, and as shown in FIGS. 28B-C, new leaflets as part of a new MLS 2720 may be placed into position for delivery/installation. In some embodiments, such steps on the transeptal side may be performed by a second operator.

Next, the graspers 2805 may pull the tabs 2740 to remove the old MLS 2215, as shown in FIG. 28D, at essentially the same time as the new MLS 2720 is advanced into place, as shown in FIG. 28B, and then the sheathe 2615 may be released, as shown in FIG. 28C and FIG. 28E, allowing the new MLS 2720 and flange 2815 to expand radially, as shown in FIG. 28F. In some examples, the replacement of the old MLS 2215 with the new MLS 2720 may occur rapidly to reduce complications such as regurgitation. Thus, the old valve MLS 2215 may be pulled out/down from the transapical side as the new valve MLS 2720 is advanced into place from the transeptal side, thereby “revalving.”

Some such revalving embodiments may also involve pacing (e.g., rapid pacing and stilling the heart) or non-pacing and slowing down the heart (as may be used for valve-on-valve procedures). Some method embodiments may include using backstop tabs (to backstop the new MLS 2720 from entering too far into the atrium) and utilizing positive pressure.

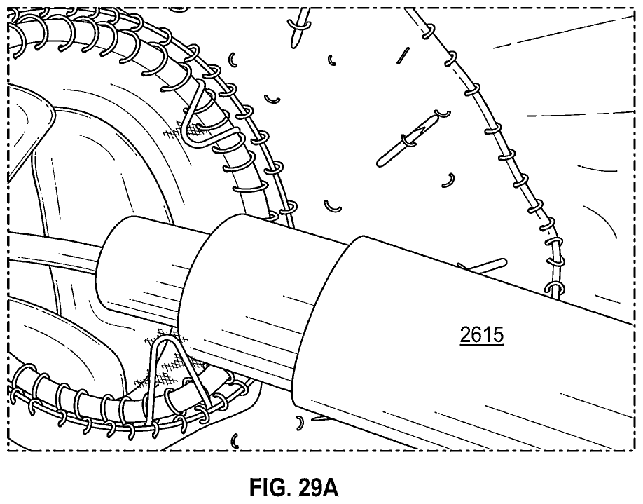

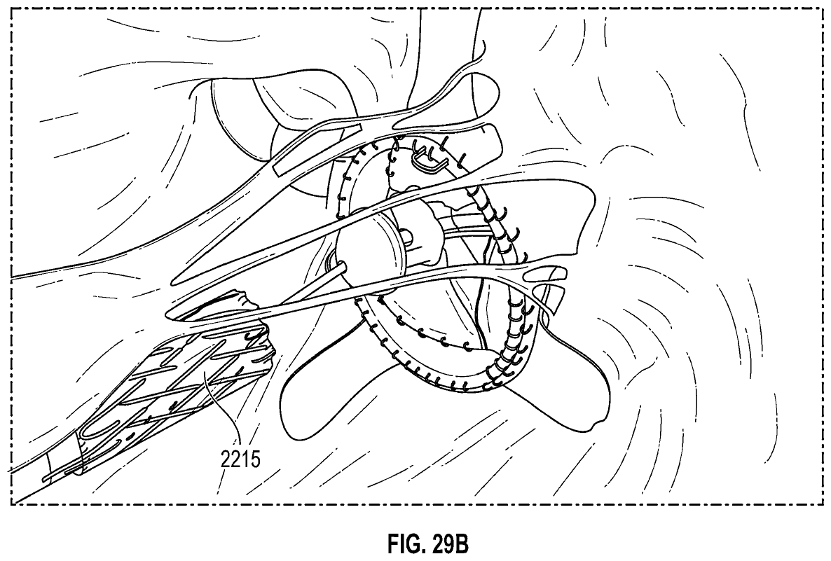

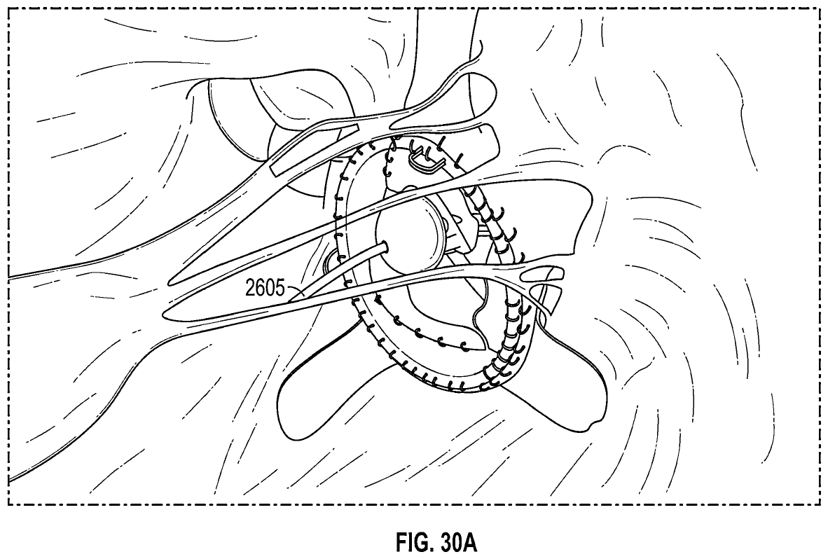

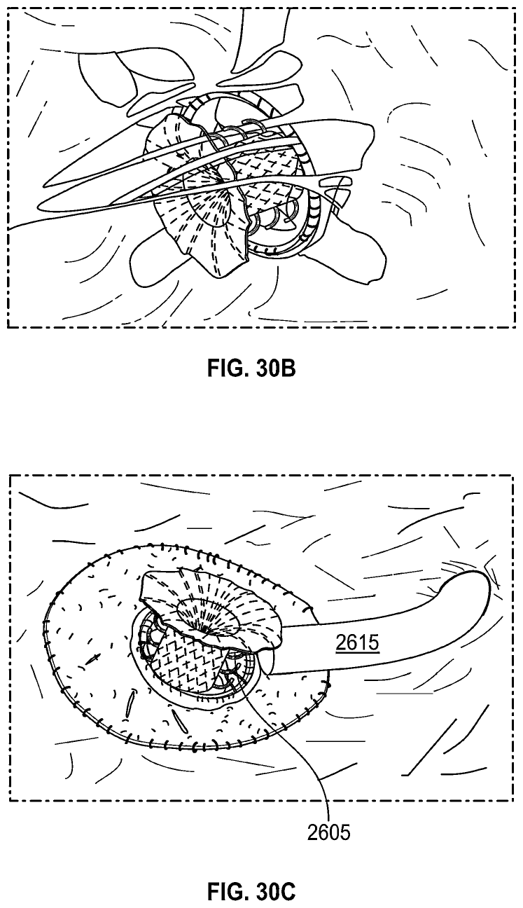

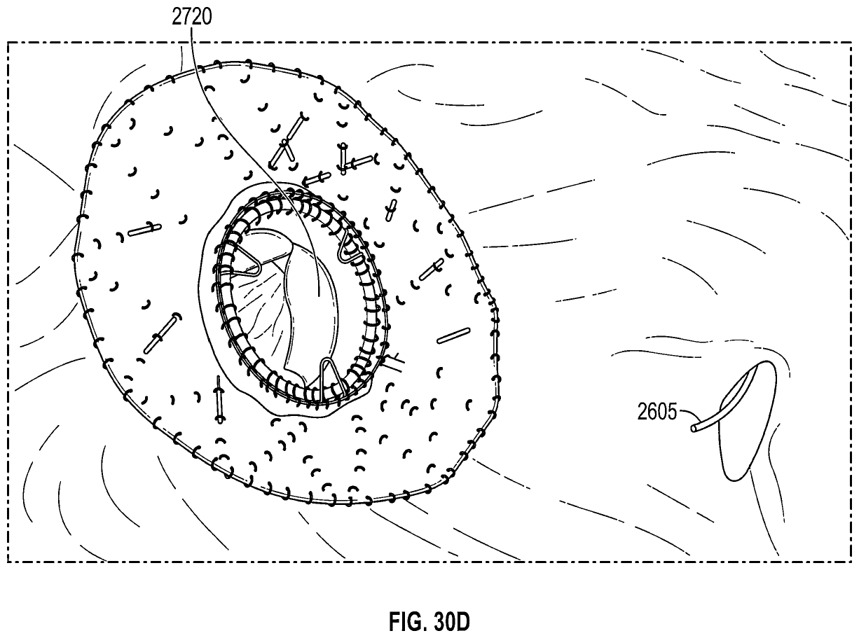

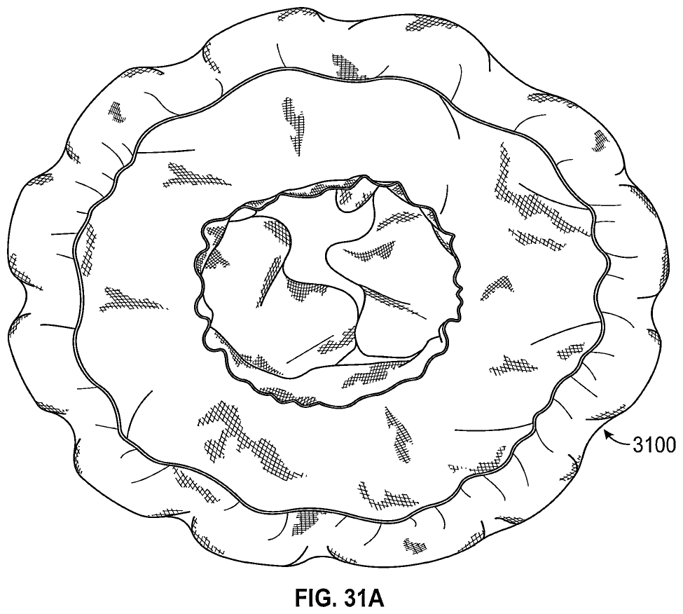

Specifically, after the old MLS 2215 and potentially related-structure is removed transapically as shown in FIG. 29B, while catheter 2615 and guidewire 2605 are also removed transeptally, as shown in FIGS. 30A-D. One benefit of revalving using the two-piece system is that replacing the old MLS/interior valve 2215 may not require pulling off native tissue as collateral damage. While it may entail pulling off fabric of the adapter, the missing fabric may immediately or promptly be scaled back with the new MLS 2720. Thus, the adapter may remain in place as in some aspects of an anchoring system (which may functionally serve as a new annulus) while just old and new MLS 2215, 2720 (e.g.) are exchanged.

In some embodiments, the new MLS 2720 may have a different design from the old MLS 2215 in one or more aspects. For example, the predetermined oversizing of the new MLS 2720 may be different, based on different needs, objectives, or adapter dimensions that have changed over time. Thus, embodiments of methods described herein may feature customizable radial force.

In further embodiments, the revalving may occur via only a transseptal approach on the atrial side of the heart with the old MLS being removed by and new MLS being inserted through the same transeptal catheter system in the atrium of the native heart, without need for a transapical catheter approach from the ventricle side. In other embodiments, instead of removing the old MLS, a new MLS is inserted into the old MLS through a “valve-in-valve” procedure that can be done cither transapically or transeptally. In these embodiments, the new MLS is designed with an oversized, shape-set, helical braid pattern with sufficient outward radial force when deployed within the existing MLS, so that the new MLS frame will push the old MLS out of the way and create space within the annulus for the new MLS to function properly within the annulus.

### Anchoring Embodiments

The following describes several embodiments, which may still feature one more of: (1) supra-annular securement; (2) sub-annular securement; (3) leaflet securement; and/or (4) radial force securement. Each of these embodiments may either be embodied as a One- or two-piece system.

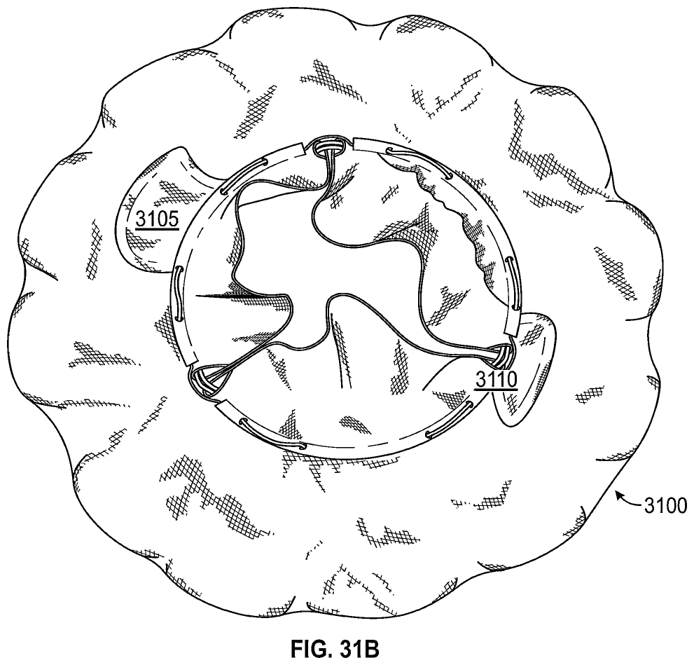

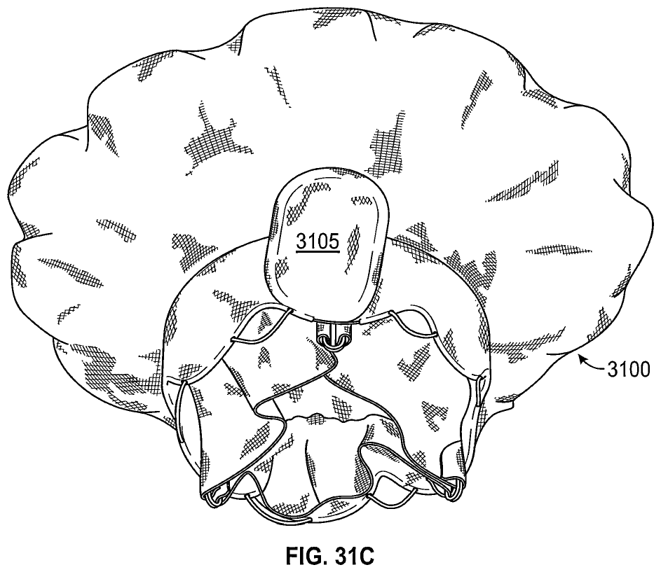

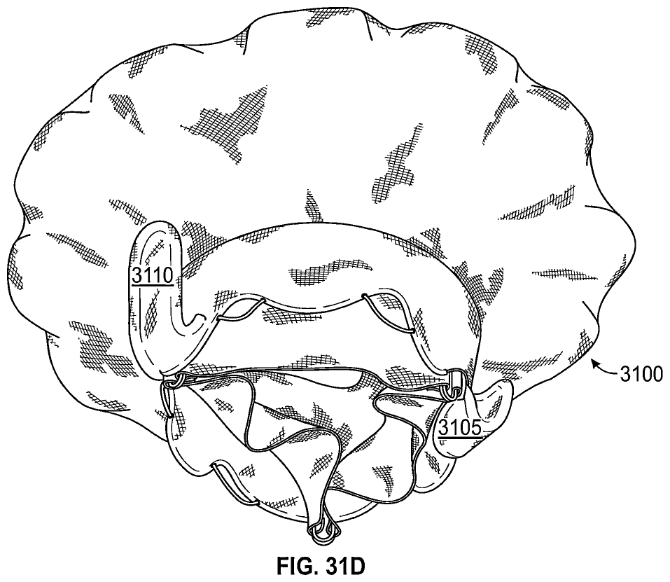

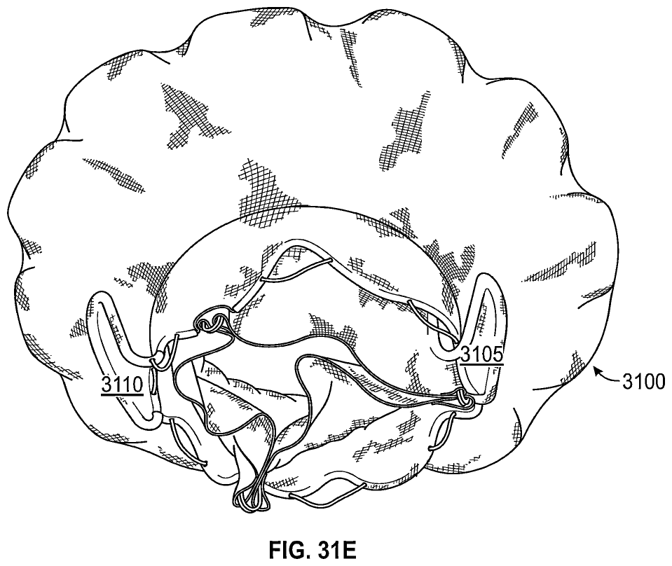

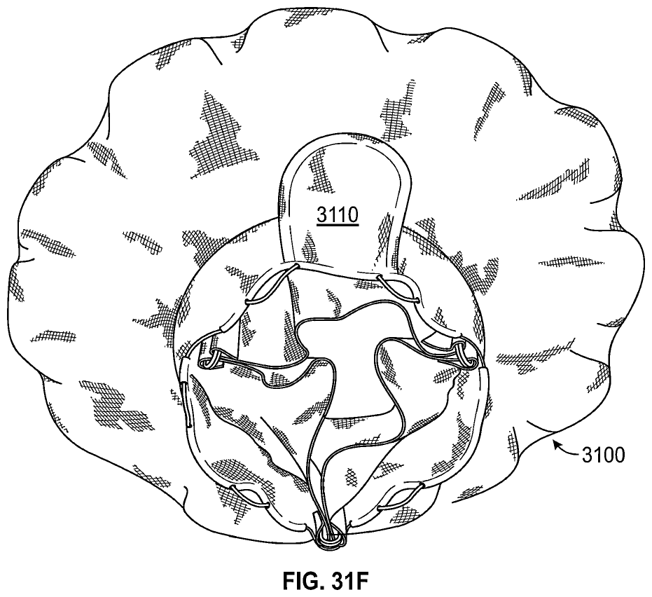

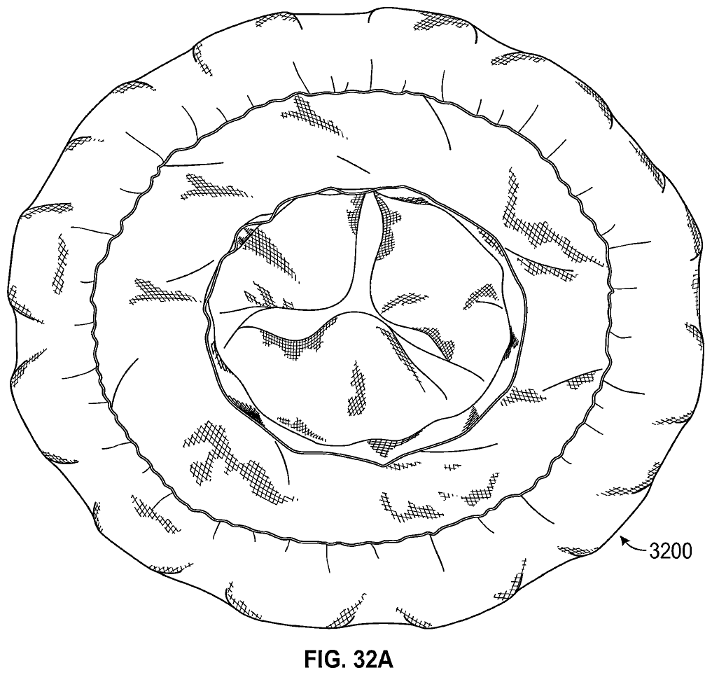

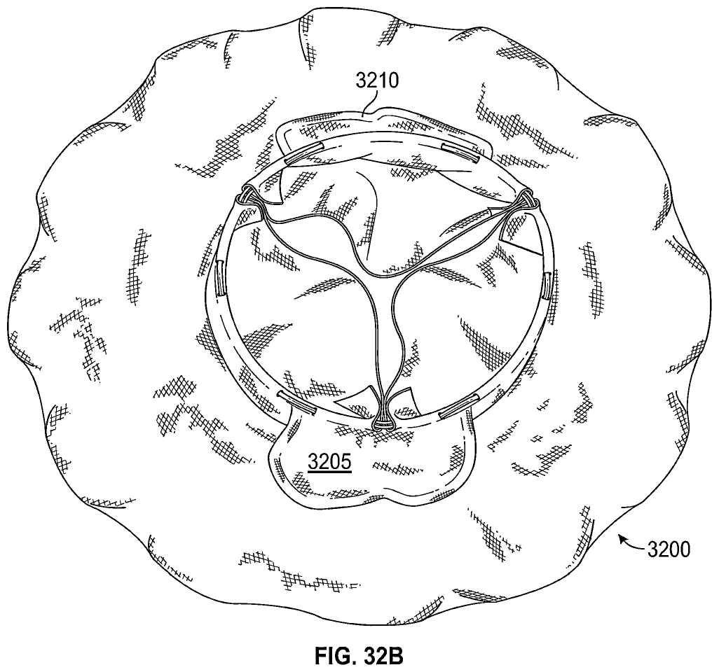

FIGS. 31A-F generally illustrate an embodiment of a valve replacement as disclosed herein. FIGS. 31A-F show a device embodiment 3100 using two anchor features or clips 3105, 3110. In some examples, the device embodiment 3100 may function similarly with regard to delivery and operation as device 2215 shown and described above, but without anchors and the steps described above associated with such anchors. For example, two leaflet clips 3105, 3110 may be configured to attach and/or secure to different regions of native tissue, such as leaflets.

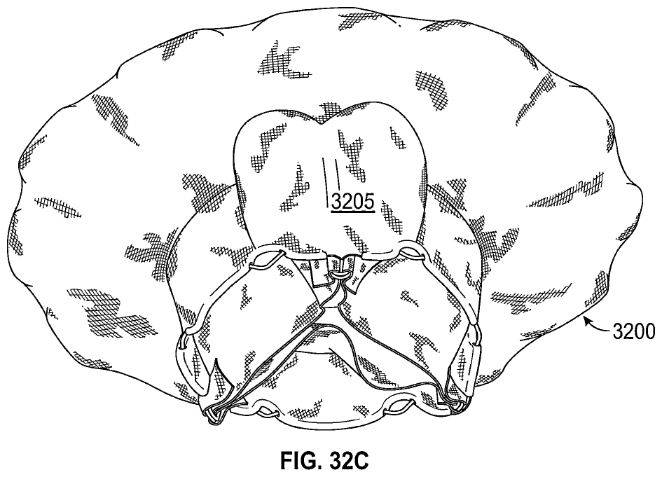

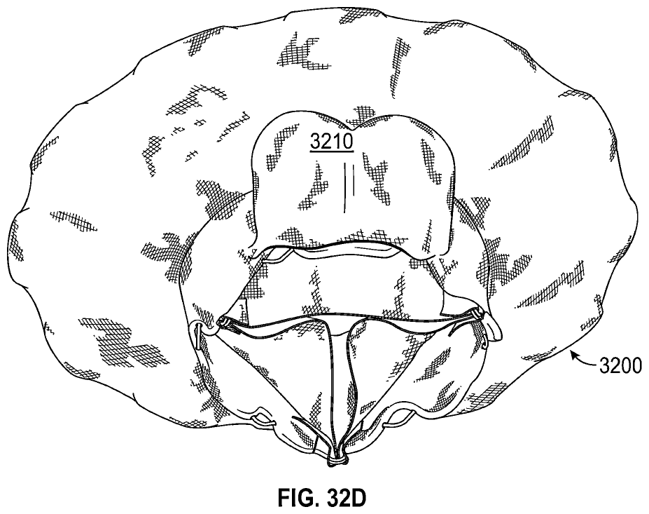

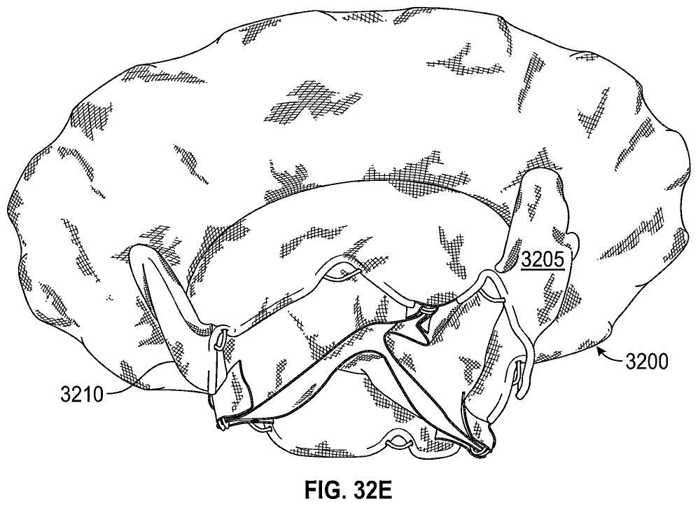

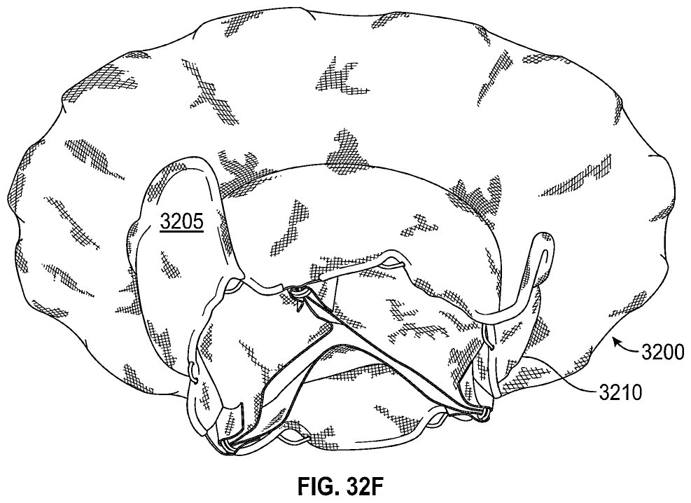

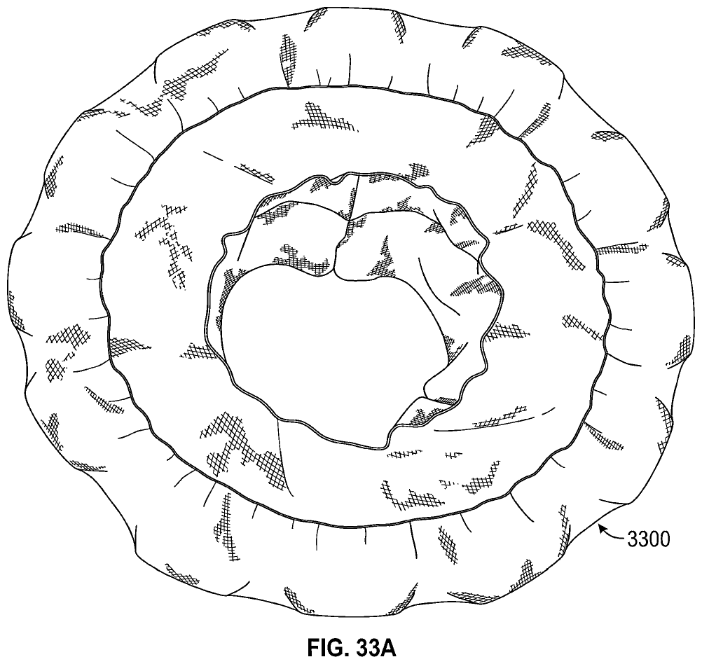

FIGS. 32A-F generally illustrate an embodiment of a valve replacement as disclosed herein. FIGS. 32A-F show a device embodiment 3200 using two clips 3205, 3210 as described above without anchor struts, but where the two leaflet clips 3205, 3210 are each substantially wider (and in some examples, substantially twice as wide) as the two leaflet clips 3105, 3110 shown with regard to device 3100. For example, two leaflet clips 3205, 3210 may be configured to attach and/or secure to different regions of native tissue, such as leaflets. For instance, in some embodiments the two wider leaflet clips 3205, 3210 may grab or secure to a much larger region (or larger regions) of the native leaflets, such as the A2 anterior region or the P2 posterior region.

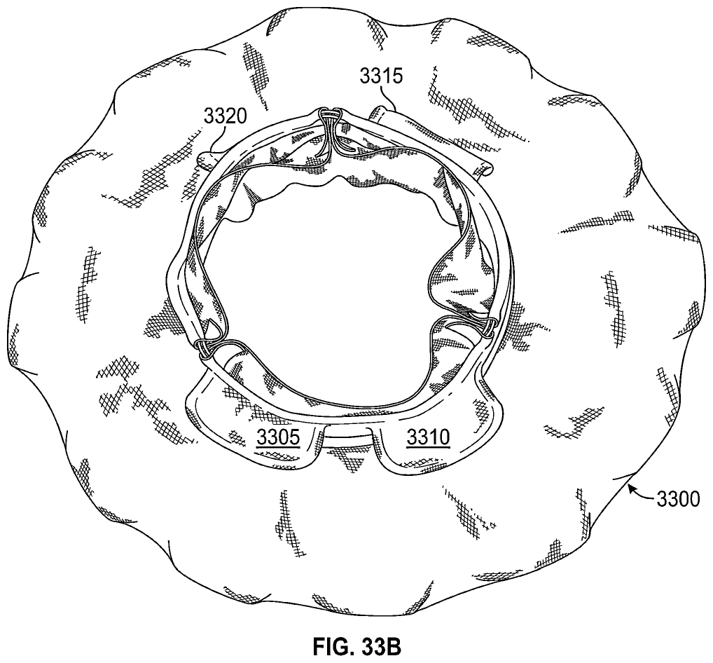

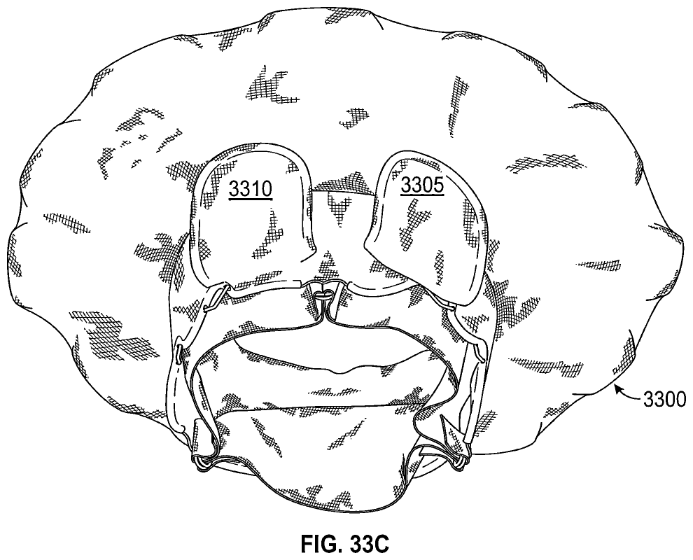

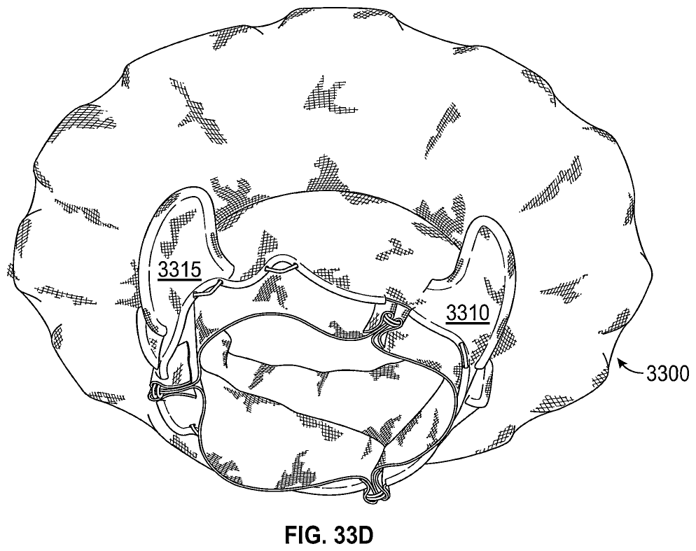

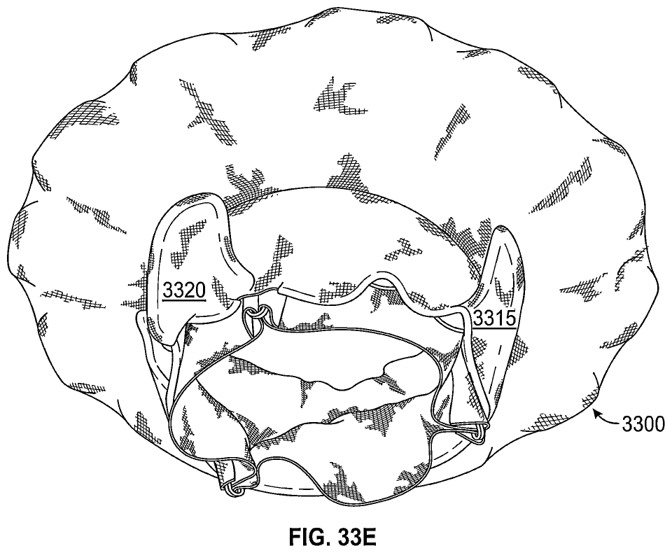

FIGS. 33A-F generally illustrate an embodiment of a valve replacement as disclosed herein. FIGS. 33A-F show a device embodiment 3300 having four clips 3305, 3310, 3315, 3320 but without any anchors. In some examples the four clips 3305, 3310, 3315, 3320 may each be configured to attach and/or secure to a different predetermined area of the native tissue, such as leaflets and areas thereof. In some examples, at least two of the four leaflet clips 3305, 3310, 3315, 3320 may be configured to secure the anterior leaflet region (for example, 3320 and 3315 may attach to either side of the A2 anterior leaflet region) and the posterior leaflet region (for example, 3305 and 3310 may attach to either side of the P2 posterior leaflet region).

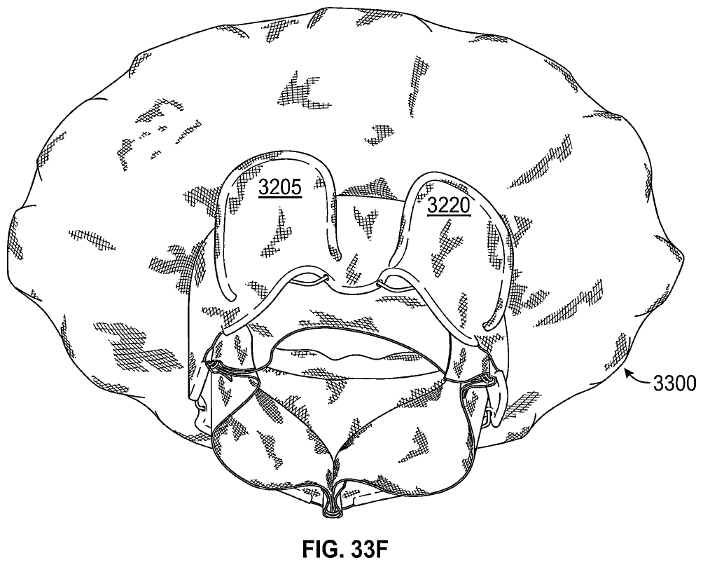

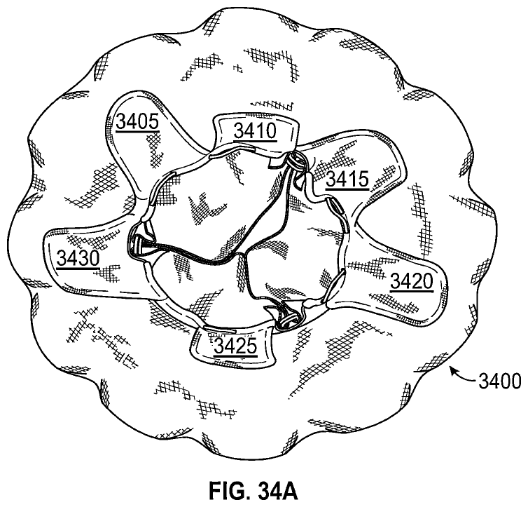

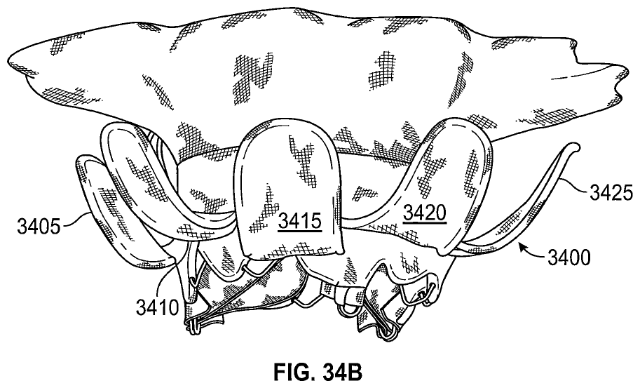

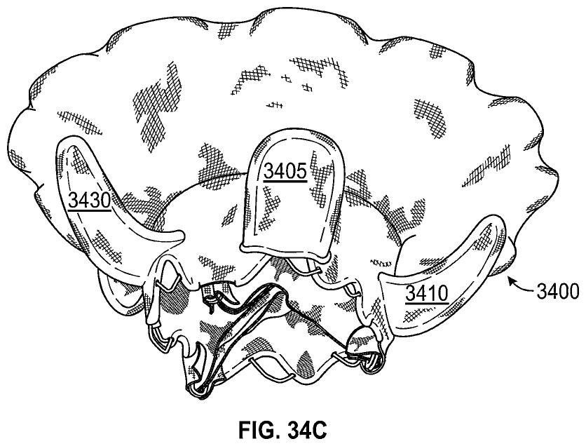

FIGS. 34A-C generally illustrate an embodiment of a valve replacement as disclosed herein. FIGS. 34A-C show a device (i.e., “Dragonfly”) embodiment 3400 having two leaflet clips 3410, 3425 and four anchor struts—two struts on the medial side 3405, 3430 and two on the lateral side 3415, 3420.

In embodiments, each of the clips and anchors may each be configured to anchor to a different predetermined area of the native tissue, such as leaflets, inter-commissural areas, and underneath chordae. In some examples, the clips 3410, 3425 may be configured to clip to particular regions of the native leaflets, e.g., may envelope and attach to the native anterior and posterior leaflets in the A2 and P2 regions, respectively, and the anchor struts may rest near and under the anterior leaflets (anchors 3405 and 3415) and posterior leaflets (anchors 3430 and 3420). In embodiments, each of the clips and anchors may each be configured to anchor to a different predetermined area of the native tissue, such as leaflets, inter-commissural areas, and underneath chordae. For instance, the clips may be configured to clip to particular regions of the native leaflets, e.g., the anterior 2 region or the posterior 2 region, etc., respectively. By way of further example, the anchors may be configured to rest in and anchor against the native heart trigone areas, inter-commissural areas, underneath leaflets, and/or underneath chordae near the annulus of the native heart valve being treated (e.g., the mitral valve, tricuspid valve, or aortic valve).

### Implant Delivery Devices

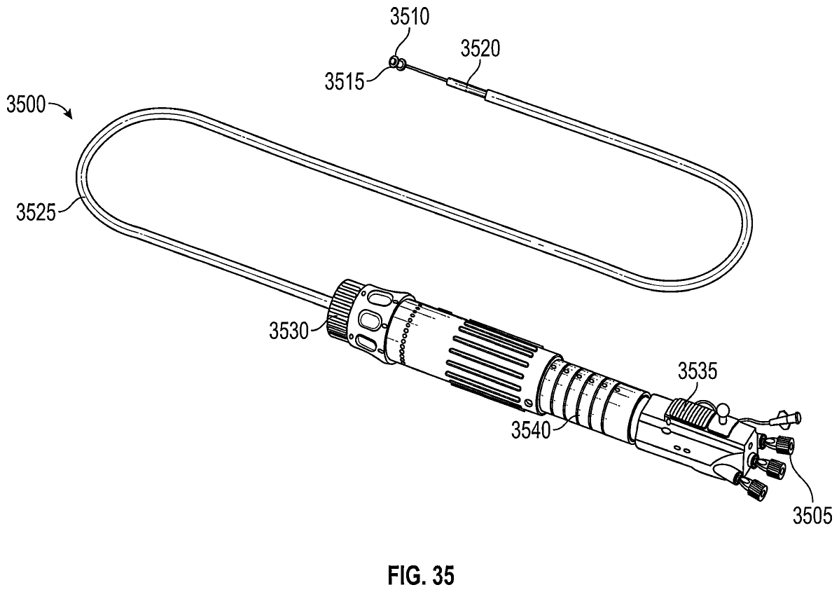

FIG. 35 shows an implant delivery catheter embodiment 3500, in accordance with aspects of this disclosure. The catheter 3500 may have a proximal end 3505 and a distal end 3510. In some embodiments, the distal end 3510 may include a nose cone 3515. Below the nose cone 3515, in some embodiment, may be a steerable end of the catheter system.

In between the distal end 3510 and the proximal end 3505, and connected to the steerable end 3520, may be a liner 3525. In some embodiments, the liner 3525 may be connected on the proximal end 3505 side to an implant steering knob 3530. In some embodiments, the proximal end 3505 may also include a pull-wire slider 3535. And in some embodiments, between the pull-wire slider 3535 and the implant steering knob 3530 may be an implant shaft depth indication 3540.

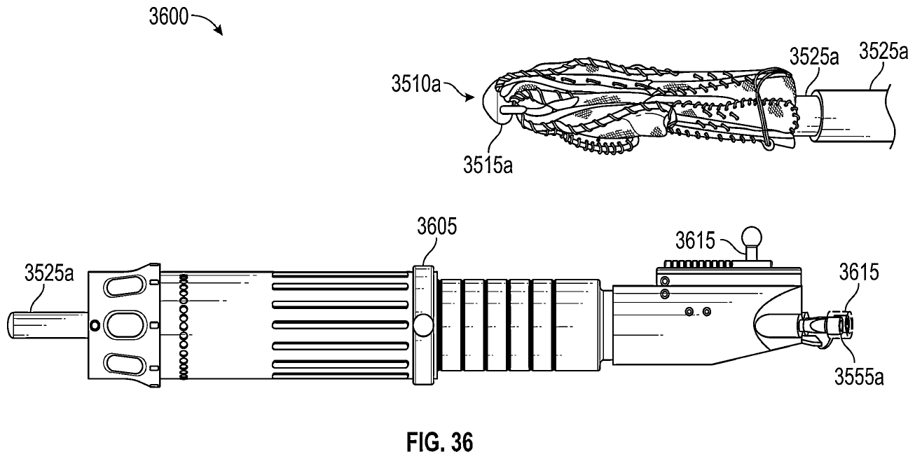

FIG. 36 shows a tethered implant catheter embodiment 3600 in accordance with aspects of this disclosure. The tethered implant catheter embodiment 3600 may be similar in some respects to the implant catheter embodiment 3500 of FIG. 35. For example, the catheter 3600 may also have a proximal end 3505a and a distal end 3510a. The distal end 3510a may include a nose cone 3515a, which may be connected (towards the proximal end 3505a) to a steerable catheter 3520a. The steerable catheter 3520a may be connected (towards the proximal end 3505a) to a liner 3525a. In some embodiments, the liner 3525a may be connected (towards the proximal end 3505a) to a guide insertion lock 3605. In the direction of the proximal end 3505a from the guide insertion lock 3605 may be a pull-wire lock 3610 in some embodiments. And towards the proximal end 3505a from the pull-wire lock 3610 may be a suture-locking solution 3615.

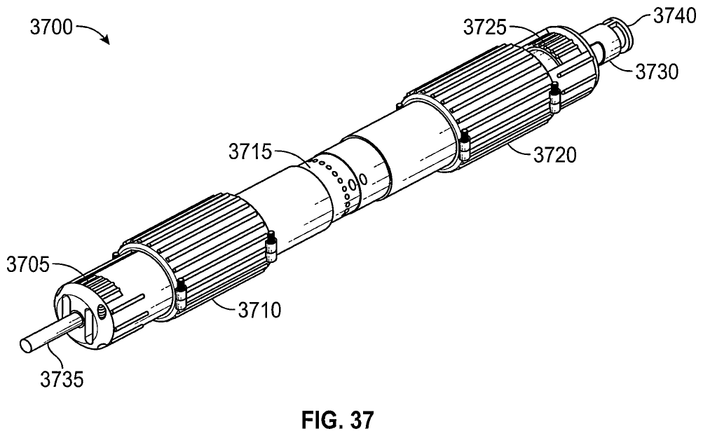

FIG. 37 shows a sheathed implant catheter embodiment 3700 in accordance with aspects of this disclosure. In some embodiments, the sheathed implant catheter embodiment 3700 may have a proximal end 3735 and a distal end 3740. Close to the proximal end 3735 may be sheath steering knob 3705. In some embodiments, the sheath steering knob 3705 may facilitate steering in two directions for a single plane. Towards the distal end 3740 from the sheath steering 3705 may be sheath retrieval handle 3710, which may include a knob.

The sheathed implant catheter embodiment 3700 may also have, close to the distal end 3740, a nose cone retrieval handle 3730 which may include color-coded knobs. Towards the proximal end 3735 from the nose cone retrieval handle 3730 may be a steering knob 3725, which in some embodiments may also be configured for steering in two directions on a single plane. Towards the proximal end 3735 from the steering knob 3725 may be the retrieval handle 3720, which may include a knob.

In between the distal end 3740 (along with the nose cone retrieval features 3730 and/or the chock steering features 3725) and the proximal end 3735 (along with the sheath steering features 3705) may be a cradle clamp 3715. In some embodiments the cradle clamp 3715 may include essentially a single horizontal point or single-point cylindrical loop along the outside of the sheathed implant catheter embodiment 3700.

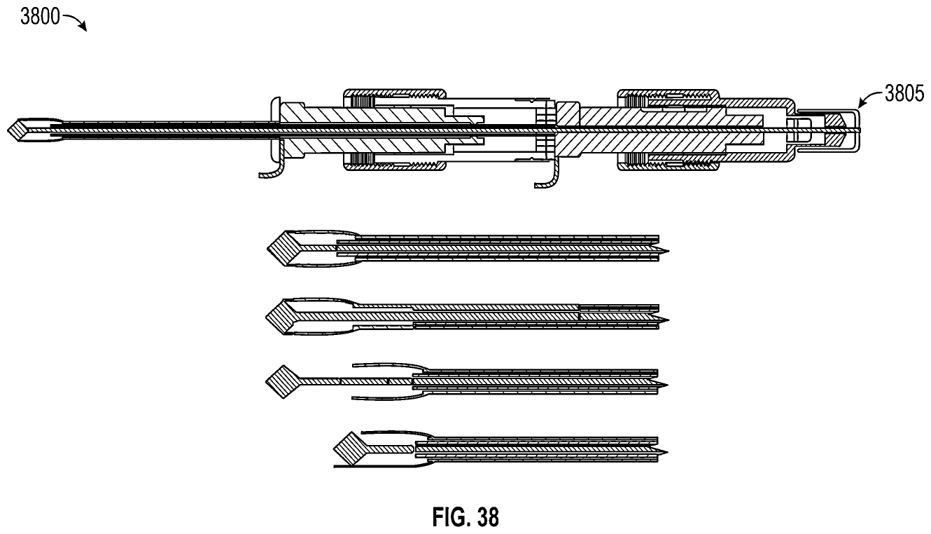

FIG. 38 shows a sheathed implant embodiment 3800 in accordance with aspects of this disclosure. More specifically, FIG. 38 shows, in some respects, how the sheathed implant embodiment 3800 performs movement related to placement and delivery of the valve replacement. The sheathed implant embodiment 3800 may have a nose cone retrieval 3805 on one end.

In some embodiments, the sheathed implant embodiment 3800 may have features for steering in two planes. Additional features may permit unsheathing and/or re-sheathing.

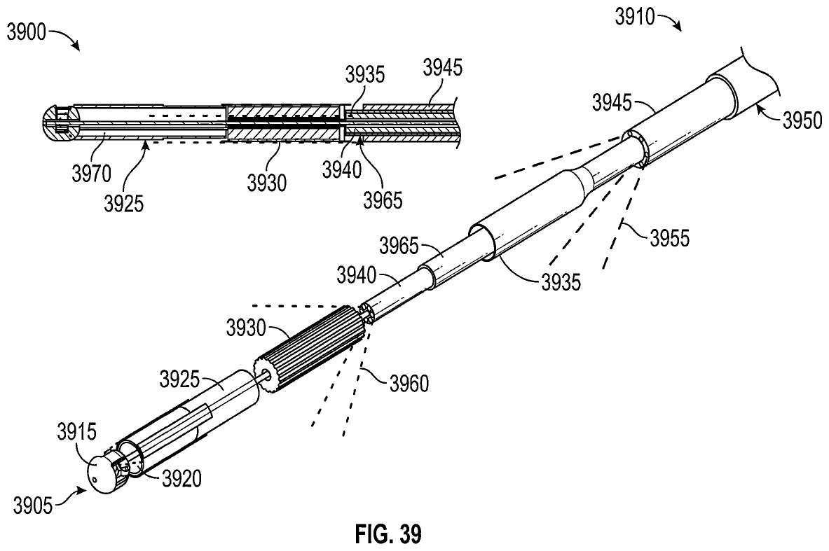

FIG. 39 generally illustrates an embodiment of a two-piece assembly 3900, in accordance with aspects of this disclosure. The two-piece preparation assembly embodiment 3900 may have a proximal end 3910 and a distal end 3905. At the distal end 3905 may be a nose cone 3915, which in some embodiments may be connected to a distal suture 3920.

The two-piece preparation assembly embodiment 3900, in some examples, may have a generally cylindrical shape, and entail several layers and/or internal components. In some embodiments, the layers may vary from one side (e.g., the proximal end 3910) of the two-piece preparation assembly embodiment 3900 to the other (e.g., the distal end 3905), and according to operation and deployments sequence. That is, the cross-section of layers and components of the assembly embodiments 3900 at particular lateral points may differ at different times. In some embodiments, the assembly embodiment 3900 may include a hypotube, through which the distal suture 3920 may run at or near the distal end 3905. Also near the distal end 3905, an outer layer may include an adapter 3925, inside which may be a valve 3930, and over which may be placed a valve sheath 3935.

The assembly embodiment 3900 may also include one or more lumen in between layers, such as an inner multi-lumen 3940 inside the valve 3930, and an outer multi-lumen 3945. Outside the outer multi-lumen 3945 and more likely on the proximal end 3910 may be retractable guide sheath 3950. Within the outer multi-lumen 3945 may be stored a flange suture 3955. The flange suture 3955 may assist in releasing a flange. Within the inner multi-lumen 3940 may be a mid-suture 3960. Over the inner multi-lumen may be a steering catheter 3965. Inside the adapter 3925 may also be a pull-wire 3970 which, in some examples, may assist in performing or triggering operations in accordance with various aspects of this disclosure.

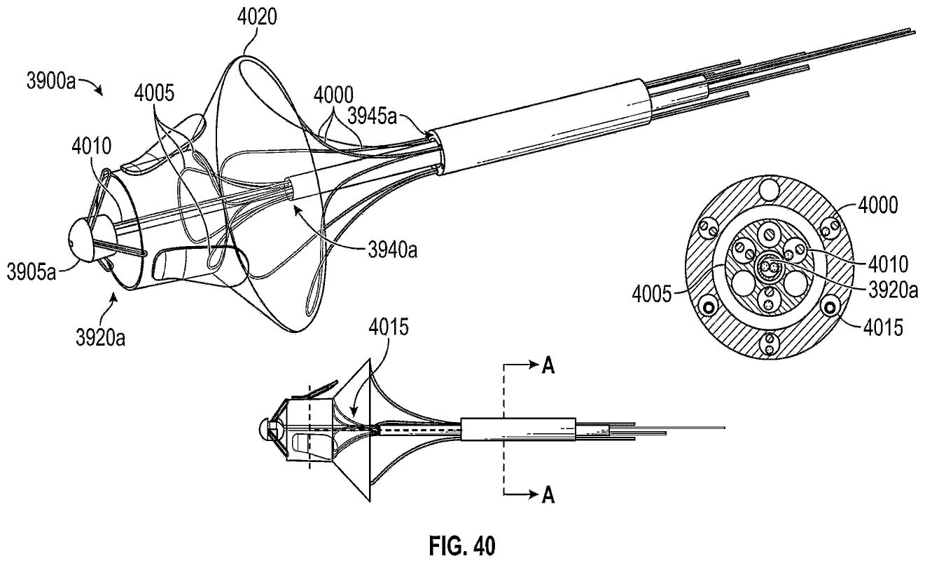

FIG. 40 generally illustrates in more detail some aspects of the two-piece assembly embodiment 3900a of FIG. 39, in accordance with aspects of this disclosure. FIG. 40 shows a two-piece valve tether layout.

In some examples, the outer multi-lumen 3945a may control a flange (and the release or extension thereof) using three sutures 4000 (which may also be referred to as a flange suture 4000). In some examples, the inner multi-lumen 3940a may control the mid-suture using three sutures 4005 (which may also be referred to as a mid-suture 4005). At the distal end 3905a, there may also be a pull-wire 4010. Also at the distal end 3905a may be a distal suture set 3920a (also referred to as a distal suture 3920a), which may pass through the hypotube.

The two-piece assembly embodiment 3900a may connect various sutures 4000, 4005 at an anchor suture loop 4015, which may be located towards the distal end 3905a.

Also described herein is a sequence of use of deploying, e.g., two-piece assembly embodiments 3900, 3900a. The sequence may include: (1) a starting configuration; (2) a prevalving procedure step of advancing an adapter (e.g., adapter 3925); (3) another prevalving procedure step of advancing a valve (e.g., valve 3930); (4) another prevalving procedure step of releasing the valve (e.g., valve 3930); (5) another prevalving procedure step of sizing the valve; (6) deployment procedure, including positioning; (7) a deployment procedure step of extending and deploying anchors; (8) a deployment procedure step of final positioning; (9) and removing deployment of the delivery system and removing the delivery system itself. Some aspects of the sequence may be similar to the valve delivery methods described in the disclosure above. As with other methods described in this disclosure, some sequences of steps and/or details thereof, described below, may be changed in order of operation, may be unnecessary in some embodiments, and/or may be more combined in some aspects.

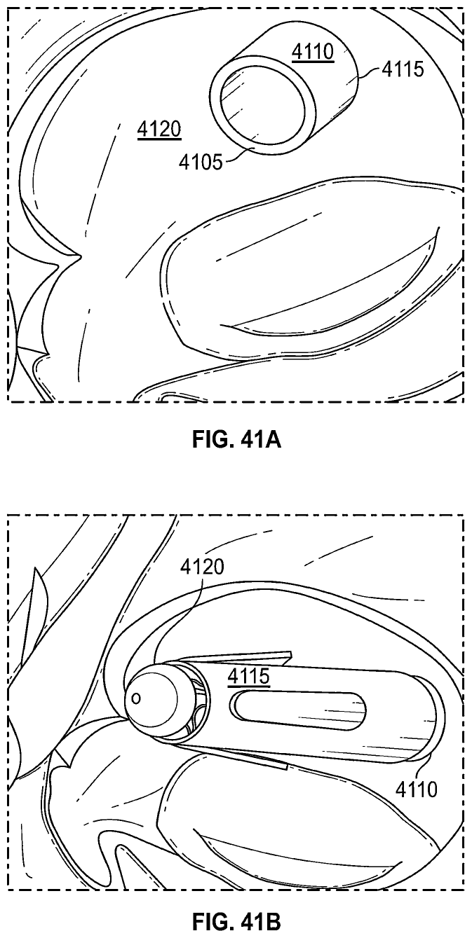

FIG. 41A shows a starting configuration, in accordance with various aspects of this disclosure. As shown in FIG. 41A, the distal end 4105 of the guide catheter/sheath 4110 should cross the septum 4120 to breach the space of the atrium 4125. In some examples, the distal end 4105 of the guide catheter/sheath 4110 may be deflected between 0° and 30° upon entry.

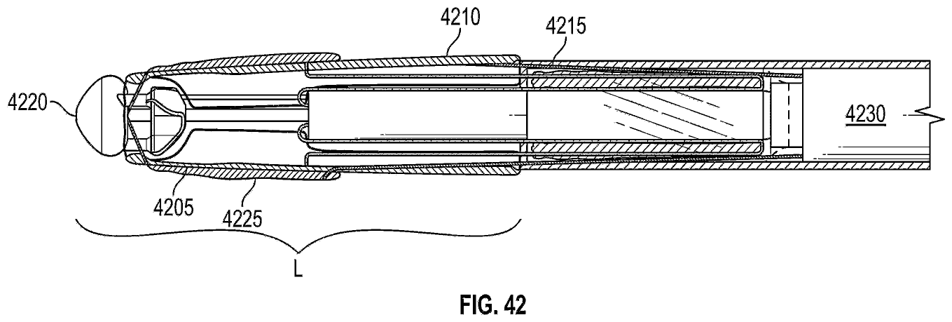

FIG. 41B and FIG. 42 each show a prevalving procedure step of advancing an adapter 4225 (e.g., adapter 3925). As shown in FIG. 41B, the shaft 4115 (which in some embodiments may relate to an adapter) of the delivery system 4120 may also be advanced to the distal end 4105 of the guide catheter/sheath 4110.

Also, as shown in FIG. 42, an adapter 4225 may advance until it is fully within the atrium 4120 (shown in FIG. 41B). In some embodiments, the adapter 4225 may advance approximately in a range of 3-7 cm, and in some examples about 5 cm. In some embodiment, this distance may roughly correspond to the length L of the adapter 4225. In some examples, the length L of the adapter 4225 may be associated with the portion extending from the front of the distal suture 4205 area stretching to behind (e.g., away from the distal suture 4205) a section associated with the mid-suture 4210. During the prevalving/delivery procedure, an MLS or replacement MLS can be loaded on/within the catheter/sheath 4110 on either side of (in front of or behind) the adapter 4225.

Similar in some respects to distal sutures 3920, 3920a of two-piece assembly embodiments 3900, 3900a, adapter 4225 may have a distal suture 4205 that entails a three-suture set. Similar in some respects to mid-sutures 4005, 4005a, adapter 4225 may also have a mid-suture 4210 that has a three-suture set. Similar in some respects to flange suture 3955, adapter 4200 and or guide catheter 4230 may also have a flange (or flange-restraining) suture 4215 that has a three-suture set.

In some embodiments, appropriate tension of the flange-restraining sutures 4215 may assist in permitting a proximal edge of a flange to flare out to a larger diameter than the guide catheter 4230 (which may be similar in some respects to guide catheter 3925).

The flange sutures 4215 may also be tightened to secure the adapter 4225 to the distal end 4220 of the guide catheter 4230. Tension in the flange retaining sutures 4215 may also be used to activate and/or hold in place other features (e.g., tabs or anchor features) and to prevent some features from obstructing a passage for a valve.

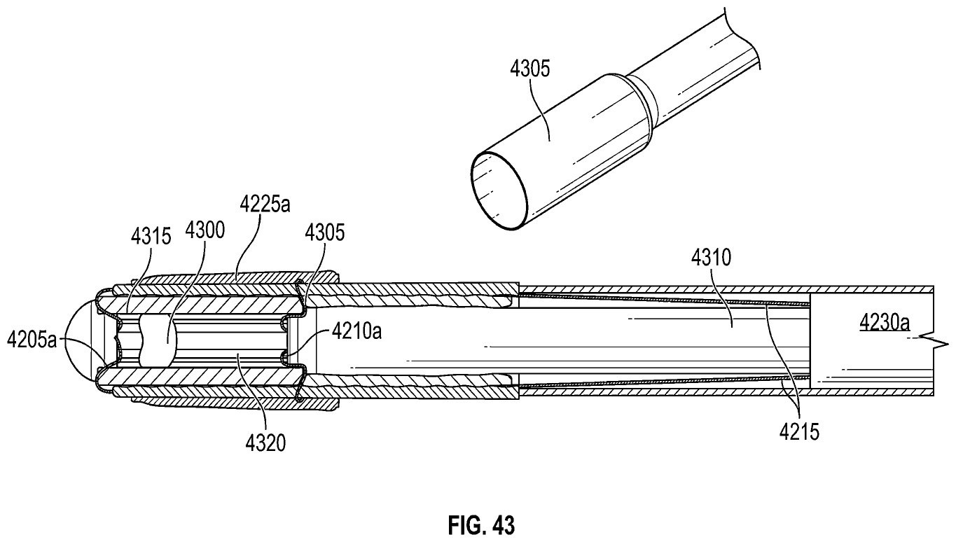

FIG. 43 shows a prevalving procedure step of advancing a valve replacement 4300. The valve 4300 may be coverable by a valve sheath 4305. In some examples, the valve sheath 4305 and sheathed valve replacement may be advanced over the inner multi-lumen 4315 after being pushed by the steerable catheter sheath 4310. In a prevalving embodiment, the steerable catheter sheath 4310 is advanced under a collapsed flange portion of the valve replacement, thereby shortening the effective length of the delivery system and maximizing the space needed in the atrium for effective delivery of the replacement valve. In addition, the midline suture 4210a (from the inner multi-lumen) may be pushed distally as valve 4300 advances. Also, tension may be maintained in the flange-retaining suture 4215a to keep the adapter 4225a firmly positioned on the end of the guide catheter 4230a. The midline suture 4210a may have an appropriate amount of slack, but the distal retaining sutures 4205a may need to be slackened to allow the distal end of the valve 4300 to protrude beyond the distal edge of the receiver 4320. The valve 4300 may be fully inserted into the receiver 4320 portion of the adapter 4225a once the distal end of the valve 4300 is at or beyond the tabs (not shown).

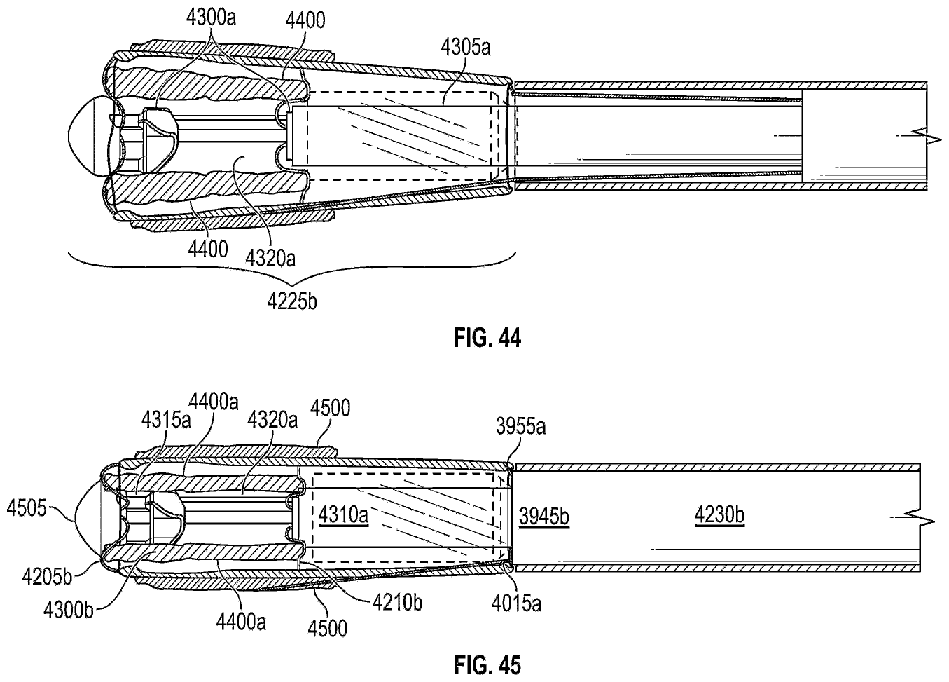

FIG. 44 shows a prevalving procedure that may include releasing the valve 4300a. For example, the sheath 4305a over the valve 4300a may be retracted. In some examples, the sheath 4305a may retract in a range of 3-5 cm, and in some examples about 4 cm. This may allow the valve 4300a to expand within the receiver 4320a portion of the adapter 4225b. The profile 4400 of the valve 4300a may expand outward upon release.

FIG. 45 shows a prevalving procedure step of sizing the valve 4300b. After or as the valve profile 4400 expands as shown in FIG. 44, the midline suture 4210b and the distal suture 4205b may then be tightened to reduce the valve profile 4400a. Based in part on such tightening, the portion of the hypotube between the inner multi lumen 4315a and the nose cone 4505 may be reduced to less and/or a minimum exposed length. In some embodiments, this may involve retracting in a range of 1-4 cm and most commonly in a range of 2-3 cm. The steerable catheter shaft 4310a and inner multi lumen 4315a may be at the proximal edge of the receiver 4320a.

The anchors 4500 may be and preferably remain tethered. In some examples, the anchor-retaining sutures 4015a may need to be tightened. The outer multi lumen 3945b may be advanced and positioned to be flush with the distal end of the guide catheter 4230b. In some examples, such advancing may be in a range of 4-6 cm, and in some examples about 5 cm. The flange retaining sutures 3955a may remain under tension.

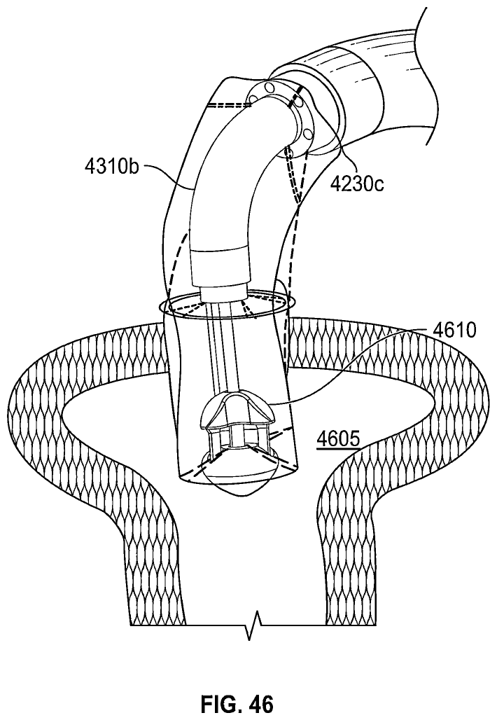

FIG. 46 shows a positioning step of a deployment procedure. For example, the guide catheter 4230c may be deflected to orient the implant 4610 toward the mitral valve annulus 4605. The steerable catheter 4310b may be advanced relative to the guide catheter 4230c and used to provide additional (dual-plane) deflection. In addition, the rotation and axial position of the guide catheter 4230c and steerable catheter 4310b may be adjusted (e.g., by an operator such as a physician) to orient the implant 4610 with the mitral valve annulus 4605.

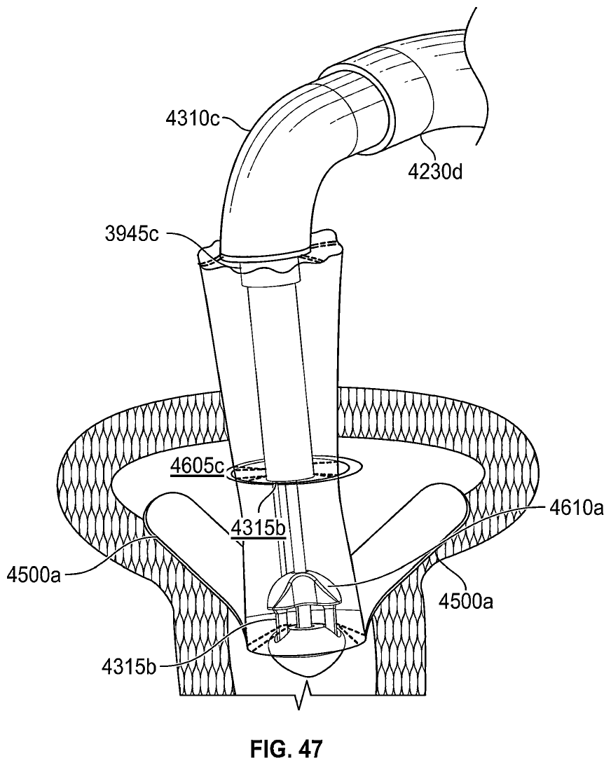

FIG. 47 shows part of the deployment procedure that includes the extension and deployment of anchors 4500a. Once the desired trajectory is achieved, the hypotube and inner and outer multi lumens 4315b, 3945c may extend and be advanced while keeping the steerable catheter shaft 4310c and the guide sheath catheter 4230d stationary to place or locate the implant 4610a at the correct depth within the mitral valve annulus 4605a. Once the desired trajectory and depth of the implant 4610a are confirmed, the anchors 4500a may be released and the alignment to the mitral valve 4605a reconfirmed.

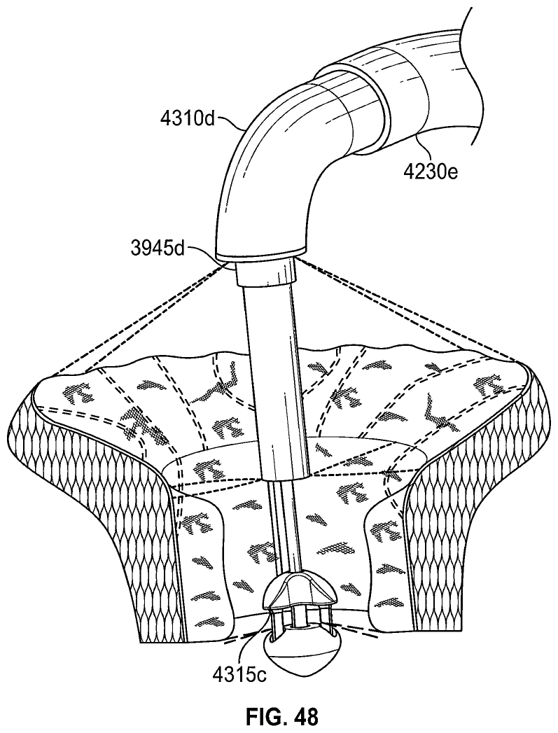

FIG. 48 shows a final positioning step of a deployment procedure. Final positioning details may be determined (by e.g., an operator such as a physician), such as rotation, deflection, and axial translation of the guide catheter 4230e, steerable catheter 4310d, inner and outer multi-lumens 4315c, 3945d and hypotube. Such final positioning may also include releasing suture tension.

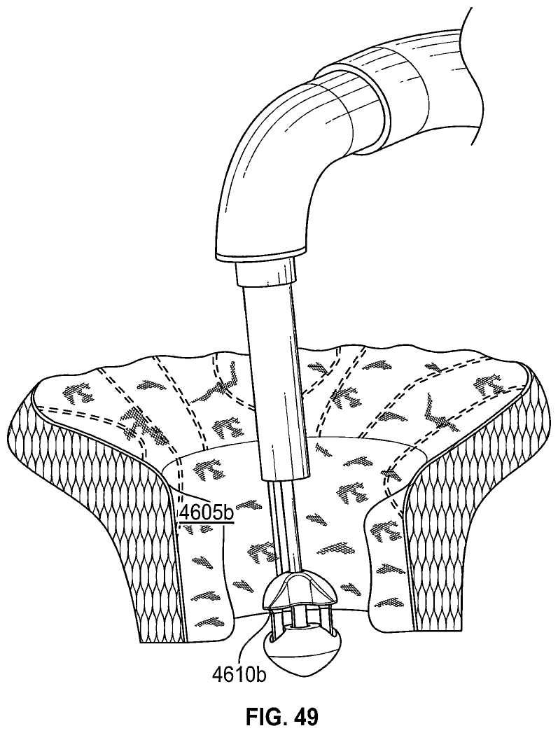

FIG. 49 shows a sequence step of deployment. The implant 4610b is ready for release from the delivery system and for deployment to the native mitral valve annulus 4605b. Such release, in some embodiments, may be triggered by retraction of the pull-wire and suture. After release and installation of the implant 4610b, the rest of the assembly embodiment may be withdrawn in accordance with other aspects of this disclosure.

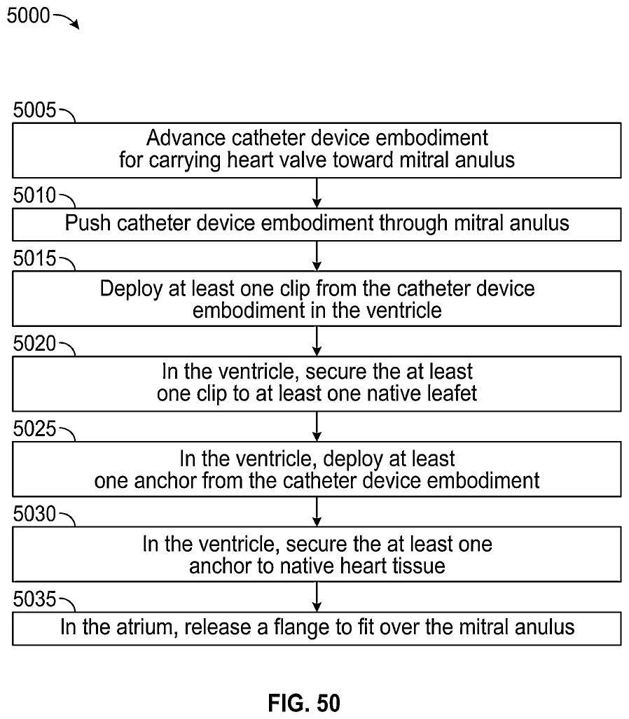

FIG. 50 is a flow diagram generally illustrating a method 5000 of delivering a heart valve, in accordance with aspects of the disclosure. In some embodiments, the method 5000 may include the step 5005 of advancing a catheter device embodiment for carrying heart valve toward the mitral annulus. The method 5000 may include the step 5010 of pushing the catheter device embodiment through the mitral annulus.

The method 5000 may further include the step 5015 of deploying at least one clip from the catheter device embodiment in the ventricle. The method 5000 may further include the step 5020 of, in the ventricle, securing the at least one clip to at least one native leaflet. The method 5000 may further include the step 5025 of, in the ventricle, deploying at least one anchor from the catheter device embodiment.

The method 5000 may further include the step 5030 of, in the ventricle, securing the at least one anchor to native heart tissue. The method 5000 may further include the step 5035 of, in the atrium, releasing a flange to fit over the mitral annulus.

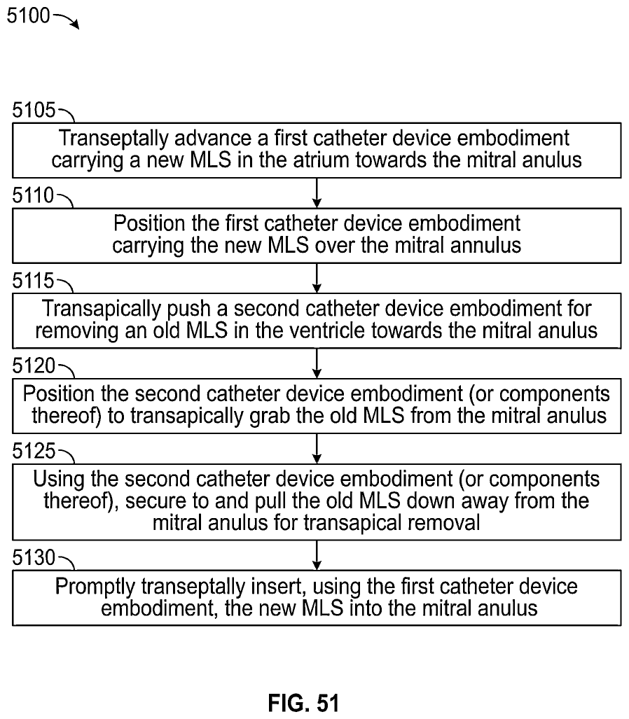

FIG. 51 is a flow diagram generally illustrating a method 5100 of replacing a heart valve that is part of a two-piece assembly, in accordance with aspects of the disclosure. In some embodiments, the method 5100 may include the step 5105 of transeptally advancing a first catheter device embodiment carrying a new MLS in the atrium towards the mitral annulus. The method 5100 may further include the step 5110 of positioning the first catheter device embodiment carrying the new MLS over the mitral annulus.

The method 5100 may further include the step 5115 of transapically pushing a second catheter device embodiment for removing an old MLS in the ventricle towards the mitral annulus. The method 5100 may further include the step 5120 of positioning the second catheter device embodiment (or components thereof) to transapically grab the old MLS from the mitral annulus

The method 5100 may further include the step 5125 of using the second catheter device embodiment (or components thereof) to secure to and pull the old MLS down away from the mitral annulus for transapical removal. The method 5100 may further include the step 5130 of promptly transeptally inserting, using the first catheter device embodiment, the new MLS into the mitral annulus.

One or more of the aforementioned steps (including steps involving both the transapical operations and transeptal operations) may be performed with the assistance of a guidewire, including in some embodiments the same guidewire.

Other embodiments may include combinations and sub-combinations of features described or shown in the several figures, including for example, embodiments that are equivalent to providing or applying a feature in a different order than in a described embodiment, extracting an individual feature from one embodiment and inserting such feature into another embodiment; removing one or more features from an embodiment; or both removing one or more features from an embodiment and adding one or more features extracted from one or more other embodiments, while providing the advantages of the features incorporated in such combinations and sub-combinations. As used in this paragraph, “feature” or “features” can refer to structures and/or functions of an apparatus, article of manufacture or system, and/or the steps, acts, or modalities of a method.

References throughout this specification to “one embodiment,” “an embodiment,” “an example embodiment,” etc., indicate that the embodiment described may include a particular feature, structure, or characteristic, but every embodiment may not necessarily include that particular feature, structure, or characteristic. Moreover, such phrases are not necessarily referring to the same embodiment. Further, when a particular feature, structure, or characteristic is described in connection with one embodiment, it will be within the knowledge of one skilled in the art to affect such feature, structure, or characteristic in connection with other embodiments whether or not explicitly described.

Unless the context clearly indicates otherwise (1) the word “and” indicates the conjunctive; (2) the word “or” indicates the disjunctive; (3) when the article is phrased in the disjunctive, followed by the words “or both,” both the conjunctive and disjunctive are intended; and (4) the word “and” or “or” between the last two items in a series applies to the entire series.

Where a group is expressed using the term “one or more” followed by a plural noun, any further use of that noun to refer to one or more members of the group shall indicate both the singular and the plural form of the noun. For example, a group expressed as having “one or more members” followed by a reference to “the members” of the group shall mean “the member” if there is only one member of the group.

The term “a” or “an” entity refers to one or more of that entity. As such, the terms “a” (or “an”). “one or more” and “at least one” can be used interchangeably herein. It is also to be noted that the terms “comprising”. “including”, and “having” can be used interchangeably.

What is claimed:

1. A method of replacing a native mitral valve, comprising the steps of:
advancing a deployment catheter across a native mitral valve, the catheter carrying an expandable tubular body having a replacement valve, posterior and anterior leaflet clips, medial and lateral struts and an atrial skirt;
noninvasively capturing a native posterior leaflet with the posterior leaflet clip;
noninvasively capturing a native anterior leaflet with the anterior leaflet clip;
noninvasively securing the medial and lateral struts into a native sub-annular space;
expanding the tubular body across the native mitral valve; and
deploying the atrial skirt against a wall of a native mitral annulus and left atrium.

2. The method of claim 1, wherein deploying the atrial skirt against the wall of the native mitral annulus and left atrium resists migration of the tubular body into the native left ventricle and minimizes perivalvular leaks

3. The method of claim 2, wherein deploying the atrial skirt against the wall of the native mitral annulus and left atrium comprises placing a flat portion of the atrial skirt above and on top of the native mitral annulus.

4. The method of claim 3, wherein a native left ventricle outflow track is preserved in part by noninvasively capturing the native anterior leaflet with the anterior leaflet clip prior to noninvasively capturing the native posterior leaflet with the posterior leaflet clip.

5. The method of claim 3, wherein a native left ventricle outflow track is preserved in part by noninvasively capturing the native anterior and posterior leaflets with the leaflet clips.

6. The method of claim 3, wherein noninvasively capturing the native anterior leaflet with the anterior leaflet clip comprises hanging the anterior leaflet clip behind an A2 region of the native mitral leaflets.

7. The method of claim 6, wherein noninvasively capturing the native posterior leaflet with the posterior leaflet clip comprises hanging the posterior leaflet clip behind a P2 region of the native mitral leaflets.

8. The method of claim 7, wherein hanging the anterior leaflet clip behind an A2 region of the native mitral leaflets and hanging the posterior leaflet clip behind a P2 region of the native mitral leaflets permits movement of the native mitral annulus in the anterior and posterior directions when deployed within the native mitral valve.

9. The method of claim 8, wherein securing the medial and lateral struts into the native sub-annular space comprises deploying the medial and lateral struts near the native annulus and placing the medial and lateral struts in between chordae into different sub-annular commissural areas of the native heart.

10. The method of claim 9, further comprising deploying the medial and lateral struts in a native supra-annular space and lowering the medial and lateral struts into a native sub-annular space prior to securing the medial and lateral struts into the native sub-annular space.

11. The method of claim 9, wherein placing the medial and lateral struts in between chordae into different sub-annular commissural areas of the native heart permits movement of the native mitral annulus in the medial and lateral directions when deployed within the native mitral valve.

12. The method of claim 11, wherein noninvasively capturing the native posterior leaflet with the posterior leaflet clip, noninvasively capturing the native anterior leaflet with the anterior leaflet clip, and securing the medial and lateral struts into the native sub-annular space resists migration of the expandable tubular body towards the native left atrium.

13. The method of claim 9, wherein the medial and lateral struts are spaced from each other by no more than 150 degrees.

14. The method of claim 13, wherein the posterior and anterior leaflet clips are symmetrically spaced from each other and located on opposite sides of the expandable tubular body.

15. The method of claim 14, wherein the medial and lateral struts are each spaced from the anterior leaflet clip by a maximum of 75 degrees.

16. The method of claim 15, wherein the expandable tubular body comprises a helically braided nitinol wire frame.

17. The method of claim 16, wherein the helically braided nitinol wire frame of the expandable tubular body is deformable in response to normal cardiac motion thereby preserving natural basal left ventricle function when deployed in the native mitral valve.

18. The method of claim 17, wherein the helically braided nitinol wire frame of the expandable tubular body is shape set to no more than 10% oversize in relation to the native annulus of the native mitral valve.

19. The method of claim 18, wherein a native left ventricle outflow track is preserved by the expandable tubular body when deployed in the native mitral valve in part by a combination of the expandable tubular body being shape set to no more than 10% oversize in relation to the native annulus of the native mitral valve, the expandable tubular body being deformable in response to normal cardiac motion, and the anterior leaflet clip hanging behind, but not pinching, an A2 region of the native mitral leaflet.

20. The method of claim 1, wherein the replacement valve of the expandable tubular body comprises an atrial end, a ventricular end, and a central lumen with replacement leaflets, wherein the replacement valve is cooperatively sized with and configured to seat within the expandable tubular body when deployed in the native mitral valve.

21. The method of claim 20, wherein the replacement valve comprises a valve frame that is separate and removeable from the expandable tubular body when deployed in the native mitral valve.

22. The method of claim 21, wherein the expandable tubular body and the replacement valve each comprise a polymeric membrane covering.

23. The method of claim 22, wherein the medial and lateral struts and posterior and anterior leaflet clips are part of the expandable tubular body and not part of the replacement valve.

24. The method of claim 23, wherein the posterior and anterior leaflet clips and the medial and lateral struts are inclined in the atrial direction.

25. The method of claim 23, wherein the expandable tubular body and replacement valve are unconnected and delivered to the native mitral valve together with the replacement valve seated within the expandable tubular body.

26. The method of claim 23, wherein the expandable tubular body and replacement valve are unconnected and delivered separately to the native mitral valve, with the expandable tubular body delivered to the native mitral valve followed by the replacement valve delivered to the expandable tubular body.

27. The method of claim 23, wherein the expandable tubular body and replacement valve are both self-expanding.

28. A method of replacing a native mitral valve, comprising the steps of:
advancing a deployment catheter across a puncture in a native septum into a native left atrium, the catheter carrying a self-expanding tubular body having a replacement valve, posterior and anterior leaflet clips, medial and lateral struts and an atrial skirt;
advancing the self-expanding tubular body out of the delivery catheter into the native left atrium;
testing a path across the native mitral valve for the self-expanding tubular body by lowering the self-expanding tubular body past the native mitral valve into the native left ventricle, moving the self-expanding tubular body back into the native left atrium, and then lowering the self-expanding tubular body back into the native left ventricle;
noninvasively capturing a native posterior leaflet with the posterior leaflet clip;
noninvasively capturing a native anterior leaflet with the anterior leaflet clip;
noninvasively securing the medial and lateral struts into a native sub-annular space;
expanding the tubular body across the native mitral valve; and
deploying the atrial skirt against a wall of a native mitral annulus and native left atrium.

29. The method of claim 28, wherein noninvasively capturing the native anterior leaflet with the anterior leaflet clip comprises hanging in the A2 region of the native anterior leaflet with the anterior leaflet clip by sliding the anterior leaflet clip across the surface of the A2 region of the native anterior leaflet, and wherein noninvasively capturing the native posterior leaflet with the posterior leaflet clip comprises hanging in the P2 region of the native posterior leaflet with the posterior leaflet clip by sliding the posterior leaflet clip across the surface of the P2 region of the native posterior leaflet.

30. The method of claim 29, wherein the native anterior and posterior leaflets may be noninvasively captured with the leaflet clips in any order or simultaneously, wherein noninvasively capturing the native anterior and posterior leaflets with the anterior and posterior leaflet clips occurs before noninvasively securing the medial and lateral struts into the native sub-annular space and wherein noninvasively capturing the native leaflets with the anterior and posterior leaflet clips and noninvasively securing the medial and lateral struts into the native sub-annular space occurs before expanding the self-expanding tubular body across the native mitral valve and deploying the atrial skirt against the wall of the native mitral annulus and native left atrium.
